(19) United States
(12) Patent Application Publication
(10) Pub. No.: US 2011/0245156 A1
(43) Pub. Date: Oct. 6, 2011

(54) NOVEL ANTIVIRAL COMPOUNDS, COMPOSITIONS, AND METHODS OF USE

(75) Inventor: Thais M. Sielecki-Dzurdz, Kennett Square, PA (US)

(73) Assignee: CYTOKINE PHARMASCIENCES, INC., King of Prussia, PA (US)

(21) Appl. No.: 13/133,492

(22) PCT Filed: Dec. 9, 2009
(86) PCT No.: PCT/US2009/067335

§ 371 Date: Jun. 8, 2011

Related U.S. Application Data
(60) Provisional application No. 61/120,948, filed on Dec. 9, 2008.

(51) Int. Cl. A61K 31/505; C07D 239/48; C07F 9/6512; C07D 403/12; C07D 403/04; C07D 413/12; C07D 413/04; C07D 419/12; C07D 419/04; C07D 471/04; C07D 473/00; C07D 491/052; A61K 31/675; A61K 31/506; A61K 31/496; A61K 31/53; A61K 31/5377; A61K 31/5355; A61K 31/54; A61K 31/55; A61K 31/551; A61K 31/7072; A61K 38/16; A61P 31/14; A61P 31/12; A61K 31/708; A61P 31/18; A61P 31/16; A61P 31/22

(52) U.S. Cl. 514/3.8; 544/323; 544/243; 544/324; 544/238; 544/296; 544/182; 544/122; 544/63; 544/2; 540/601; 540/553; 514/275; 514/86; 514/252.02; 514/252.14; 514/242; 514/235.8; 514/228.8; 514/222.5; 514/217.06; 514/218; 514/50; 514/4.3; 514/3.7; 514/45

(57) ABSTRACT

Compounds are disclosed. Compositions that include the compounds are disclosed. Methods of making and using the compounds are also disclosed.

### CROSS-REFERENCE TO RELATED APPLICATIONS

This application is based on and claims priority to U.S. Provisional Application No. 61/120,948, filed Dec. 9, 2008, the entire contents of which are hereby incorporated by reference.

### BACKGROUND

1. Field

The present invention is directed to compounds, compositions, methods of making, and methods of using. Such compounds may be suitable, for example, in medicine.

2. Related Art

The relevant contents of each of the following documents are incorporated by reference: U.S. Pat. Nos. 5,574,040; 5,840,893; 5,808,068; 5,849,793; 5,877,282; and 6,297,253; U.S. Published Application No. 2005/0203150; Gendelman et al., J. Virol. 58:67-74, 1986; Gartner et al., Science 233:215-219, 1986; Langhoff et al., Proc. Natl. Acad. Sci. USA 88:998-8002, 1991; Ramazzotti et al., Immunology 85:94-98, 1995; Bukrinsky et al., Proc. Natl. Acad. Sci. USA 89:6580-6584, 1992; Bukrinsky et al., —Nature 365:666-669, 1993; von Schwedler et al., Proc. Natl. Acad. Sci. USA 91:6992-6996, 1994; Lewis et al., EMBO J. 11:3053-3058, 1992; Roe et al., EMBO J. 12:2099-2108, 1993; Lewis and Emerman, J. Virol. 68:510-516, 1994; Gallay et al., J. Virol. 70:1027-1032, 1996; Popov et al., Proc. Natl. Acad. Sci. USA 93:11859-11864, 1996; Adam, Trends Cell Biol. 5:189-191, 1995; Radu et al., Proc. Natl. Acad. Sci. USA 92:1769-1773, 1995; Moroianu et al., Proc. Natl. Acad. Sci. USA 92:2008-2011, 1995; Gorlich et al., —Nature (London) 377:246-248, 1995; Adam and Gerace, Cell 66:837-847, 1991; Gorlich and Mattaj, Science 271:1513-1518, 1996; Hurt, Cell 84:509-515, 1996; Bukrinsky et al., —Nature 365:666-669, 1993; von Schwedler et al., Proc. Natl. Acad. Sci. USA 91:6992-6996, 1994; Gallay et al., J. Virol. 70:1027-1032, 1996; Bukrinsky et al., Proc. Natl. Acad. Sci. USA 90:6125-6129, 1993; Haffar et al., J. Mol. Biol. 299: 359-368, 2000; von Schwedler et al., Proc. Natl. Acad. Sci. USA 91:6992-6996, 1994; Heizinger et al., Proc. Natl. Acad. Sci. USA 91:7311-7315, 1992; Bukrinsky et al., —Nature 365:666-669, 1993; Emerman et al., —Nature (London) 369:107-108, 1994; Popov et al., Proc. Natl. Acad. Sci. USA 93:11859-11864, 1996; Nadler et al., J. Biol. Chem. 272, 4310-4315, 1997; Lanford et al., Cell 15: 575-582, 1986; Lanford et al., Mol. Cell. Biol. 8:2722-2729, 1988; Dworetzky et al., J. Cell Biol. 106: 575-584, 1988; Adam et al., Cell 66:837-847, 1991; Hariton-Gazal et al., Bio. Biophysic. Acta 1584: 234-242, 2002; Fletcher et al., EMBO J. 1996 Nov. 15; 15(22):6155-65; Igarashi T, et al., Proc Natl Acad Sci USA. 2001 Jan. 16; 98(2):658-63; Glushakova S, et al., Antiviral Res. 2000 August; 47(2):89-95; and Haffar O. K., et al., Antimicrob Agents Chemother. 1998 May; 42(5):1133-8.

### DESCRIPTION OF THE SEVERAL EMBODIMENTS

One embodiment relates to a compound having the following formula:

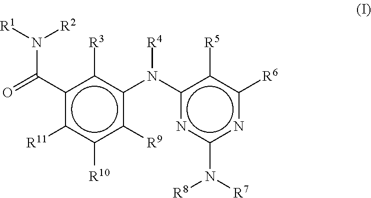

or salt thereof.

The compounds are useful to treat and/or prevent known or suspected maladies in an animal subject. For example, the compounds may be suitably administered to a animal known or suspected to have one or more maladies such as HIV infection, hepatitis C, hepatitis B, hepatitis delta, influenza, herpes, adenovirus, papillomavirus, parvovirus, bird flu, and/or measles, which includes administering one or more of the compounds or compositions herein to the animal. Without wishing to be bound by theory, in one embodiment, these compounds may exercise their beneficial properties via a nuclear importation inhibition mechanism. In another embodiment, these compounds may exercise their beneficial properties through other mechanisms of action, including those that do not involve nuclear importation inhibition.

The animal subject may include, for example, a mammal or avian subject. Exemplary mammals include, for example, a human, dog, cat, pig, horse, cow, mouse, or the like. Exemplary avian subjects include, for example, a duck, chicken, blackbird, goose, crow, pigeon, turkey, squab, swan, and the like.

In one embodiment, the compounds are useful to treat and/or prevent known or suspected maladies in a human subject. For example, the compounds may be suitably administered to a human known or suspected to have one or more maladies such as HIV infection, hepatitis C, hepatitis B, hepatitis delta, influenza, herpes, adenovirus, papillomavirus, parvovirus, bird flu, and/or measles, which includes administering one or more of the compounds or compositions herein to the human. Without wishing to be bound by theory, these compounds may exercise their beneficial properties via a nuclear importation inhibition mechanism.

In one embodiment, the compounds may be suitably administered to a human, dog, cat, pig, horse, cow, mouse, duck, chicken, blackbird, goose, crow, pigeon, turkey, squab, swan, and the like known or suspected to have bird flu for the treatment and/or the prevention of same.

The compounds and compositions herein are useful for treating a subject suspected to have or known to have one or more of the maladies listed herein or elsewhere. As used herein, the terms “treat”, “treating” and/or “treatment” refers to acting upon with the compound or composition to improve or alter an outcome. The skilled artisan is aware that the improvement or alteration may be in whole or in part and may not be a complete cure. Treating may also include treating a subject at risk for developing one or more of the maladies recited herein or elsewhere.

In the formula above, each of R¹ and R² may be independently selected from the group including hydrogen, hydroxyl, halo, bromo, chloro, iodo, fluoro, —NR¹²COR¹³, CONR¹²R¹³, —CONHR¹², —COOR¹², —OCOR¹², —COR¹², —SR¹², —SO₂R¹², —SO₃R¹², —SO₂NHR¹², —SOR¹², —CN, —NC, —SH, —NO₂, —NH₂, —PR₂, —(O)PR¹²R¹³, PO₃R¹²R¹³, —OPO₃R¹²R¹³, —PO₂, (C₁-C₂₀) alkyl, phenyl, (C₃-C₂₀) cycloalkyl, (C₁-C₂₀) alkoxy, (C₃-C₂₅) heteroaryl, (C₃-C₂₅) heterocyclyl, (C₂-C₂₀) alkenyl, (C₄-C₂₀) cycloalkenyl, (C₂-C₂₀) alkynyl, (C₆-C₂₀) cycloalkynyl, (C₅-C₂₅) aryl, perhalo (C₁-C₂₀) alkyl, and salt thereof.

R¹ and R² together with the nitrogen to which they are attached may form a heterocyclic ring group or salt thereof.

Each of R³, R⁹, and R¹¹ may be independently selected from the group including hydrogen, hydroxyl, halo, bromo, chloro, iodo, fluoro, halo, bromo, chloro, iodo, fluoro, OR¹², —NR¹²R¹³, —NR¹²COR¹³, CONR¹²R¹³, —CONHR¹², —COOR¹², —OCOR¹², COR¹², —SR¹², —SO₂R¹², —SO₃R¹², —SO₂NHR¹², —SOR¹², —N₃, —CN, —NC, —SH, NO₂, —NH₂, —PR₂, —(O)PR¹²R¹³, —PO₃R¹²R¹³, —OPO₃R¹²R¹³, —PO₂, (C₁-C₂₀) alkyl, phenyl, (C₃-C₂₀) cycloalkyl, (C₁-C₂₀) alkoxy, (C₃-C₂₅) heteroaryl, (C₃-C₂₅) heterocyclyl, (C₂-C₂₀) alkenyl, (C₄-C₂₀) cycloalkenyl, (C₂-C₂₀) alkynyl, (C₆-C₂₀) cycloalkynyl, (C₅-C₂₅) aryl, perhalo (C₁-C₂₀) alkyl, and salt thereof.

R⁴ may be hydrogen, hydroxyl, halo, bromo, chloro, iodo, fluoro, —OR¹², —NR¹²R¹³, NR¹²COR¹³, —CONR¹²R¹³, CONHR¹², —COOR¹², —OCOR¹², —COR¹², —SR¹², SO₂R¹², —SO₃R¹², —SO₂NHR¹², —SOR¹², —CN, —NC, —SH, —NO₂, —NH₂, —PR₂, (O)PR¹²R¹³, —PO₃R¹²R¹³, OPO₃R¹²R¹³, —PO₂, (C₁-C₂₀) alkyl, phenyl, (C₃-C₂₀) cycloalkyl, (C₁-C₂₀) alkoxy, (C₃-C₂₅) heteroaryl, (C₃-C₂₅) heterocyclyl, (C₂-C₂₀) alkenyl, (C₄-C₂₀) cycloalkenyl, (C₂-C₂₀) alkynyl, (C₆-C₂₀) cycloalkynyl, (C₅-C₂₅) aryl, perhalo (C₁-C₂₀) alkyl, or salt thereof.

Each of R⁵ and R⁶ may be independently selected from the group including hydrogen, hydroxyl, halo, bromo, chloro, iodo, fluoro, —OR¹², —NR¹²R¹³, —NR¹²COR¹³, CONR¹²R¹³, —CONHR¹², —COOR¹², —OCOR¹², —COR¹², —SR¹², —SO₂R¹², —SO₃R¹², —SO₂NHR¹², —SOR¹², —N₃, —CN, —NC, —SH, —NO₂, —NH₂, —PR₂, —(O)PR¹²R¹³, —PO₃R¹²R¹³, —OPO₃R¹²R¹³, —PO₂, (C₁-C₂₀) alkyl, phenyl, (C₃-C₂₀) cycloalkyl, (C₁-C₂₀) alkoxy, (C₃-C₂₅) heteroaryl, (C₃-C₂₅) heterocyclyl, (C₂-C₂₀) alkenyl, (C₄-C₂₀) cycloalkenyl, (C₂-C₂₀) alkynyl, (C₆-C₂₀) cycloalkynyl, (C₅-C₂₅) aryl, perhalo (C₁-C₂₀) alkyl, and salt thereof.

R⁵ and R⁶ together with the carbons to which they are attached may also form a (C₅-C₆) aryl structure, a (C₃-C₆) heteroaryl structure, a (C₃-C₆) heterocycloalkenyl structure, a (C₄-C₆) cycloalkenyl structure, or salt thereof.

Each of R⁷ and R⁸ may be independently selected from the group including hydrogen, hydroxyl, halo, bromo, chloro, iodo, fluoro, —C(NH)NH₂, —C(NH)NHC(NH)NH₂, —OR¹², —NR¹²R¹³, —NR¹²COR¹³, —CONR¹²R¹³, —CONHR¹², —COOR¹², —OCOR¹², —COR¹², —SR¹², —SO₂R¹², —SO₃R¹², —SO₂NHR¹², —SOR¹², —CN, —NC, —SH, —NO₂, —NH₂, —PR₂, —(O)PR¹²R¹³, —PO₃R¹²R¹³, —OPO₃R¹²R¹³, —PO₂, (C₁-C₂₀) alkyl, phenyl, (C₃-C₂₀) cycloalkyl, (C₁-C₂₀) alkoxy, (C₃-C₂₅) heteroaryl, (C₃-C₂₅) heterocyclyl, (C₂-C₂₀) alkenyl, (C₄-C₂₀) cycloalkenyl, (C₂-C₂₀) alkynyl, (C₆-C₂₀) cycloalkynyl, (C₅-C₂₅) aryl, perhalo (C₁-C₂₀) alkyl, and salt thereof.

R⁷ and R⁸ together with the nitrogen to which they are attached may form a heterocyclic ring group or salt thereof.

R¹⁰ may be hydrogen, hydroxyl, halo, bromo, chloro, iodo, fluoro, —OR¹⁴, NR¹⁴R¹⁵, —NR¹⁴COR¹⁵, —CONR¹⁴R¹⁵, —CONR¹⁴, —COOR¹⁴, —OCOR¹⁴, —COR¹⁴, SR¹⁴, —SO₂R¹⁴, —SO₃R¹⁴, —SO₂NR¹⁴, —SOR¹⁴, —N₃, —CN, —NC, —SH, —NO₂, NH₂, —PR₂, —(O)PR¹⁴R¹⁵, —PO₃R¹⁴R¹⁵, —OPO₃R¹⁴R¹⁵, —PO₂, (C₁-C₂₀) alkyl, phenyl, (C₃-C₂₀) cycloalkyl, (C₁-C₂₀) alkoxy, (C₃-C₂₅) heteroaryl, (C₃-C₂₅) heterocyclyl, (C₂-C₂₀) alkenyl, (C₄-C₂₀) cycloalkenyl, (C₂-C₂₀) alkynyl, (C₆-C₂₀) cycloalkynyl, (C₅-C₂₅) aryl, perhalo (C₁-C₂₀) alkyl, or salt thereof.

Each of R¹² and R¹³ may be each independently selected from the group including (C₁-C₂₀) alkyl, phenyl, (C₃-C₂₀) cycloalkyl, (C₁-C₂₀) alkoxy, (C₃-C₂₅) heteroaryl, (C₃-C₂₅) heterocyclyl, (C₂-C₂₀) alkenyl, (C₄-C₂₀) cycloalkenyl, (C₂-C₂₀) alkynyl, (C₆-C₂₀) cycloalkynyl, (C₅-C₂₅) aryl, perhalo (C₁-C₂₀) alkyl, and salt thereof.

Each of R¹⁴ and R¹⁵ may be independently selected from the group including hydrogen, hydroxyl, halo, bromo, chloro, iodo, fluoro, —OR¹², —NR¹²R¹³, —NR¹²COR¹³, CONR¹²R¹³, —CONHR¹², —COOR¹², —OCOR¹², —COR¹², —SR¹², —SO₂R¹², —SO₃R¹², —SO₂NHR¹², —SOR¹², —N₃, —CN, —NC, —SH, —NO₂, —NH₂, —PR₂, —(O)PR¹²R¹³, —PO₃R¹²R¹³, —OPO₃R¹²R¹³, —PO₂, (C₁-C₂₀) alkyl, phenyl, (C₃-C₂₀) cycloalkyl, (C₁-C₂₀) alkoxy, (C₃-C₂₅) heteroaryl, (C₃-C₂₅) heterocyclyl, (C₂-C₂₀) alkenyl, (C₄-C₂₀) cycloalkenyl, (C₂-C₂₀) alkynyl, (C₆-C₂₀) cycloalkynyl, (C₅-C₂₅) aryl, perhalo (C₁-C₂₀) alkyl, and salt thereof.

Each of the (C₁-C₂₀) alkyl, phenyl, (C₃-C₂₀) cycloalkyl, (C₁-C₂₀) alkoxy, (C₃-C₂₅) heteroaryl, (C₃-C₂₅) heterocyclyl, (C₂-C₂₀) alkenyl, (C₄-C₂₀) cycloalkenyl, (C₂-C₂₀) alkynyl, (C₆-C₂₀) cycloalkynyl, (C₅-C₂₅) aryl, perhalo (C₁-C₂₀) alkyl, (C₃-C₆) cycloalkyl structure, (C₅-C₆) aryl structure, (C₃-C₆) heteroaryl structure, (C₃-C₆) heterocyclyl structure, (C₃-C₆) heterocycloalkenyl structure, (C₄-C₆) cycloalkenyl structure, heterocyclic ring group, or salt thereof may be independently unsubstituted or substituted. If substituted, there may be one or more than one substituent. Combinations of substituents are possible. Exemplary substituents include hydroxyl, halo, bromo, chloro, iodo, fluoro, —OR¹², —NR¹²R¹³, NR¹²COR¹³, —CONR¹²R¹³, CONHR¹², —COOR¹², —OCOR¹², —COR¹², —SR¹², SO₂R¹², —SO₃R¹², —SO₂NHR¹², —SOR¹², —N₃, —CN, —NC, —SH, —NO₂, —NH₂, PR₂, —(O)PR¹²R¹³, —OPO₃R¹²R¹³, OPO₃R¹²R¹³, —PO₂, (C₁-C₂₀) alkyl, phenyl, (C₃-C₂₀) cycloalkyl, (C₁-C₂₀) alkoxy, (C₃-C₂₅) heteroaryl, (C₃-C₂₅) heterocyclyl, (C₂-C₂₀) alkenyl, (C₄-C₂₀) cycloalkenyl, (C₂-C₂₀) alkynyl, (C₆-C₂₀) cycloalkynyl, (C₅-C₂₅) aryl, perhalo (C₁-C₂₀) alkyl, salt thereof, and a combination thereof.

In one embodiment, in the formula (I) above, when R³, R¹⁰, and R¹¹ are hydrogen, and R⁹ is —CH₃, then R⁵ is not —CN.

In one embodiment, each of R¹ and R² may be independently selected from the group including hydrogen, hydroxyl, halo, bromo, chloro, iodo, and fluoro.

In one embodiment, each of R¹ and R² may be independently selected from the group including —NR¹²COR¹³, —CONR¹²R¹³, —CONHR¹², —COOR¹², —OCOR¹², —COR¹², SR¹², —SO₂R¹², —SO₃R¹², —SO₂NHR¹², —SOR¹², —CN, —NC, —SH, —NO₂, —NH₂, —PR₂, —(O)PR¹²R¹³, —OPO₃R¹²R¹³, OPO₃R¹²R¹³, —PO₂, and salt thereof.

In one embodiment, each of R¹ and R² may be independently selected from the group including (C₁-C₂₀) alkyl, phenyl, (C₃-C₂₀) cycloalkyl, (C₁-C₂₀) alkoxy, (C₃-C₂₅) heteroaryl, (C₃-C₂₅) heterocyclyl, (C₂-C₂₀) alkenyl, (C₄-C₂₀) cycloalkenyl, (C₂-C₂₀) alkynyl, (C₆-C₂₀) cycloalkynyl, (C₅-C₂₅) aryl, perhalo (C₁-C₂₀) alkyl, and salt thereof.

In one embodiment, R¹ and R² together with the nitrogen to which they are attached form a heterocyclic ring group or salt thereof.

In one embodiment, each of R³, R⁹, and R¹¹ may be independently selected from the group including hydrogen, hydroxyl, halo, bromo, chloro, iodo, fluoro.

In one embodiment, each of R³, R⁹, and R¹¹ may be independently selected from the group including —OR¹², —NR¹²R¹³, —NR¹²COR¹³, —CONR¹²R¹³, CONHR¹², COOR¹², —OCOR¹², —COR¹², —SR¹², —SO₂R¹², —SO₃R¹², —SO₂NHR¹², —SOR¹², N₃, —CN, —NC, —SH, —NO₂, —NH₂, —PR₂, —(O)PR¹²R¹³, —PO₃R¹²R¹³, OPO₃R¹²R¹³, —PO₂, and salt thereof.

In one embodiment, each of R³, R⁹, and R¹¹ may be independently selected from the group including (C₁-C₂₀) alkyl, phenyl, (C₃-C₂₀) cycloalkyl, (C₁-C₂₀) alkoxy, (C₃-C₂₅) heteroaryl, (C₃-C₂₅) heterocyclyl, (C₂-C₂₀) alkenyl, (C₄-C₂₀) cycloalkenyl, (C₂-C₂₀) alkynyl, (C₆-C₂₀) cycloalkynyl, (C₅-C₂₅) aryl, perhalo (C₁-C₂₀) alkyl, and salt thereof.

In one embodiment, R⁴ may be selected from the group including hydrogen, hydroxyl, halo, bromo, chloro, iodo, and fluoro.

In one embodiment, R⁴ may be selected from the group including —OR¹², —NR¹²R¹³, —NR¹²COR¹³, —CONR¹²R¹³, —CONHR¹², —COOR¹², —OCOR¹², —COR¹², —SR¹², SO₂R¹², —SO₃R¹², —SO₂NHR¹², —SOR¹², —CN, —NC, —SH, —NO₂, —NH₂, —PR₂, (O)PR¹²R¹³, —PO₃R¹²R¹³, —OPO₃R¹²R¹³, —PO₂, and salt thereof.

In one embodiment, R⁴ may be selected from the group including (C₁-C₂₀) alkyl, phenyl, (C₃-C₂₀) cycloalkyl, (C₁-C₂₀) alkoxy, (C₃-C₂₅) heteroaryl, (C₃-C₂₅) heterocyclyl, (C₂-C₂₀) alkenyl, (C₄-C₂₀) cycloalkenyl, (C₂-C₂₀) alkynyl, (C₆-C₂₀) cycloalkynyl, (C₅-C₂₅) aryl, perhalo (C₁-C₂₀) alkyl, and salt thereof.

In one embodiment, each of R⁵ and R⁶ may be independently selected from the group including hydrogen, hydroxyl, halo, bromo, chloro, iodo, and fluoro.

In one embodiment, each of R⁵ and R⁶ may be independently selected from the group including —OR¹², —NR¹²R¹³, —NR¹²COR¹³, —CONR¹²R¹³, —CONHR¹², —COOR¹², OCOR¹², —COR¹², —SR¹², —SO₂R¹², —SO₃R¹², —SO₂NHR¹², —SOR¹², —N₃, —CN, NC, —SH, —NO₂, —NH₂, —PR₂, —(O)PR¹²R¹³, —OPO₃R¹²R¹³, OPO₃R¹²R¹³, —PO₂, and salt thereof.

In one embodiment, each of R⁵ and R⁶ may be independently selected from the group including (C₁-C₂₀) alkyl, phenyl, (C₃-C₂₀) cycloalkyl, (C₁-C₂₀) alkoxy, (C₃-C₂₅) heteroaryl, (C₃-C₂₅) heterocyclyl, (C₂-C₂₀) alkenyl, (C₄-C₂₀) cycloalkenyl, (C₂-C₂₀) alkenyl, (C₆-C₂₀) cycloalkynyl, (C₅-C₂₅) aryl, perhalo (C₁-C₂₀) alkyl, and salt thereof.

In one embodiment, R⁵ and R⁶ together with the carbons to which they are attached form a (C₅-C₆) aryl structure, a (C₃-C₆) heteroaryl structure, a (C₃-C₆) heterocycloalkenyl structure, a (C₄-C₆) cycloalkenyl structure, or salt thereof.

In one embodiment, each of R⁷ and R⁸ may be independently selected from the group including hydrogen, hydroxyl, halo, bromo, chloro, iodo, and fluoro.

In one embodiment, each of R⁷ and R⁸ may be independently selected from the group including —C(NH)NH₂, —C(NH)NHC(NH)NH₂, —OR¹², —NR¹²R¹³, —NR¹²COR¹³, CONR¹²R¹³, —CONHR¹², —COOR¹², —OCOR¹², —COR¹², —SR¹², —SO₂R¹², —SO₃R¹², —SO₂NHR¹², —SOR¹², —CN, —NC, —SH, —NO₂, —NH₂, —PR₂, —(O)PR¹²R¹³, PO₃R¹²R¹³, —OPO₃R¹²R¹³, —PO₂, and salt thereof.

In one embodiment, each of R⁷ and R⁸ may be independently selected from the group including (C₁-C₂₀) alkyl, phenyl, (C₃-C₂₀) cycloalkyl, (C₁-C₂₀) alkoxy, (C₃-C₂₅) heteroaryl, (C₃-C₂₅) heterocyclyl, (C₂-C₂₀) alkenyl, (C₄-C₂₀) cycloalkenyl, (C₂-C₂₀) alkenyl, (C₆-C₂₀) cycloalkynyl, (C₅-C₂₅) aryl, perhalo (C₁-C₂₀) alkyl, and salt thereof.

In one embodiment, R⁷ and R⁸ together with the nitrogen to which they are attached form a heterocyclic ring group or salt thereof.

In one embodiment, R¹⁰ may be selected from the group including hydrogen, hydroxyl, halo, bromo, chloro, iodo, and fluoro.

In one embodiment, R¹⁰ may be selected from the group including —OR¹⁴, —NR¹⁴R¹⁵, —NR¹⁴COR¹⁵, —CONR¹⁴R¹⁵, —CONR¹⁴, —COOR¹⁴, —OCOR¹⁴, —COR¹⁴, SR¹⁴, —SO₂R¹⁴, —SO₃R¹⁴, —SO₂NR¹⁴, —SOR¹⁴, —N₃, —CN, —NC, —SH, —NO₂, NH₂, —PR₂, —(O)PR¹⁴R¹⁵, —PO₃R¹⁴R¹⁵, OPO₃R¹⁴R¹⁵, —PO₂, and salt thereof.

In one embodiment, R¹⁰ may be selected from the group including (C₁-C₂₀) alkyl, phenyl, (C₃-C₂₀) cycloalkyl, (C₁-C₂₀) alkoxy, (C₃-C₂₅) heteroaryl, (C₃-C₂₅) heterocyclyl, (C₂-C₂₀) alkenyl, (C₄-C₂₀) cycloalkenyl, (C₂-C₂₀) alkynyl, (C₆-C₂₀) cycloalkynyl, (C₅-C₂₅) aryl, perhalo (C₁-C₂₀) alkyl, and salt thereof.

In one embodiment, each of R¹⁴ and R¹⁵ may be independently selected from the group including hydrogen, hydroxyl, halo, bromo, chloro, iodo, and fluoro.

In one embodiment, each of R¹⁴ and R¹⁵ may be independently selected from the group including —OR¹², —NR¹²R¹³, —NR¹²COR¹³, —CONR¹²R¹³, CONHR¹², COOR¹², —OCOR¹², —COR¹², —SR¹², —SO₂R¹², —SO₃R¹², —SO₂NHR¹², —SOR¹², N₃, —CN, —NC, —SH, —NO₂, —NH₂, —PR₂, —(O)PR¹²R¹³, —PO₃R¹²R¹³, OPO₃R¹²R¹³, —PO₂, and salt thereof.

In one embodiment, each of R¹⁴ and R¹⁵ may be independently selected from the group including (C₁-C₂₀) alkyl, phenyl, (C₃-C₂₀) cycloalkyl, (C₁-C₂₀) alkoxy, (C₃-C₂₅) heteroaryl, (C₃-C₂₅) heterocyclyl, (C₂-C₂₀) alkenyl, (C₄-C₂₀) cycloalkenyl, (C₂-C₂₀) alkynyl, (C₆-C₂₀) cycloalkynyl, (C₅-C₂₅) aryl, perhalo (C₁-C₂₀) alkyl, and salt thereof.

In one embodiment, at least one (C₁-C₂₀) alkyl, phenyl, (C₃-C₂₀) cycloalkyl, (C₁-C₂₀) alkoxy, (C₃-C₂₅) heteroaryl, (C₃-C₂₅) heterocyclyl, (C₂-C₂₀) alkenyl, (C₄-C₂₀) cycloalkenyl, (C₂-C₂₀) alkynyl, (C₆-C₂₀) cycloalkynyl, (C₅-C₂₅) aryl, perhalo (C₁-C₂₀) alkyl, (C₃-C₆) cycloalkyl structure, (C₅-C₆) aryl structure, (C₃-C₆) heteroaryl structure, (C₃-C₆) heterocyclyl structure, (C₃-C₆) heterocycloalkenyl structure, (C₄-C₆) cycloalkenyl structure, heterocyclic ring group, or salt thereof is substituted with one or more substituents selected from the group consisting of hydroxyl, halo, bromo, chloro, iodo, fluoro, —OR¹², —NR¹²R¹³, —NR¹²COR¹³, —CONR¹²R¹³, —CONHR¹², —COOR¹², —OCOR¹², —COR¹², —SR¹², —SO₂R¹², —SO₃R¹², —SO₂NHR¹², —SOR¹², —N₃, —CN, —NC, —SH, —NO₂, —NH₂, —PR₂, (O)PR¹²R¹³, —PO₃R¹²R¹³, —OPO₃R¹²R¹³, —PO₂, (C₁-C₂₀) alkyl, phenyl, (C₃-C₂₀) cycloalkyl, (C₁-C₂₀) alkoxy, (C₃-C₂₅) heteroaryl, (C₃-C₂₅) heterocyclyl, (C₂-C₂₀) alkenyl, (C₄-C₂₀) cycloalkenyl, (C₂-C₂₀) alkynyl, (C₆-C₂₀) cycloalkynyl, (C₅-C₂₅) aryl, perhalo (C₁-C₂₀) alkyl, salt thereof, and a combination thereof.

In one embodiment, at least one (C₁-C₂₀) alkyl, phenyl, (C₃-C₂₀) cycloalkyl, (C₁-C₂₀) alkoxy, (C₃-C₂₅) heteroaryl, (C₃-C₂₅) heterocyclyl, (C₂-C₂₀) alkenyl, (C₄-C₂₀) cycloalkenyl, (C₂-C₂₀) alkynyl, (C₆-C₂₀) cycloalkynyl, (C₅-C₂₅) aryl, perhalo (C₁-C₂₀) alkyl, (C₃-C₆) cycloalkyl structure, (C₅-C₆) aryl structure, (C₃-C₆) heteroaryl structure, (C₃-C₆) heterocyclyl structure, (C₃-C₆) heterocycloalkenyl structure, (C₄-C₆) cycloalkenyl structure, heterocyclic ring group, or salt thereof is not substituted.

In one embodiment, at least one of R³, R¹⁰, or R¹¹ is not hydrogen.

In one embodiment, R⁹ is not —CH₃.

In one embodiment, the R¹ and R² together with the nitrogen to which they are attached, or the R⁷ and R⁸ together with the nitrogen to which they are attached, or both R¹ and R² and R⁷ and R⁸, respectively together with the nitrogens to which they are attached, independently forms a heterocyclic ring group or salt thereof. Exemplary hetrocyclic ring groups include:

pyrrolyl, 2,3-dihydropyrrol-1-yl, 2,5-dihydropyrrol-1-yl, tetrahydropyrrol-1-yl,

pyrazol-1-yl, 3H-pyrazol-1-yl, 4H-pyrazol-1-yl, 3,4-dihydropyrazol-1-yl,

1,3-isodiazol-1-yl, 3H-(1,3-isodiazol-1-yl), 3,4-dihydro-(1,3-isodiazol-1-yl),

1,2,3-triazol-1-yl, 2H-(1,2,3-triazol-1-yl), 2,3-dihydro(1,2,3-triazol-1-yl), 2,3,4-trihydro(1,2,3-triazol-1-yl),

1,2,3-triazol-2-yl, 1,3-dihydro(1,2,3-triazol-2-yl), 1,3,4-trihydro(1,2,3-triazol-2-yl),

1,2,4-triazol-1-yl, 2H-(1,2,4-triazol-1-yl), 2,3-dihydro(1,2,4-triazol-1-yl), 2,4-dihydro(1,2,4-triazol-1-yl),

1,2,4-triazol-4-yl, 1H-(1,2,4-triazol-4-yl), 1,2-dihydro(1,2,4-triazol-4-yl), 3,5-dihydro(1,2,4-triazol-4-yl),

isoxazol-2-yl, 3H-isoxazol-2-yl, 4H-isoxazol-2-yl,

oxazol-3-yl, 4H-oxazol-3-yl,

thiazol-3-yl, 4H-thiazol-3-yl,

isothiazol-2-yl, 3H-isothiazol-2-yl,

1,2,3-oxadiazol-2-yl, 3H-(1,2,3-oxadiazol-2-yl),

1,2,3-oxadiazol-3-yl, 4H-(1,2,3-oxadiazol-3-yl),

1,2,4-oxidiazol-2-yl, 3H-(1,2,4-oxidiazol-2-yl), 4H-(1,2,4-oxidiazol-2-yl), 5H-(1,2,4-oxidiazol-2-yl),

1,2,4-oxidiazol-4-yl, 3H-(1,2,4-oxidiazol-4-yl),

1,2,5-oxadiazol-2-yl, 4H-(1,2,5-oxadiazol-2-yl),

1,3,4-oxadiazol-3-yl, 4H-(1,3,4-oxadiazol-3-yl),

1,2,3,4-oxatriazol-2-yl, 3H-(1,2,3,4-oxatriazol-2-yl), 5H-(1,2,3,4-oxatriazol-2-yl), 3H-, 4H-(1,2,3,4-oxatriazol-2-yl),

1,2,3,4-oxatriazol-3-yl, 4H-(1,2,3,4-oxatriazol-3-yl),

5H-(1,2,3,5-oxatriazol-2-yl), 3H-,4H-(1,2,3,5-oxatriazol-2-yl),

1,2,3,5-oxatriazol-3-yl, 4H-(1,2,3,5-oxatriazol-3-yl),

1,2,3,5-oxatriazol-5-yl, 4H-(1,2,3,5-oxatriazol-5-yl), 2H-(1,2,3,5-oxatriazol-5-yl), 2H-,3H-(1,2,3,5-oxatriazol-5-yl),

1,2,3-dioxazol-3-yl, 4H-(1,2,3-dioxazol-3-yl),

1,2,4-dioxazol-4-yl,

1,3,2-dioxazol-2-yl, 4H-(1,3,2-dioxazol-2-yl),

1,3,4-dioxazol-4-yl,

1,2,5-oxathiazol-5-yl, 3H-(1,2,5-oxathiazol-5-yl),

2H-(pyridin-1-yl), 4H-(pyridin-1-yl), 3H-,4H-(pyridin-1-yl), 2H-,5H-(pyridin-1-yl), 3H-,5H-(pyridin-1-yl),

2H-(pyridazin-1-yl), 4H-(pyridazin-1-yl), 6H-(pyridazin-1-yl), 3H-,4H-(pyridazin-1-yl), 4H-,5H-(pyridazin-1-yl), 2H-,5H-(pyridazin-1-yl), 2H-,3H-,6H-(pyridazin-1-yl), 3H-,5H-(pyridazin-1-yl),

2H-(pyrimidin-1-yl), 4H-(pyrimidin-1-yl), 6H-(pyrimidin-1-yl), 3H-,4H-(pyrimidin-1-yl), 4H-,5H-(pyrimidin-1-yl), 2H-,5H-(pyrimidin-1-yl), 2H-,3H-,6H-(pyrimidin-1-yl), 3H-, 5H-(pyrimidin-1-yl),

2H-(pyrazin-1-yl), 4H-(pyrazin-1-yl), 3H-,4H-(pyrazin-1-yl), 2H-,5H-(pyrazin-1-yl), piperazin-1-yl,

2H-(s-triazin-1-yl), 4H-(s-triazin-1-yl), 4H-,5H-(s-triazin-1-yl), 2H-,5H-(s-triazin-1-yl), 3H-,5H-(s-triazin-1-yl),

2H-(as-triazin-1-yl), 4H-(as-triazin-1-yl), 6H-(as-triazin-1-yl), 3H-,4H-(as-triazin-1-yl), 4H-,5H-(as-triazin-1-yl), 2H-,5H-(as-triazin-1-yl), 2H-,3H-,6H-(as-triazin-1-yl), 3H-,5H-(as-triazin-1-yl),

1H-(as-triazin-2-yl), 3H-(as-triazin-2-yl), 5H-,6H-(as-triazin-2-yl), 3H-,6H-(as-triazin-2-yl), 1H-,4H-(as-triazin-2-yl), 5H-(as-triazin-2-yl), 1H-,3H-,5H-(as-triazin-2-yl), 3H-,5H-(as-triazin-2-yl),

1H-(as-triazin-4-yl), 3H-(as-triazin-4-yl), 5H-(as-triazin-4-yl), 3H-,6H-(as-triazin-4-yl), 2H-,5H-(as-triazin-4-yl), 1H-,5H-(as-triazin-4-yl), 1H-,3H-(as-triazin-4-yl), 1H-,3H-,5H-(as-triazin-4-yl),

2H-(v-triazin-1-yl), 4H-(v-triazin-1-yl), 6H-(v-triazin-1-yl), 3H-,4H-(v-triazin-1-yl), 4H-,5H-(v-triazin-1-yl), 2H-,5H-(v-triazin-1-yl), 2H-,3H-,6H-(v-triazin-1-yl), 3H-,5H-(v-triazin-1-yl),

1H-(v-triazin-2-yl), 3H-(v-triazin-2-yl), 3H-,5H-(v-triazin-2-yl), 3H-,6H-(v-triazin-2-yl), 5H-,6H-(v-triazin-2-yl), 5H-(v-triazin-2-yl), 1H-,3H-,5H-(v-triazin-2-yl),

1,2-oxazin-2-yl, 5H-,6H-(1,2-oxazin-2-yl), 3H-,6H-(1,2-oxazin-2-yl), 4H-(1,2-oxazin-2-yl), 3H-,5H-(1,2-oxazin-2-yl),

1,2-oxazin-3-yl, 1H-,4H-(1,2-oxazin-2-yl), 2H-,5H-(1,2-oxazin-2-yl),

1,4-oxazin-4-yl, 5H-(1,4-oxazin-4-yl), morpholin-4-yl,

1,2,4-oxathiazin-4-yl, 5H-(1,2,5-oxathiazin-4-yl),

1,2,5-oxathiazin-5-yl, 3H-(1,2,5-oxathiazin-5-yl),

1,2,6-oxathiazin-6-yl, 3H-(1,2,6-oxathiazin-6-yl), 4H-(1,2,6-oxathiazin-6-yl), 5H-(1,2,6-oxathiazin-6-yl),

1,2,4-oxadiazin-4-yl, 2H-(1,2,4-oxadiazin-4-yl), 5H-(1,2,4-oxadiazin-4-yl), 2H-,5H-(1,2,4-oxadiazin-4-yl),

1,3,5-oxadiazin-3-yl, 4H-(1,3,5-oxadiazin-3-yl),

azepin-1-yl, 2H-,7H-azepin-1-yl, 4H-,6H-azepin-1-yl, 3H-,6H-azepin-1-yl, 4H-,7H-azepin-1-yl, 2H-,4H-,6H-azepin-1-yl,

1,2-diazepin-1-yl, 2H-,7H-(1,2-diazepin-1-yl), 4H-,6H-(1,2-diazepin-1-yl), 3H-,6H-(1,2-diazepin-1-yl), 4H-,7H-(1,2-diazepin-1-yl), 2H-,4H-,6H-(1,2-diazepin-1-yl), 2H-,4H-, 7H-(1,2-diazepin-1-yl), 2H-,4H-,5H-(1,2-diazepin-1-yl), 2H-,5H-(1,2-diazepin-1-yl), 2H-, 3H-(1,2-diazepin-1-yl),

indol-1-yl, 6H-,7H-indol-1-yl, 4H-,7H-indol-1-yl, 4H-,5H-indol-1-yl, 7H-,7aH-indol-1-yl, 5H-,7aH-indol-1-yl, 5H-,6H-indol-1-yl, 4aH-,7aH-indol-1-yl, 3aH-,6H-indol-1-yl, 3aH-, 4H-indol-1-yl, 4H-,5H-,6H-,7H-indol-1-yl, 5H-,6H-,7H-,7 aH-indol-1-yl, 6H-,7H-,3aH-,7aH-indol-1-yl, 4H-,7H-,3aH-,7aH-indol-1-yl, 4H-,5H-,3aH-,7aH-indol-1-yl, 4H-,5H-,6H-,3aH-indol-1-yl,

2H-indol-1-yl, 2H-,4aH-,7aH-indol-1-yl, 2H-,3aH-,6H-indol-1-yl, 2H-,3 aH-,4H-indol-1-yl, 2H-,6H-,7H-,3aH-,7aH-indol-1-yl, 2H-,4H-,7H-,3aH-,7aH-indol-1-yl, 2H-,4H-,5H-, 3aH-,7aH-indol-1-yl, 2H-,4H-,5 H-,6H-,3aH-indol-1-yl,

2H-,3H-indol-1-yl, 2H-,3H-,6H-,7H-indol-1-yl, 2H-,3H-,4H-,7H-indol-1-yl, 2H-,3H-, 4H-,5H-indol-1-yl, 2H-,3H-,7H-,7aH-indol-1-yl, 2H-,3H-,5H-,7 aH-indol-1-yl, 2H-,3H-,5H-, 6H-indol-1-yl, 2H-,3H-,4aH-,7 aH-indol-1-yl, 2H-,3H-,3aH-,6H-indol-1-yl, 2H-,3H-,3aH-, 4H-indol-1-yl, 2H-,3 H-,4H-,5 H-,6H-,7H-indol-1-yl, 2H-,3 H-,5 H-,6H-,7 H-,7aH-indol-1-yl, 2H-,3H-,6H-,7H-,3aH-,7aH-indol-1-yl, 2H-,3H-,4H-,7H-,3aH-,7aH-indol-1-yl, 2H-,3H-,4H-, 5H-,3aH-,7aH-indol-1-yl, 2H-,3 H-,4H-,5 H-,6H-,3aH-indol-1-yl,

2-isobenzazol-2-yl, 4H-,5H-(2-isobenzazol-2-yl), 4H-,7H-(2-isobenzazol-2-yl), 1H-, 3H-(2-isobenzazol-2-yl), 1H-,3H-,4H-,5H-(2-isobenzazol-2-yl), 1H-,3H-,4H-,7H-(2-isobenzazol-2-yl), 1H-,3H-,4H-,4aH-,7aH-,7H-(2-isobenzazol-2-yl), 1H-,3H-,4aH-,7aH-(2-isobenzazol-2-yl), 1H-,3H-,5H-,6H-(2-isobenzazol-2-yl), 3H-,4aH-(2-isobenzazol-2-yl), 3H-, 5H-(2-isobenzazol-2-yl), 3H-,7H-(2-isobenzazol-2-yl), 3H-,4aH-,4H-,7aH-,7H-(2-isobenzazol-2-yl), 3H-,4aH-,6H-,7H-(2-isobenzazol-2-yl), 3H-,5H-,6H-,7H-(2-isobenzazol-2-yl), 4H-,4aH-,5H-(2-isobenzazol-2-yl), 1H-,3H-,4aH-,6H-,7H-,7aH-(2-isobenzazol-2-yl), 1H-, 3H-,5H-,6H-,7H-,7aH-(2-isobenzazol-2-yl), 3H-,4H-,4aH-,5H-,6H-,7H-(2-isobenzazol-2-yl), 1H-,3H-,4H-,5H-,6H-,7H-(2-isobenzazol-2-yl), 4H-,5H-,6H-,7H-(2-isobenzazol-2-yl),

1-pyrindin-1-yl, 4H-,5 aH-(1-pyrindin-1-yl), 2H-,5 aH-(1-pyrindin-1-yl), 2H-,3H-,4H-, 5 aH-(1-pyrindin-1-yl), 2H-,7H-,7 aH-(1-pyrindin-1-yl), 4H-,6H-,7H-,7 aH-(1-pyrindin-1-yl), 2H-,3H-,4H-,6H-,7H-,7 aH-(1-pyrindin-1-yl), 2H-,7H-(1-pyrindin-1-yl), 4H-,7H-(1-pyrindin-1-yl), 2H-,3H-,4H-,7H-(1-pyrindin-1-yl), 4H-,5H-,5aH-,7H-,7aH-(1-pyrindin-1-yl), 2H-,3H-, 4H-,5 aH-,7H-,7aH-(1-pyrindin-1-yl), 2H-,3H-,4H-,5H-,6H-,7H-,7aH-(1-pyrindin-1-yl), 2H-, 3H-,4H-,5H-,5 aH-,6H-,7H-,7aH-(1-pyrindin-1-yl), 2H-,5H-(1-pyrindin-1-yl), 4H-,5H-(1-pyrindin-1-yl), 2H-,3H-,4H-,5H-(1-pyrindin-1-yl), 2H-,5H-,5 aH-,7 aH-(1-pyrindin-1-yl), 2H-, 3H-,4H-,5H-,5aH-,7aH-(1-pyrindin-1-yl), 5H-,6H-(1-pyrindin-1-yl), 4H-,5H-,5aH-,6H-(1-pyrindin-1-yl), 2H-,3H-,5H-,6H-(1-pyrindin-1-yl), 2H-,3H-,4H-,5H-,5aH-(1-pyrindin-1-yl), 2H-,6H-(1-pyrindin-1-yl), 4H-,6H-(1-pyrindin-1-yl), 2H-,3H-,4H-,6H-(1-pyrindin-1-yl),

7H-(pyrano[3,4-b]pyrol-1-yl), 5H-(pyrano[3,4-b]pyrol-1-yl), 3aH-(pyrano[3,4-b]pyrol-1-yl), 4H-,5H-,7H-(pyrano[3,4-b]pyrol-1-yl), 5H-,7H-,7aH-(pyrano[3,4-b]pyrol-1-yl), 7H-,3aH-,7aH-(pyrano[3,4-b]pyrol-1-yl), 4H-,5H-,3aH-(pyrano[3,4-b]pyrol-1-yl), 2H-,3aH-(pyrano[3,4-b]pyrol-1-yl), 2H-,7H-,3aH-,7aH-(pyrano[3,4-b]pyrol-1-yl), 2H-,4H-,5H-,3aH-(pyrano[3,4-b]pyrol-1-yl), 2H-,3H-,7H-(pyrano[3,4-b]pyrol-1-yl), 2H-,3H-,5H-(pyrano[3,4-b]pyrol-1-yl), 2H-,3H-,3aH-(pyrano[3,4-b]pyrol-1-yl), 2H-,3H-,4H-,5H-,7H-(pyrano[3,4-b]pyrol-1-yl), 2H-,3H-,5H-,7H-,7aH-(pyrano[3,4-b]pyrol-1-yl), 2H-,3H-,7H-,3aH-,7aH-(pyrano[3,4-b]pyrol-1-yl), 2H-,3H-,4H-,5H-,3aH-(pyrano[3,4-b]pyrol-1-yl),

isoindazol-1-yl, 6H-,7H-isoindazol-1-yl, 4H-,7H-isoindazol-1-yl, 4H-,5H-isoindazol-1-yl, 7H-,7aH-isoindazol-1-yl, 5H-,7aH-isoindazol-1-yl, 5H-,6H-isoindazol-1-yl, 4aH-,7aH-isoindazol-1-yl, 3aH-,6H-isoindazol-1-yl, 3aH-,4H-isoindazol-1-yl, 4H-,5H-,6H-,7H-isoindazol-1-yl, 5H-,6H-,7H-,7aH-isoindazol-1-yl, 6H-,7H-,3aH-,7aH-isoindazol-1-yl, 4H-, 7H-,3aH-,7aH-isoindazol-1-yl, 4H-,5H-,3aH-,7aH-isoindazol-1-yl, 4H-,5H-,6H-,3aH-isoindazol-1-yl,

2H-isoindazol-1-yl, 2H-,4aH-,7aH-isoindazol-1-yl, 2H-,3aH-,6H-isoindazol-1-yl, 2H-, 3aH-,4H-isoindazol-1-yl, 2H-,6H-,7H-,3aH-,7aH-isoindazol-1-yl, 2H-,4H-,7H-,3aH-,7aH-isoindazol-1-yl, 2H-,4H-,5H-,3aH-,7 aH-isoindazol-1-yl, 2H-,4H-,5H-,6H-,3aH-isoindazol-1-yl,

2H-,3H-isoindazol-1-yl, 2H-,3H-,6H-,7H-isoindazol-1-yl, 2H-,3H-,4H-,7H-isoindazol-1-yl, 2H-,3H-,4H-,5H-isoindazol-1-yl, 2H-,3H-,7H-,7aH-isoindazol-1-yl, 2H-,3H-, 5H-,7aH-isoindazol-1-yl, 2H-,3H-,5H-,6H-isoindazol-1-yl, 2H-,3H-,4aH-,7aH-isoindazol-1-yl, 2H-,3H-,3aH-,6H-isoindazol-1-yl, 2H-,3H-,3aH-,4H-isoindazol-1-yl, 2H-,3H-,4H-,5H-, 6H-,7H-isoindazol-1-yl, 2H-,3H-,5H-,6H-,7H-,7aH-isoindazol-1-yl, 2H-,3H-,6H-,7H-,3aH-, 7aH-isoindazol-1-yl, 2H-,3H-,4H-,7H-,3aH-,7 aH-isoindazol-1-yl, 2H-,3H-,4H-,5H-,3aH-, 7aH-isoindazol-1-yl, 2H-,3H-,4H-,5H-,6H-,3aH-isoindazol-1-yl,

isoindazol-2-yl, 4H-,5H-isoindazol-2-yl, 4H-,7H-isoindazol-2-yl, 1H-,3H-isoindazol-2-yl, 1H-,3H-isoindazol-2-yl, 1H-,3H-,4H-,5H-isoindazol-2-yl, 1H-,3H-,4H-,7H-isoindazol-2-yl, 1H-,3H-,4H-,4aH-,7aH-,7H-isoindazol-2-yl, 1H-,3H-,4aH-,7aH-isoindazol-2-yl, 1H-, 3H-,5H-,6H-isoindazol-2-yl, 3H-,4aH-isoindazol-2-yl, 3H-,5H-isoindazol-2-yl, 3H-,7H-isoindazol-2-yl, 3H-,4aH-,4H-,7aH-,7H-isoindazol-2-yl, 3H-,4aH-,6H-,7H-isoindazol-2-yl, 3H-,5H-,6H-,7H-isoindazol-2-yl, 4H-,4aH-,5H-isoindazol-2-yl, 1H-,3H-,4aH-,6H-,7H-,7aH-isoindazol-2-yl, 1H-,3H-,5H-,6H-,7H-,7aH-isoindazol-2-yl, 3H-,4H-,4aH-,5H-,6H-,7H-isoindazol-2-yl, 1H-,3H-,4H-,5H-,6H-,7H-isoindazol-2-yl, 4H-,5H-,6H-,7H-isoindazol-2-yl, 1H-,3H-,7H-,7aH-isoindazol-2-yl, 1H-,3H-,4H-,4aH-,5H-,6H-isoindazol-2-yl, 1H-,3H-,4H-, 4aH-,5H-,7H-isoindazol-2-yl, 1H-,7aH-isoindazol-2-yl, 1H-,4H-isoindazol-2-yl, 1H-,4H-, 7H-,7aH-isoindazol-2-yl, 1H-,6H-,7H-,7aH-isoindazol-2-yl, 6H-,7H-isoindazol-2-yl, 1H-, 3H-,6H-,7H-isoindazol-2-yl, 1H-,3H-,4aH-,6H-isoindazol-2-yl, 1H-,4H-,5H-,6H-,7H-,7aH-isoindazol-2-yl,

indoxazin-2-yl, 3H-indoxazin-2-yl, 3H-,4H-,5H-indoxazin-2-yl, 3H-,4H-,7H-indoxazin-2-yl, 3H-,4H-,4aH-,7aH-,7H-indoxazin-2-yl, 3H-,4aH-,7aH-indoxazin-2-yl, 3H-, 5H-,6H-indoxazin-2-yl, 3H-,4aH-,6H-,7H-,7aH-indoxazin-2-yl, 3H-,5H-,6H-,7H-,7aH-indoxazin-2-yl, 3H-,4H-,5H-,6H-,7H-indoxazin-2-yl, 3H-,7H-,7aH-indoxazin-2-yl, 3H-,4H-, 4aH-,5H-,6H-indoxazin-2-yl, 3H-,4H-,4aH-,5H-,7H-indoxazin-2-yl, 7aH-indoxazin-2-yl, 4H-indoxazin-2-yl, 4H-,7H-,7aH-indoxazin-2-yl, 6H-,7H-,7aH-indoxazin-2-yl, 3H-,6H-,7H-indoxazin-2-yl, 3H-,4aH-,6H-indoxazin-2-yl, 4H-,5H-,6H-,7H-,7aH-indoxazin-2-yl,

benzoxazol-3-yl, 4H-,5H-benzoxazol-3-yl, 4H-,7H-benzoxazol-3-yl, 4H-,4aH-,7aH-, 7H-benzoxazol-3-yl, 4aH-,7aH-benzoxazol-3-yl, 5H-,6H-benzoxazol-3-yl, 4aH-,6H-,7H-, 7aH-benzoxazol-3-yl, 5H-,6H-,7H-,7aH-benzoxazol-3-yl, 4H-,5H-,6H-,7H-benzoxazol-3-yl, 7H-,7aH-benzoxazol-3-yl, 4H-,4aH-,5H-,6H-benzoxazol-3-yl, 4H-,4aH-,5H-,7H-benzoxazol-3-yl, 3H-,6H-,7H-benzoxazol-3-yl, 4aH-,6H-benzoxazol-3-yl,

anthran-1-yl, 4aH-,7 aH-anthran-1-yl, 3aH-,6H-anthran-1-yl, 3 aH-,4H-anthran-1-yl, 6H-,7H-,3aH-,7aH-anthran-1-yl, 4H-,7H-,3aH-,7aH-anthran-1-yl, 4H-,5H-,3aH-,7aH-anthran-1-yl, 4H-,5H-,6H-,3aH-anthran-1-yl, 3H-anthran-1-yl, 3H-,6H-,7H-anthran-1-yl, 3H-,4H-,7H-anthran-1-yl, 3H-,4H-,5H-anthran-1-yl, 3H-,7H-,7aH-anthran-1-yl, 3H-,5H-,7aH-anthran-1-yl, 3H-,5H-,6H-anthran-1-yl, 3H-,4aH-,7aH-anthran-1-yl, 3H-,3aH-,6H-anthran-1-yl, 3H-, 3aH-,4H-anthran-1-yl, 3H-,4H-,5H-,6H-,7H-anthran-1-yl, 3H-,5H-,6H-,7H-,7aH-anthran-1-yl, 3H-,6H-,7H-,3aH-,7aH-anthran-1-yl, 3H-,4H-,7H-,3aH-,7aH-anthran-1-yl, 3H-,4H-,5H-, 3aH-,7aH-anthran-1-yl, 3H-,4H-,5H-,6H-,3 aH-anthran-1-yl,

5H-quinol-1-yl, 2H-quinol-1-yl, 4H-,5H-,6H-,7H-,8H-quinol-1-yl, 2H-,5H-,5aH-,6H-, 7H-,8H-,8aH-quinol-1-yl, 2H-,3H-,5H-,6H-,7H-,8H-,8aH-quinol-1-yl, 4H-,5H-,5Ha-,6H-, 7H-,8H-,8aH-quinol-1-yl, 2H-,3H-,4H-,5H-,6H-,7H-,8H-quinol-1-yl, 8 aH-quinol-1-yl, 7H-, 8H-,8 aH-quinol-1-yl, 5H-,8H-,8 aH-quinol-1-yl, 5H-,6H-,8 aH-quinol-1-yl, 5H-,6H-quinol-1-yl, 5H-,6H-,7H-,8H-,8aH-quinol-1-yl, 2H-,7H-,8H-quinol-1-yl, 2H-,5H-,8H-quinol-1-yl, 2H-, 5H-,6H-quinol-1-yl, 2H-,5H-,6H-,7H-,8H-quinol-1-yl, 4H-,7H-,8H-quinol-1-yl, 4H-,5H-,6H-quinol-1-yl, 4H-quinol-1-yl, 2H-,3H-,7H-,8H-,8 aH-quinol-1-yl, 2H-,3H-,5H-,8H-,8 aH-quinol-1-yl, 2H-,3H-,5H-,6H-,8 aH-quinol-1-yl, 2H-,3H-,5H-,6H-,7H-quinol-1-yl, 2H-,3H-, 5H-quinol-1-yl, 2H-,3H-,7H-quinol-1-yl, 2H-,3H-,8 aH-quinol-1-yl, 2H-,6H-,7H-,8H-,8 aH-quinol-1-yl, 2H-,5H-,5 aH-,8H-,8 aH-quinol-1-yl, 2H-,5H-,5 aH-,6H-quinol-1-yl, 2H-,5H-,5 aH-, 6H-,7H-quinol-1-yl, 2H-,5H-,6H-,7H-,8H-quinol-1-yl, 2H-,7H-,8H-quinol-1-yl, 2H-,5H-,6H-quinol-1-yl, 2H-,5 H-,8 H-quinol-1-yl, 2H-,6H-,7 H-quinol-1-yl, 2H-,5aH-,8 aH-quinol-1-yl, 2H-,6H-,8aH-quinol-1-yl, 2H-,5aH-,7H-quinol-1-yl, 4H-,6H-,7H-,8H-,8aH-quinol-1-yl, 4H-, 5H-,5 aH-,8H-,8 aH-quinol-1-yl, 4H-,5H-,5 aH-,6H-quinol-1-yl, 4H-,5H-,5 aH-,6H-,7H-quinol-1-yl, 4H-,5H-,6H-,7H-,8H-quinol-1-yl, 4H-,7H-,8H-quinol-1-yl, 4H-,5H-,6H-quinol-1-yl, 4H-, 5H-,8H-quinol-1-yl, 4H-,6H-,7H-quinol-1-yl, 4H-,5 aH-,8 aH-quinol-1-yl, 2H-,3H-,4H-,7H-, 8H-quinol-1-yl, 2H-,3H-,4H-,5H-,8H-quinol-1-yl, 2H-,3H-,4H-,5H-,6H-quinol-1-yl, 2H-,3H-, 4H-,6H-,7H-quinol-1-yl, 2H-,3H-,4H-,5 aH-,8 aH-quinol-1-yl, 2H-,3H-,4H-,5H-,8H-quinol-1-yl, 2H-,3H-,4H-,5aH-,7H-quinol-1-yl, 2H-,3H-,4H-,6H-,8aH-quinol-1-yl, 2H-,3H-,4H-,5H-, 6H-quinol-1-yl, 2H-,3H-,4H-,7H-,8H-quinol-1-yl, 4H-,6H-,8 aH-quinol-1-yl, 4H-,5aH-,7H-quinol-1-yl,

1H-isoquinol-2-yl, 1H-,5H-,6H-isoquinol-2-yl, 1H-,5H-,8H-isoquinol-2-yl, 1H-,7H-, 8H-isoquinol-2-yl, 1H-,5H-,6H-,7H-,8H-isoquinol-2-yl,

3H-isoquinol-2-yl, 3H-,5H-,6H-isoquinol-2-yl, 3H-,5H-,8H-isoquinol-2-yl, 3H-,7H-, 8H-isoquinol-2-yl, 3H-,5H-,6H-,7H-,8H-isoquinol-2-yl,

5aH-isoquinol-2-yl, 5H-,5aH-,6H-isoquinol-2-yl, 5H-,5aH-,8H-isoquinol-2-yl, 5aH-, 7H-,8H-isoquinol-2-yl, 6H-,7H-,8H-isoquinol-2-yl, 8H-isoquinol-2-yl, 6H-isoquinol-2-yl, 5H-,5aH-,6H-,7H-,8H-isoquinol-2-yl,

3H-,4H-,5aH-isoquinol-2-yl, 3H-,4H-,6H-isoquinol-2-yl, 3H-,4H-,6H-,7H-,8H-isoquinol-2-yl, 3H-,4H-,5aH-,7H-,8H-isoquinol-2-yl, 3H-,4H-,5H-,5aH-,8H-isoquinol-2-yl, 3H-,4H-,5H-,5aH-,6H-isoquinol-2-yl, 3H-,4H-,8H-isoquinol-2-yl, 3H-,4H-,5H-,5aH-,6H-,7H-, 8H-isoquinol-2-yl,

1H-,3H-,8aH-isoquinol-2-yl, 1H-,3H-,7H-isoquinol-2-yl, 1H-,3H-,5H-isoquinol-2-yl, 1H-,3H-,7H-,8H-,8aH-isoquinol-2-yl, 1H-,3H-,5H-,8H-,8aH-isoquinol-2-yl, 1H-,3H-,5H-,6H-, 8aH-isoquinol-2-yl, 1H-,3H-,5H-,6H-,7H-isoquinol-2-yl, 1H-,3H-,5H-,6H-,7H-,8H-,8aH-isoquinol-2-yl,

1H-,3H-,4H-,7H-,8H-isoquinol-2-yl, 1H-,3H-,4H-,7H-,8H-isoquinol-2-yl, 1H-,3H-, 4H-,5H-,8H-isoquinol-2-yl, 1H-,3H-,4H-,5H-,6H-isoquinol-2-yl, 1H-,3H-,4H-,5H-,6H-,7H-, 8H-isoquinol-2-yl,

1H-,5aH-,8aH-isoquinol-2-yl, 1H-,5aH-,7H-isoquinol-2-yl, 1H-,5aH-,7H-,8H-,8aH-isoquinol-2-yl, 1H-,5H-,5aH-,8H-,8aH-isoquinol-2-yl, 1H-,5H-,5aH-,6H-,8aH-isoquinol-2-yl, 1H-,5H-,5aH-,6H-,7H-isoquinol-2-yl, 1H-,6H-,8aH-isoquinol-2-yl, 1H-,8H-,8aH-isoquinol-2-yl, 1H-,6H-,7H-isoquinol-2-yl, 1H-,5H-,5aH-,6H-,7H-,8H-,8aH-isoquinol-2-yl,

1H-,3H-,4H-,5H-,5aH-,6H-,7H-,8H-,8aH-isoquinol-2-yl, 1H-,3H-,4H-,6H-,7H-,8H-, 8aH-isoquinol-2-yl, 1H-,3H-,4H-,5aH-,7H-,8H-,8aH-isoquinol-2-yl, 1H-,3H-,4H-,5H-,5aH-, 8H-,8aH-isoquinol-2-yl, 1H-,3H-,4H-,5H-,5aH-,6H-,8aH-isoquinol-2-yl, 1H-,3H-,4H-,5H-, 5aH-,6H-,7H-isoquinol-2-yl, 1H-,3H-,4H-,5H-,6H-,7H-,8H-isoquinol-2-yl, 1H-,3H-,4H-,7H-, 8H-isoquinol-2-yl, 1H-,3H-,4H-,5H-,8H-isoquinol-2-yl, 1H-,3H-,4H-,6H-,7H-isoquinol-2-yl, 1H-,3H-,4H-,5H-,6H-isoquinol-2-yl, 1H-,3H-,4H-,5aH-,8aH-isoquinol-2-yl, 1H-,3H-,4H-,8H-, 8aH-isoquinol-2-yl, 1H-,3H-,4H-,6H-,8aH-isoquinol-2-yl, 1H-,3H-,4H-,5aH-,7H-isoquinol-2-yl, 1H-,3H-,4H-,5H-,5aH-isoquinol-2-yl,

5H-cinnol-1-yl, 2H-cinnol-1-yl, 4H-,5H-,6H-,7H-,8H-cinnol-1-yl, 2H-,5H-,5aH-,6H-, 7H-,8H-,8aH-cinnol-1-yl, 2H-,3H-,5H-,6H-,7H-,8H-,8aH-cinnol-1-yl, 4H-,5H-,5Ha-,6H-, 7H-,8H-,8aH-cinnol-1-yl, 2H-,3H-,4H-,5H-,6H-,7H-,8H-cinnol-1-yl, 8aH-cinnol-1-yl, 7H-, 8H-,8aH-cinnol-1-yl, 5H-,8H-,8aH-cinnol-1-yl, 5H-,6H-,8aH-cinnol-1-yl, 5H-,6H-cinnol-1-yl, 5H-,6H-,7H-,8H-,8aH-cinnol-1-yl, 2H-,7H-,8H-cinnol-1-yl, 2H-,5H-,8H-cinnol-1-yl, 2H-, 5H-,6H-cinnol-1-yl, 2H-,5H-,6H-,7H-,8H-cinnol-1-yl, 4H-,7H-,8H-cinnol-1-yl, 4H-,5H-,6H-cinnol-1-yl, 4H-cinnol-1-yl, 2H-,3H-,7H-,8H-,8aH-cinnol-1-yl, 2H-,3H-,5H-,8H-,8aH-cinnol-1-yl, 2H-,3H-,5H-,6H-,8aH-cinnol-1-yl, 2H-,3H-,5H-,6H-,7H-cinnol-1-yl, 2H-,3H-,5H-cinnol-1-yl, 2H-,3H-,7H-cinnol-1-yl, 2H-,3H-,8aH-cinnol-1-yl, 2H-,6H-,7H-,8H-,8aH-cinnol-1-yl, 2H-,5H-,5aH-,8H-,8aH-cinnol-1-yl, 2H-,5H-,5aH-,6H-cinnol-1-yl, 2H-,5H-,5aH-,6H-, 7H-cinnol-1-yl, 2H-,5H-,6H-,7H-,8H-cinnol-1-yl, 2H-,7H-,8H-cinnol-1-yl, 2H-,5H-,6H-cinnol-1-yl, 2H-,5H-,8H-cinnol-1-yl, 2H-,6H-,7H-cinnol-1-yl, 2H-,5aH-,8aH-cinnol-1-yl, 2H-, 6H-,8aH-cinnol-1-yl, 2H-,5aH-,7H-cinnol-1-yl, 4H-,6H-,7H-,8H-,8aH-cinnol-1-yl, 4H-,5H-, 5aH-,8H-,8aH-cinnol-1-yl, 4H-,5H-,5aH-,6H-cinnol-1-yl, 4H-,5H-,5aH-,6H-,7H-cinnol-1-yl, 4H-,5H-,6H-,7H-,8H-cinnol-1-yl, 4H-,7H-,8H-cinnol-1-yl, 4H-,5H-,6H-cinnol-1-yl, 4H-,5H-, 8H-cinnol-1-yl, 4H-,6H-,7H-cinnol-1-yl, 4H-,5aH-,8aH-cinnol-1-yl, 2H-,3H-,4H-,7H-,8H-cinnol-1-yl, 2H-,3H-,4H-,5H-,8H-cinnol-1-yl, 2H-,3H-,4H-,5H-,6H-cinnol-1-yl, 2H-,3H-,4H-, 6H-,7H-cinnol-1-yl, 2H-,3H-,4H-,5aH-,8aH-cinnol-1-yl, 2H-,3H-,4H-,5H-,8H-cinnol-1-yl, 2H-,3H-,4H-,5aH-,7H-cinnol-1-yl, 2H-,3H-,4H-,6H-,8aH-cinnol-1-yl, 2H-,3H-,4H-,5H-,6H-cinnol-1-yl, 2H-,3H-,4H-,7H-,8H-cinnol-1-yl, 4H-,6H-,8aH-cinnol-1-yl, 4H-,5aH-,7H-cinnol-1-yl,

1H-cinnol-2-yl, 1H-,5H-,6H-cinnol-2-yl, 1H-,5H-,8H-cinnol-2-yl, 1H-,7H-,8H-cinnol-2-yl, 1H-,5H-,6H-,7H-,8H-cinnol-2-yl,

3H-cinnol-2-yl, 3H-,5H-,6H-cinnol-2-yl, 3H-,5H-,8H-cinnol-2-yl, 3H-,7H-,8H-cinnol-2-yl, 3H-,5H-,6H-,7H-,8H-cinnol-2-yl,

5aH-cinnol-2-yl, 5H-,5aH-,6H-cinnol-2-yl, 5H-,5aH-,8H-cinnol-2-yl, 5aH-,7H-,8H-cinnol-2-yl, 6H-,7H-,8H-cinnol-2-yl, 8H-cinnol-2-yl, 6H-cinnol-2-yl, 5H-,5aH-,6H-,7H-,8H-cinnol-2-yl,

3H-,4H-,5aH-cinnol-2-yl, 3H-,4H-,6H-cinnol-2-yl, 3H-,4H-,6H-,7H-,8H-cinnol-2-yl, 3H-,4H-,5aH-,7H-,8H-cinnol-2-yl, 3H-,4H-,5H-,5aH-,8H-cinnol-2-yl, 3H-,4H-,5H-,5aH-,6H-cinnol-2-yl, 3H-,4H-,8H-cinnol-2-yl, 3H-,4H-,5H-,5aH-,6H-,7H-,8H-cinnol-2-yl,

1H-,3H-,8aH-cinnol-2-yl, 1H-,3H-,7H-cinnol-2-yl, 1H-,3H-,5H-cinnol-2-yl, 1H-,3H-, 7H-,8H-,8aH-cinnol-2-yl, 1H-,3H-,5H-,8H-,8aH-cinnol-2-yl, 1H-,3H-,5H-,6H-,8aH-cinnol-2-yl, 1H-,3H-,5H-,6H-,7H-cinnol-2-yl, 1H-,3H-,5H-,6H-,7H-,8H-,8aH-cinnol-2-yl,

1H-,3H-,4H-,7H-,8H-cinnol-2-yl, 1H-,3H-,4H-,7H-,8H-cinnol-2-yl, 1H-,3H-,4H-,5H-, 8H-cinnol-2-yl, 1H-,3H-,4H-,5H-,6H-cinnol-2-yl, 1H-,3H-,4H-,5H-,6H-,7H-,8H-cinnol-2-yl,

1H-,5aH-,8aH-cinnol-2-yl, 1H-,5aH-,7H-cinnol-2-yl, 1H-,5aH-,7H-,8H-,8aH-cinnol-2-yl, 1H-,5H-,5aH-,8H-,8aH-cinnol-2-yl, 1H-,5H-,5aH-,6H-,8aH-cinnol-2-yl, 1H-,5H-,5aH-, 6H-,7H-cinnol-2-yl, 1H-,6H-,8aH-cinnol-2-yl, 1H-,8H-,8aH-cinnol-2-yl, 1H-,6H-,7H-cinnol-2-yl, 1H-,5H-,5aH-,6H-,7H-,8H-,8aH-cinnol-2-yl,

1H-,3H-,4H-,5H-,5aH-,6H-,7H-,8H-,8aH-cinnol-2-yl, 1H-,3H-,4H-,6H-,7H-,8H-,8aH-cinnol-2-yl, 1H-,3H-,4H-,5aH-,7H-,8H-,8aH-cinnol-2-yl, 1H-,3H-,4H-,5H-,5aH-,8H-,8aH-cinnol-2-yl, 1H-,3H-,4H-,5H-,5aH-,6H-,8aH-cinnol-2-yl, 1H-,3H-,4H-,5H-,5aH-,6H-,7H-cinnol-2-yl, 1H-,3H-,4H-,5H-,6H-,7H-,8H-cinnol-2-yl, 1H-,3H-,4H-,7H-,8H-cinnol-2-yl, 1H-, 3H-,4H-,5H-,8H-cinnol-2-yl, 1H-,3H-,4H-,6H-,7H-cinnol-2-yl, 1H-,3H-,4H-,5H-,6H-cinnol-2-yl, 1H-,3H-,4H-,5aH-,8aH-cinnol-2-yl, 1H-,3H-,4H-,8H-,8aH-cinnol-2-yl, 1H-,3H-,4H-, 6H-,8aH-cinnol-2-yl, 1H-,3H-,4H-,5aH-,7H-cinnol-2-yl, 1H-,3H-,4H-,5H-,5aH-cinnol-2-yl,

5H-quinazol-1-yl, 2H-quinazol-1-yl, 4H-,5H-,6H-,7H-,8H-quinazol-1-yl, 2H-,5H-, 5aH-,6H-,7H-,8H-,8aH-quinazol-1-yl, 2H-,3H-,5H-,6H-,7H-,8H-,8aH-quinazol-1-yl, 4H-, 5H-,5Ha-,6H-,7H-,8H-,8 aH-quinazol-1-yl, 2H-,3H-,4H-,5H-,6H-,7H-,8H-quinazol-1-yl, 8 aH-quinazol-1-yl, 7H-,8H-,8aH-quinazol-1-yl, 5H-,8H-,8 aH-quinazol-1-yl, 5H-,6H-,8aH-quinazol-1-yl, 5H-,6H-quinazol-1-yl, 5H-,6H-,7H-,8H-,8aH-quinazol-1-yl, 2H-,7H-,8H-quinazol-1-yl, 2H-,5H-,8H-quinazol-1-yl, 2H-,5H-,6H-quinazol-1-yl, 2H-,5H-,6H-,7H-,8H-quinazol-1-yl, 4H-,7H-,8H-quinazol-1-yl, 4H-,5H-,6H-quinazol-1-yl, 4H-quinazol-1-yl, 2H-, 3H-,7H-,8H-,8aH-quinazol-1-yl, 2H-,3H-,5H-,8H-,8aH-quinazol-1-yl, 2H-,3H-,5H-,6H-, 8aH-quinazol-1-yl, 2H-,3H-,5H-,6H-,7H-quinazol-1-yl, 2H-,3H-,5H-quinazol-1-yl, 2H-,3H-, 7H-quinazol-1-yl, 2H-,3H-,8 aH-quinazol-1-yl, 2H-,6H-,7H-,8H-,8aH-quinazol-1-yl, 2H-,5H-, 5aH-,8H-,8aH-quinazol-1-yl, 2H-,5H-,5 aH-,6H-quinazol-1-yl, 2H-,5H-,5aH-,6H-,7H-quinazol-1-yl, 2H-,5H-,6H-,7H-,8H-quinazol-1-yl, 2H-,7H-,8H-quinazol-1-yl, 2H-,5H-,6H-quinazol-1-yl, 2H-,5H-,8H-quinazol-1-yl, 2H-,6H-,7H-quinazol-1-yl, 2H-,5 aH-,8 aH-quinazol-1-yl, 2H-,6H-,8 aH-quinazol-1-yl, 2H-,5aH-,7H-quinazol-1-yl, 4H-,6H-,7H-,8H-, 8 aH-quinazol-1-yl, 4H-,5H-,5 aH-,8 H-,8 aH-quinazol-1-yl, 4H-,5H-,5aH-,6H-quinazol-1-yl, 4H-,5H-,5aH-,6H-,7H-quinazol-1-yl, 4H-,5H-,6H-,7H-,8H-quinazol-1-yl, 4H-,7H-,8H-quinazol-1-yl, 4H-,5H-,6H-quinazol-1-yl, 4H-,5H-,8H-quinazol-1-yl, 4H-,6H-,7H-quinazol-1-yl, 4H-,5aH-,8aH-quinazol-1-yl, 2H-,3H-,4H-,7H-,8H-quinazol-1-yl, 2H-,3H-,4H-,5H-,8H-quinazol-1-yl, 2H-,3H-,4H-,5H-,6H-quinazol-1-yl, 2H-,3H-,4H-,6H-,7H-quinazol-1-yl, 2H-, 3H-,4H-,5aH-,8aH-quinazol-1-yl, 2H-,3H-,4H-,5H-,8H-quinazol-1-yl, 2H-,3H-,4H-,5aH-, 7H-quinazol-1-yl, 2H-,3H-,4H-,6H-,8 aH-quinazol-1-yl, 2H-,3H-,4H-,5H-,6H-quinazol-1-yl,

2H-,3H-,4H-,7H-,8H-quinazol-1-yl, 4H-,6H-,8aH-quinazol-1-yl, 4H-,5aH-,7H-quinazol-1-yl, 2H-,4H-,5H-,5aH-,6H-,7H-,8H-quinazol-3-yl, 2H-,4H-,6H-,7H-,8H-quinazol-3-yl, 2H-,4H-,5aH-,7H-,8H-quinazol-3-yl, 2H-,4H-,5H-,5aH-,8H-quinazol-3-yl, 2H-,4H-,5H-,5aH-, 6H-quinazol-3-yl, 2H-,4H-,8H-quinazol-3-yl, 2H-,4H-,6H-quinazol-3-yl, 2H-,4H-,5aH-quinazol-3-yl, 4H-,5H-,5aH-,6H-,7H-,8H-,8aH-quinazol-3-yl, 4H-,6H-,7H-,8H-,8aH-quinazol-3-yl, 4H-,5aH-,7H-,8H-,8aH-quinazol-3-yl, 4H-,5H-,5aH-,8H-,8aH-quinazol-3-yl, H-,5H-,5aH-,6H-,8aH-quinazol-3-yl, 4H-,5H-,5aH-,6H-,7H-quinazol-3-yl, 4H-,6H-,7H-quinazol-3-yl, 4H-,5aH-,7H-quinazol-3-yl, 4H-,5H-,5aH-quinazol-3-yl, 4H-,6H-,8aH-quinazol-3-yl, 4H-,7H-,8aH-quinazol-3-yl, 4H-,5aH-,8aH-quinazol-3-yl, 2H-,5H-,6H-,7H-, 8H-quinazol-3-yl, 2H-,7H-,8H-quinazol-3-yl, 2H-,5H-,8H-quinazol-3-yl, 2H-,5H-,6H-quinazol-3-yl, 2H-quinazol-3-yl, 5H-,6H-,7H-,8H-,8aH-quinazol-3-yl, 7H-,8H-,8aH-quinazol-3-yl, 5H-,8H-,8aH-quinazol-3-yl, 5H-,6H-,8aH-quinazol-3-yl, 5H-,6H-,7H-quinazol-3-yl, 6H-quinazol-3-yl, 5H-quinazol-3-yl, 8aH-quinazol-3-yl, 4H-,5H-,6H-,7H-,8H-quinazol-3-yl, 4H-,7H-,8H-quinazol-3-yl, 4H-,5H-,8H-quinazol-3-yl, 4H-,5H-,6H-quinazol-3-yl, 4H-quinazol-3-yl, 1H-,2H-,5H-,6H-,7H-,8H-,8aH-quinazol-3-yl, 1H-,2H-,4H-,5H-,6H-, 7H-,8H-quinazol-3-yl, 1H-,2H-,7H-,8H-,8aH-quinazol-3-yl, 1H-,2H-,5H-,8H-,8aH-quinazol-3-yl, 1H-,2H-,5H-,6H-,8aH-quinazol-3-yl, 1H-,2H-,5H-,6H-,7H-quinazol-3-yl, 1H-,2H-,5H-quinazol-3-yl, 1H-,2H-,7H-quinazol-3-yl, 1H-,2H-,8aH-quinazol-3-yl, 1H-,2H-,4H-,7H-,8H-quinazol-3-yl, 1H-,2H-,4H-,5H-,8H-quinazol-3-yl, 1H-,2H-,4H-,5H-,6H-quinazol-3-yl, 1H-, 2H-,4H-,6H-,7H-quinazol-3-yl, 1H-,2H-,4H-,5aH-,8aH-quinazol-3-yl, 1H-,2H-,4H-,5H-,8H-quinazol-3-yl, 1H-,2H-,4H-,5aH-,7H-quinazol-3-yl, 1H-,2H-,4H-,6H-,8aH-quinazol-3-yl, 1H-, 2H-,4H-,5H-,6H-quinazol-3-yl, 1H-,2H-,4H-,7H-,8H-quinazol-3-yl,

5H-naphthyridin-1-yl, 2H-naphthyridin-1-yl, 4H-,5H-,6H-,7H-,8H-naphthyridin-1-yl, 2H-,5H-,5aH-,6H-,7H-,8H-,8aH-naphthyridin-1-yl, 2H-,3H-,5H-,6H-,7H-,8H-,8 aH-naphthyridin-1-yl, 4H-,5H-,5Ha-,6H-,7H-,8H-,8 aH-naphthyridin-1-yl, 2H-,3H-,4H-,5H-,6H-, 7H-,8H-naphthyridin-1-yl, 8 aH-naphthyridin-1-yl, 7H-,8H-,8 aH-naphthyridin-1-yl, 5H-,8H-, 8 aH-naphthyridin-1-yl, 5H-,6H-,8 aH-naphthyridin-1-yl, 5H-,6H-naphthyridin-1-yl, 5H-,6H-, 7H-,8H-,8 aH-naphthyridin-1-yl, 2H-,7H-,8H-naphthyridin-1-yl, 2H-,5H-,8H-naphthyridin-1-yl, 2H-,5H-,6H-naphthyridin-1-yl, 2H-,5H-,6H-,7H-,8H-naphthyridin-1-yl, 4H-,7H-,8H-naphthyridin-1-yl, 4H-,5H-,6H-naphthyridin-1-yl, 4H-naphthyridin-1-yl, 2H-,3H-,7H-,8H-, 8 aH-naphthyridin-1-yl, 2H-,3H-,5H-,8H-,8 aH-naphthyridin-1-yl, 2H-,3H-,5H-,6H-,8 aH-naphthyridin-1-yl, 2H-,3H-,5H-,6H-,7H-naphthyridin-1-yl, 2H-,3H-,5H-naphthyridin-1-yl, 2H-,3H-,7H-naphthyridin-1-yl, 2H-,3H-,8 aH-naphthyridin-1-yl, 2H-,6H-,7H-,8H-,8 aH-naphthyridin-1-yl, 2H-,5H-,5aH-,8H-,8 aH-naphthyridin-1-yl, 2H-,5H-,5aH-,6H-naphthyridin-1-yl, 2H-,5H-,5aH-,6H-,7H-naphthyridin-1-yl, 2H-,5H-,6H-,7H-,8H-naphthyridin-1-yl, 2H-, 7H-,8H-naphthyridin-1-yl, 2H-,5H-,6H-naphthyridin-1-yl, 2H-,5H-,8H-naphthyridin-1-yl, 2H-,6H-,7H-naphthyridin-1-yl, 2H-,5 aH-,8 aH-naphthyridin-1-yl, 2H-,6H-,8 aH-naphthyridin-1-yl, 2H-,5aH-,7H-naphthyridin-1-yl, 4H-,6H-,7H-,8H-,8 aH-naphthyridin-1-yl, 4H-,5H-,5aH-, 8H-,8aH-naphthyridin-1-yl, 4H-,5H-,5aH-,6H-naphthyridin-1-yl, 4H-,5H-,5aH-,6H-,7H-naphthyridin-1-yl, 4H-,5H-,6H-,7H-,8H-naphthyridin-1-yl, 4H-,7H-,8H-naphthyridin-1-yl, 4H-,5H-,6H-naphthyridin-1-yl, 4H-,5H-,8H-naphthyridin-1-yl, 4H-,6H-,7H-naphthyridin-1-yl, 4H-,5aH-,8 aH-naphthyridin-1-yl, 2H-,3H-,4H-,7H-,8H-naphthyridin-1-yl, 2H-,3H-,4H-, 5H-,8H-naphthyridin-1-yl, 2H-,3H-,4H-,5H-,6H-naphthyridin-1-yl, 2H-,3H-,4H-,6H-,7H-naphthyridin-1-yl, 2H-,3H-,4H-,5aH-,8 aH-naphthyridin-1-yl, 2H-,3H-,4H-,5H-,8H-naphthyridin-1-yl, 2H-,3H-,4H-,5aH-,7H-naphthyridin-1-yl, 2H-,3H-,4H-,6H-,8 aH-naphthyridin-1-yl, 2H-,3H-,4H-,5H-,6H-naphthyridin-1-yl, 2H-,3H-,4H-,7H-,8H-naphthyridin-1-yl, 4H-,6H-,8 aH-naphthyridin-1-yl, 4H-,5aH-,7H-naphthyridin-1-yl,

5H-pyrido[3,4-b]pyridin-1-yl, 2H-pyrido[3,4-b]pyridin-1-yl, 4H-,5H-,6H-,7H-,8H-pyrido[3,4-b]pyridin-1-yl, 2H-,5H-,5 aH-,6H-,7H-,8H-,8 aH-pyrido[3,4-b]pyridin-1-yl, 2H-, 3H-,5H-,6H-,7H-,8H-,8aH-pyrido[3,4-b]pyridin-1-yl, 4H-,5H-,5Ha-,6H-,7H-,8H-,8aH-pyrido[3,4-b]pyridin-1-yl, 2H-,3H-,4H-,5H-,6H-,7H-,8H-pyrido[3,4-b]pyridin-1-yl, 8 aH-pyrido[3,4-b]pyridin-1-yl, 7H-,8H-,8 aH-pyrido[3,4-b]pyridin-1-yl, 5H-,8H-,8 aH-pyrido[3,4-b]pyridin-1-yl, 5 H-,6H-,8 aH-pyrido[3,4-b]pyridin-1-yl, 5 H-,6H-pyrido[3,4-b]pyridin-1-yl, 5H-,6H-,7H-,8H-,8aH-pyrido[3,4-b]pyridin-1-yl, 2H-,7H-,8H-pyrido[3,4-b]pyridin-1-yl, 2H-, 5H-,8H-pyrido[3,4-b]pyridin-1-yl, 2H-,5H-,6H-pyrido[3,4-b]pyridin-1-yl, 2H-,5H-,6H-,7H-, 8H-pyrido[3,4-b]pyridin-1-yl, 4H-,7H-,8H-pyrido[3,4-b]pyridin-1-yl, 4H-,5H-,6H-pyrido[3,4-b]pyridin-1-yl, 4H-pyrido[3,4-b]pyridin-1-yl, 2H-,3H-,7H-,8H-,8 aH-pyrido[3,4-b]pyridin-1-yl, 2H-,3H-,5H-,8H-,8 aH-pyrido[3,4-b]pyridin-1-yl, 2H-,3H-,5H-,6H-,8 aH-pyrido[3,4-b]pyridin-1-yl, 2H-,3H-,5H-,6H-,7H-pyrido[3,4-b]pyridin-1-yl, 2H-,3H-,5H-pyrido[3,4-b]pyridin-1-yl, 2H-,3H-,7H-pyrido[3,4-b]pyridin-1-yl, 2H-,3H-,8 aH-pyrido[3,4-b]pyridin-1-yl, 2H-,6H-,7H-,8H-,8 aH-pyrido[3,4-b]pyridin-1-yl, 2H-,5H-,5 aH-,8H-,8 aH-pyrido[3,4-b]pyridin-1-yl, 2H-,5H-,5 aH-,6H-pyrido[3,4-b]pyridin-1-yl, 2H-,5H-,5aH-,6H-, 7H-pyrido[3,4-b]pyridin-1-yl, 2H-,5H-,6H-,7H-,8H-pyrido[3,4-b]pyridin-1-yl, 2H-,7H-,8H-pyrido[3,4-b]pyridin-1-yl, 2H-,5H-,6H-pyrido[3,4-b]pyridin-1-yl, 2H-,5H-,8H-pyrido[3,4-b]pyridin-1-yl, 2H-,6H-,7H-pyrido[3,4-b]pyridin-1-yl, 2H-,5aH-,8 aH-pyrido[3,4-b]pyridin-1-yl, 2H-,6H-,8aH-pyrido[3,4-b]pyridin-1-yl, 2H-,5aH-,7H-pyrido[3,4-b]pyridin-1-yl, 4H-,6H-, 7H-,8H-,8aH-pyrido[3,4-b]pyridin-1-yl, 4H-,5 H-,5 aH-,8H-,8 aH-pyrido[3,4-b]pyridin-1-yl, 4H-,5H-,5 aH-,6H-pyrido[3,4-b]pyridin-1-yl, 4H-,5H-,5 aH-,6H-,7 H-pyrido[3,4-b]pyridin-1-yl, 4H-,5H-,6H-,7H-,8H-pyrido[3,4-b]pyridin-1-yl, 4H-,7H-,8H-pyrido[3,4-b]pyridin-1-yl, 4H-, 5H-,6H-pyrido[3,4-b]pyridin-1-yl, 4H-,5H-,8H-pyrido[3,4-b]pyridin-1-yl, 4H-,6H-,7H-pyrido[3,4-b]pyridin-1-yl, 4H-,5 aH-,8 aH-pyrido[3,4-b]pyridin-1-yl, 2H-,3H-,4H-,7H-,8H-pyrido[3,4-b]pyridin-1-yl, 2H-,3H-,4H-,5H-,8H-pyrido[3,4-b]pyridin-1-yl, 2H-,3H-,4H-,5H-, 6H-pyrido[3,4-b]pyridin-1-yl, 2H-,3H-,4H-,6H-,7H-pyrido[3,4-b]pyridin-1-yl, 2H-,3H-,4H-, 5aH-,8 aH-pyrido[3,4-b]pyridin-1-yl, 2H-,3H-,4H-,5H-,8H-pyrido[3,4-b]pyridin-1-yl, 2H-, 3H-,4H-,5aH-,7H-pyrido[3,4-b]pyridin-1-yl, 2H-,3H-,4H-,6H-,8 aH-pyrido[3,4-b]pyridin-1-yl, 2H-,3H-,4H-,5H-,6H-pyrido[3,4-b]pyridin-1-yl, 2H-,3H-,4H-,7H-,8H-pyrido[3,4-b]pyridin-1-yl, 4H-,6H-,8 aH-pyrido[3,4-b]pyridin-1-yl, 4H-,5aH-,7H-pyrido[3,4-b]pyridin-1-yl,

2H-,3H-,4H-,5H-,5aH-,6H-,8H-pyrido[3,4-b]pyridin-7-yl, 2H-,3H-,4H-,5H-,6H-,8H-pyrido[3,4-b]pyridin-7-yl, 2H-,3H-,4H-,5aH-,8H-pyrido[3,4-b]pyridin-7-yl, 2H-,3H-,4H-,5H-, 5aH-,6H-pyrido[3,4-b]pyridin-7-yl, 3H-,4H-,5H-,5aH-,6H-,8H-,8aH-pyrido[3,4-b]pyridin-7-yl, 3H-,4H-,6H-,8H-,8aH-pyrido[3,4-b]pyridin-7-yl, 3H-,4H-,5aH-,8H-,8aH-pyrido[3,4-b]pyridin-7-yl, 3H-,4H-,5H-,5aH-,6H-,8aH-pyrido[3,4-b]pyridin-7-yl, 3H-,4H-,5H-,5aH-,6H-pyrido[3,4-b]pyridin-7-yl, 3H-,4H-,6H-pyrido[3,4-b]pyridin-7-yl, 3H-,4H-,5 aH-pyrido[3,4-b]pyridin-7-yl, 4H-,5H-,5aH-,6H-,8H-pyrido[3,4-b]pyridin-7-yl, 4H-,6H-,8H-pyrido[3,4-b]pyridin-7-yl, 4H-,8H-pyrido[3,4-b]pyridin-7-yl, 3H-,4H-,5H-,6H-,8H-pyrido[3,4-b]pyridin-7-yl, 3H-,4H-,8H-pyrido[3,4-b]pyridin-7-yl, 2H-,5H-,5aH-,6H-,8H-pyrido[3,4-b]pyridin-7-yl, 2H-,6H-,8H-pyrido[3,4-b]pyridin-7-yl, 2H-,8H-pyrido[3,4-b]pyridin-7-yl, 3H-,5H-,6H-,8H-, 8aH-pyrido[3,4-b]pyridin-7-yl, 3H-,8H-,8 aH-pyrido[3,4-b]pyridin-7-yl, 3H-pyrido[3,4-b]pyridin-7-yl, 5H-,6H-,8H-pyrido[3,4-b]pyridin-7-yl, 8H-pyrido[3,4-b]pyridin-7-yl, 1H-,4H-, 5H-,6H-,8H-pyrido[3,4-b]pyridin-7-yl, 1H-,2H-,5H-,5aH-,6H-,8H-,8aH-pyrido[3,4-b]pyridin-7-yl, 1H-,2H-,3H-,5H-,6H-,8H-,8aH-pyrido[3,4-b]pyridin-7-yl, 1H-,4H-,5H-,5Ha-, 6H-,8H-,8aH-pyrido[3,4-b]pyridin-7-yl, 1H-,2H-,3H-,4H-,5H-,6H-,8H-pyrido[3,4-b]pyridin-7-yl, 1H-,8H-,8aH-pyrido[3,4-b]pyridin-7-yl, 1H-,5H-,6H-,8H-,8aH-pyrido[3,4-b]pyridin-7-yl, 1H-,2H-,8H-pyrido[3,4-b]pyridin-7-yl, 1H-,2H-,5H-,6H-,8H-pyrido[3,4-b]pyridin-7-yl, 1H-,4H-,8H-pyrido[3,4-b]pyridin-7-yl, 1H-,2H-,3H-,8H-,8aH-pyrido[3,4-b]pyridin-7-yl, 1H-, 2H-,3H-,5H-,8H-,8aH-pyrido[3,4-b]pyridin-7-yl, 1H-,2H-,3H-,5H-,6H-pyrido[3,4-b]pyridin-7-yl, 1H-,2H-,3H-pyrido[3,4-b]pyridin-7-yl, 1H-,2H-,6H-,8H-,8aH-pyrido[3,4-b]pyridin-7-yl, 1H-,2H-,5H-,5aH-,6H-pyrido[3,4-b]pyridin-7-yl, 1H-,2H-,5H-,6H-,8H-pyrido[3,4-b]pyridin-7-yl, 1H-,2H-,8H-pyrido[3,4-b]pyridin-7-yl, 1H-,2H-,6H-pyrido[3,4-b]pyridin-7-yl, 1H-,2H-, 5aH-pyrido[3,4-b]pyridin-7-yl, 1H-,4H-,6H-,8H-,8aH-pyrido[3,4-b]pyridin-7-yl, 1H-,4H-, 5H-,5aH-,6H-pyrido[3,4-b]pyridin-7-yl, 1H-,4H-,5H-,6H-,8H-pyrido[3,4-b]pyridin-7-yl, 1H-, 4H-,8H-pyrido[3,4-b]pyridin-7-yl, 1H-,4H-,6H-pyrido[3,4-b]pyridin-7-yl, 1H-,2H-,3H-,4H-, 8H-pyrido[3,4-b]pyridin-7-yl, 1H-,2H-,3H-,4H-,6H-pyrido[3,4-b]pyridin-7-yl, 1H-,2H-,3H-, 4H-,5aH-,8aH-pyrido[3,4-b]pyridin-7-yl, 1H-,2H-,3H-,4H-,5aH-pyrido[3,4-b]pyridin-7-yl, 1H-,2H-,3H-,4H-,8H-pyrido[3,4-b]pyridin-7-yl, 1H-,4H-,5aH-pyrido[3,4-b]pyridin-7-yl,

5H-pyrido[3,2-b]pyridin-1-yl, 2H-pyrido[3,2-b]pyridin-1-yl, 4H-,5H-,6H-,7H-,8H-pyrido[3,2-b]pyridin-1-yl, 2H-,5H-,5 aH-,6H-,7H-,8H-,8 aH-pyrido[3,2-b]pyridin-1-yl, 2H-, 3H-,5H-,6H-,7H-,8H-,8aH-pyrido[3,2-b]pyridin-1-yl, 4H-,5H-,5Ha-,6H-,7H-,8H-,8aH-pyrido[3,2-b]pyridin-1-yl, 2H-,3H-,4H-,5H-,6H-,7H-,8H-pyrido[3,2-b]pyridin-1-yl, 8 aH-pyrido[3,2-b]pyridin-1-yl, 7H-,8H-,8 aH-pyrido[3,2-b]pyridin-1-yl, 5H-,8H-,8 aH-pyrido[3,2-b]pyridin-1-yl, 5 H-,6H-,8 aH-pyrido[3,2-b]pyridin-1-yl, 5 H-,6H-pyrido[3,2-b]pyridin-1-yl, 5H-,6H-,7H-,8H-,8aH-pyrido[3,2-b]pyridin-1-yl, 2H-,7H-,8H-pyrido[3,2-b]pyridin-1-yl, 2H-, 5H-,8H-pyrido[3,2-b]pyridin-1-yl, 2H-,5H-,6H-pyrido[3,2-b]pyridin-1-yl, 2H-,5H-,6H-,7H-, 8H-pyrido[3,2-b]pyridin-1-yl, 4H-,7H-,8H-pyrido[3,2-b]pyridin-1-yl, 4H-,5H-,6H-pyrido[3,2-b]pyridin-1-yl, 4H-pyrido[3,2-b]pyridin-1-yl, 2H-,3H-,7H-,8H-,8 aH-pyrido[3,2-b]pyridin-1-yl, 2H-,3H-,5H-,8H-,8 aH-pyrido[3,2-b]pyridin-1-yl, 2H-,3H-,5H-,6H-,8 aH-pyrido[3,2-b]pyridin-1-yl, 2H-,3H-,5H-,6H-,7H-pyrido[3,2-b]pyridin-1-yl, 2H-,3H-,5H-pyrido[3,2-b]pyridin-1-yl, 2H-,3H-,7H-pyrido[3,2-b]pyridin-1-yl, 2H-,3H-,8 aH-pyrido[3,2-b]pyridin-1-yl, 2H-,6H-,7H-,8H-,8 aH-pyrido[3,2-b]pyridin-1-yl, 2H-,5H-,5aH-,8H-,8 aH-pyrido[3,2-b]pyridin-1-yl, 2H-,5H-,5aH-,6H-pyrido[3,2-b]pyridin-1-yl, 2H-,5H-,5aH-,6H-, 7H-pyrido[3,2-b]pyridin-1-yl, 2H-,5H-,6H-,7H-,8H-pyrido[3,2-b]pyridin-1-yl, 2H-,7H-,8H-pyrido[3,2-b]pyridin-1-yl, 2H-,5H-,6H-pyrido[3,2-b]pyridin-1-yl, 2H-,5H-,8H-pyrido[3,2-b]pyridin-1-yl, 2H-,6H-,7H-pyrido[3,2-b]pyridin-1-yl, 2H-,5aH-,8 aH-pyrido[3,2-b]pyridin-1-yl, 2H-,6H-,8aH-pyrido[3,2-b]pyridin-1-yl, 2H-,5aH-,7H-pyrido[3,2-b]pyridin-1-yl, 4H-,6H-, 7H-,8H-,8aH-pyrido[3,2-b]pyridin-1-yl, 4H-,5 H-,5 aH-,8 H-,8 aH-pyrido[3,2-b]pyridin-1-yl, 4H-,5H-,5 aH-,6H-pyrido[3,2-b]pyridin-1-yl, 4H-,5H-,5 aH-,6H-,7 H-pyrido[3,2-b]pyridin-1-yl, 4H-,5H-,6H-,7H-,8H-pyrido[3,2-b]pyridin-1-yl, 4H-,7H-,8H-pyrido[3,2-b]pyridin-1-yl, 4H-, 5H-,6H-pyrido[3,2-b]pyridin-1-yl, 4H-,5H-,8H-pyrido[3,2-b]pyridin-1-yl, 4H-,6H-,7H-pyrido[3,2-b]pyridin-1-yl, 4H-,5aH-,8 aH-pyrido[3,2-b]pyridin-1-yl, 2H-,3H-,4H-,7H-,8H-pyrido[3,2-b]pyridin-1-yl, 2H-,3H-,4H-,5H-,8H-pyrido[3,2-b]pyridin-1-yl, 2H-,3H-,4H-,5H-, 6H-pyrido[3,2-b]pyridin-1-yl, 2H-,3H-,4H-,6H-,7H-pyrido[3,2-b]pyridin-1-yl, 2H-,3H-,4H-, 5aH-,8 aH-pyrido[3,2-b]pyridin-1-yl, 2H-,3H-,4H-,5H-,8H-pyrido[3,2-b]pyridin-1-yl, 2H-, 3H-,4H-,5aH-,7H-pyrido[3,2-b]pyridin-1-yl, 2H-,3H-,4H-,6H-,8 aH-pyrido[3,2-b]pyridin-1-yl, 2H-,3H-,4H-,5H-,6H-pyrido[3,2-b]pyridin-1-yl, 2H-,3H-,4H-,7H-,8H-pyrido[3,2-b]pyridin-1-yl, 4H-,6H-,8 aH-pyrido[3,2-b]pyridin-1-yl, 4H-,5aH-,7H-pyrido[3,2-b]pyridin-1-yl,

5H-pyrido[4,3-b]pyridin-1-yl, 2H-pyrido[4,3-b]pyridin-1-yl, 4H-,5H-,6H-,7H-,8H-pyrido[4,3-b]pyridin-1-yl, 2H-,5H-,5aH-,6H-,7H-,8H-,8 aH-pyrido[4,3-b]pyridin-1-yl, 2H-, 3H-,5H-,6H-,7H-,8H-,8aH-pyrido[4,3-b]pyridin-1-yl, 4H-,5H-,5Ha-,6H-,7H-,8H-,8aH-pyrido[4,3-b]pyridin-1-yl, 2H-,3H-,4H-,5H-,6H-,7H-,8H-pyrido[4,3-b]pyridin-1-yl, 8 aH-pyrido[4,3-b]pyridin-1-yl, 7H-,8H-,8 aH-pyrido[4,3-b]pyridin-1-yl, 5H-,8H-,8 aH-pyrido[4,3-b]pyridin-1-yl, 5 H-,6H-,8 aH-pyrido[4,3-b]pyridin-1-yl, 5 H-,6H-pyrido[4,3-b]pyridin-1-yl, 5H-,6H-,7H-,8H-,8aH-pyrido[4,3-b]pyridin-1-yl, 2H-,7H-,8H-pyrido[4,3-b]pyridin-1-yl, 2H-, 5H-,8H-pyrido[4,3-b]pyridin-1-yl, 2H-,5H-,6H-pyrido[4,3-b]pyridin-1-yl, 2H-,5H-,6H-,7H-, 8H-pyrido[4,3-b]pyridin-1-yl, 4H-,7H-,8H-pyrido[4,3-b]pyridin-1-yl, 4H-,5H-,6H-pyrido[4,3-b]pyridin-1-yl, 4H-pyrido[4,3-b]pyridin-1-yl, 2H-,3H-,7H-,8H-,8 aH-pyrido[4,3-b]pyridin-1-yl, 2H-,3H-,5H-,8H-,8 aH-pyrido[4,3-b]pyridin-1-yl, 2H-,3H-,5H-,6H-,8 aH-pyrido[4,3-b]pyridin-1-yl, 2H-,3H-,5H-,6H-,7H-pyrido[4,3-b]pyridin-1-yl, 2H-,3H-,5H-pyrido[4,3-b]pyridin-1-yl, 2H-,3H-,7H-pyrido[4,3-b]pyridin-1-yl, 2H-,3H-,8 aH-pyrido[4,3-b]pyridin-1-yl, 2H-,6H-,7H-,8H-,8 aH-pyrido[4,3-b]pyridin-1-yl, 2H-,5H-,5aH-,8H-,8 aH-pyrido[4,3-b]pyridin-1-yl, 2H-,5H-,5aH-,6H-pyrido[4,3-b]pyridin-1-yl, 2H-,5H-,5aH-,6H-, 7H-pyrido[4,3-b]pyridin-1-yl, 2H-,5H-,6H-,7H-,8H-pyrido[4,3-b]pyridin-1-yl, 2H-,7H-,8H-pyrido[4,3-b]pyridin-1-yl, 2H-,5H-,6H-pyrido[4,3-b]pyridin-1-yl, 2H-,5H-,8H-pyrido[4,3-b]pyridin-1-yl, 2H-,6H-,7H-pyrido[4,3-b]pyridin-1-yl, 2H-,5aH-,8 aH-pyrido[4,3-b]pyridin-1-yl, 2H-,6H-,8aH-pyrido[4,3-b]pyridin-1-yl, 2H-,5aH-,7H-pyrido[4,3-b]pyridin-1-yl, 4H-,6H-, 7H-,8H-,8aH-pyrido[4,3-b]pyridin-1-yl, 4H-,5 H-,5aH-,8 H-,8 aH-pyrido[4,3-b]pyridin-1-yl, 4H-,5H-,5aH-,6H-pyrido[4,3-b]pyridin-1-yl, 4H-,5H-,5aH-,6H-,7H-pyrido[4,3-b]pyridin-1-yl, 4H-,5H-,6H-,7H-,8H-pyrido[4,3-b]pyridin-1-yl, 4H-,7H-,8H-pyrido[4,3-b]pyridin-1-yl, 4H-, 5H-,6H-pyrido[4,3-b]pyridin-1-yl, 4H-,5H-,8H-pyrido[4,3-b]pyridin-1-yl, 4H-,6H-,7H-pyrido[4,3-b]pyridin-1-yl, 4H-,5aH-,8 aH-pyrido[4,3-b]pyridin-1-yl, 2H-,3H-,4H-,7H-,8H-pyrido[4,3-b]pyridin-1-yl, 2H-,3H-,4H-,5H-,8H-pyrido[4,3-b]pyridin-1-yl, 2H-,3H-,4H-,5H-, 6H-pyrido[4,3-b]pyridin-1-yl, 2H-,3H-,4H-,6H-,7H-pyrido[4,3-b]pyridin-1-yl, 2H-,3H-,4H-, 5aH-,8 aH-pyrido[4,3-b]pyridin-1-yl, 2H-,3H-,4H-,5H-,8H-pyrido[4,3-b]pyridin-1-yl, 2H-, 3H-,4H-,5aH-,7H-pyrido[4,3-b]pyridin-1-yl, 2H-,3H-,4H-,6H-,8 aH-pyrido[4,3-b]pyridin-1-yl, 2H-,3H-,4H-,5H-,6H-pyrido[4,3-b]pyridin-1-yl, 2H-,3H-,4H-,7H-,8H-pyrido[4,3-b]pyridin-1-yl, 4H-,6H-,8 aH-pyrido[4,3-b]pyridin-1-yl, 4H-,5aH-,7H-pyrido[4,3-b]pyridin-1-yl,

2H-,3H-,4H-,5H-,5aH-,7H-,8H-pyrido[4,3-b]pyridin-6-yl, 2H-,3H-,4H-,7H-,8H-pyrido[4,3-b]pyridin-6-yl, 2H-,3H-,4H-,5H-,5aH-,7H-pyrido[4,3-b]pyridin-6-yl, 2H-,3H-,4H-pyrido[4,3-b]pyridin-6-yl, 3H-,4H-,5H-,5aH-,7H-,8H-,8aH-pyrido[4,3-b]pyridin-6-yl, 3H-, 4H-,7H-,8H-,8aH-pyrido[4,3-b]pyridin-6-yl, 3H-,4H-,5H-,5aH-,7H-,8aH-pyrido[4,3-b]pyridin-6-yl, 3H-,4H-,5H-,5aH-,7H-pyrido[4,3-b]pyridin-6-yl, 3H-,4H-,7H-pyrido[4,3-b]pyridin-6-yl, 3H-,4H-,8aH-pyrido[4,3-b]pyridin-6-yl, 4H-,5H-,5aH-,7H-,8H-pyrido[4,3-b]pyridin-6-yl, 4H-,7H-,8H-pyrido[4,3-b]pyridin-6-yl, 4H-,5H-pyrido[4,3-b]pyridin-6-yl, 4H-pyrido[4,3-b]pyridin-6-yl, 3H-,4H-,5H-,7H-,8H-pyrido[4,3-b]pyridin-6-yl, 3H-,4H-,5H-pyrido[4,3-b]pyridin-6-yl, 2H-,5H-,5aH-,7H-,8H-pyrido[4,3-b]pyridin-6-yl, 2H-,7H-,8H-pyrido[4,3-b]pyridin-6-yl, 2H-,5H-pyrido[4,3-b]pyridin-6-yl, 2H-pyrido[4,3-b]pyridin-6-yl, 3H-,5H-,7H-,8H-,8aH-pyrido[4,3-b]pyridin-6-yl, 3H-,5H-,8aH-pyrido[4,3-b]pyridin-6-yl, 3H-, 5H-pyrido[4,3-b]pyridin-6-yl, 5H-,7H-,8H-pyrido[4,3-b]pyridin-6-yl, 5H-pyrido[4,3-b]pyridin-6-yl, 1H-,4H-,5H-,7H-,8H-pyrido[4,3-b]pyridin-6-yl, 1H-,2H-,3H-,5H-,7H-,8H-, 8aH-pyrido[4,3-b]pyridin-6-yl, 1H-,4H-,5H-,5Ha-,7H-,8H-,8aH-pyrido[4,3-b]pyridin-6-yl, 1H-,2H-,3H-,4H-,5H-,7H-,8H-pyrido[4,3-b]pyridin-6-yl, 1H-,5H-,8aH-pyrido[4,3-b]pyridin-6-yl, 1H-,5H-pyrido[4,3-b]pyridin-6-yl, 1H-,5H-,7H-,8H-,8aH-pyrido[4,3-b]pyridin-6-yl, 1H-, 2H-,5H-pyrido[4,3-b]pyridin-6-yl, 1H-,2H-,5H-,7H-,8H-pyrido[4,3-b]pyridin-6-yl, 1H-,4H-, 5H-pyrido[4,3-b]pyridin-6-yl, 1H-,2H-,3H-,5H-,8aH-pyrido[4,3-b]pyridin-6-yl, 1H-,2H-,3H-, 5H-,7H-pyrido[4,3-b]pyridin-6-yl, 1H-,2H-,7H-,8H-,8aH-pyrido[4,3-b]pyridin-6-yl, 1H-,2H-, 5H-,5aH-pyrido[4,3-b]pyridin-6-yl, 1H-,2H-,5H-,5aH-,7H-pyrido[4,3-b]pyridin-6-yl, 1H-, 2H-,5H-,7H-,8H-pyrido[4,3-b]pyridin-6-yl, 1H-,2H-,5H-pyrido[4,3-b]pyridin-6-yl, 1H-,2H-, 5H-,8H-pyrido[4,3-b]pyridin-6-yl, 1H-,2H-,7H-pyrido[4,3-b]pyridin-6-yl, 1H-,2H-,8aH-pyrido[4,3-b]pyridin-6-yl, 1H-,4H-,7H-,8H-,8aH-pyrido[4,3-b]pyridin-6-yl, 1H-,4H-,5H-, 5aH-pyrido[4,3-b]pyridin-6-yl, 1H-,4H-,5H-,5aH-,7H-pyrido[4,3-b]pyridin-6-yl, 1H-,4H-, 5H-,7H-,8H-pyrido[4,3-b]pyridin-6-yl, 1H-,4H-,5H-pyrido[4,3-b]pyridin-6-yl, 1H-,4H-,7H-pyrido[4,3-b]pyridin-6-yl, 1H-,2H-,3H-,4H-,5H-pyrido[4,3-b]pyridin-6-yl, 1H-,2H-,3H-,4H-, 7H-pyrido[4,3-b]pyridin-6-yl, 1H-,2H-,3H-,4H-,8aH-pyrido[4,3-b]pyridin-6-yl, 1H-,2H-,3H-, 4H-,5H-pyrido[4,3-b]pyridin-6-yl, 1H-,4H-,8aH-pyrido[4,3-b]pyridin-6-yl,

2H-,4H-(1,3-benzoxazin-3-yl), 2H-,4H-,5H-,8H-(1,3-benzoxazin-3-yl), 2H-,4H-,7H-, 8H-(1,3-benzoxazin-3-yl), 2H-,4H-,5H-,6H-(1,3-benzoxazin-3-yl), 2H-,4H-,5H-,6H-,7H-, 8H-(1,3-benzoxazin-3-yl), 2H-,8aH-(1,3-benzoxazin-3-yl), 2H-,5H-(1,3-benzoxazin-3-yl), 2H-,5H-,6H-,7H-(1,3-benzoxazin-3-yl), 2H-,5H-,8H-,8aH-(1,3-benzoxazin-3-yl), 2H-,7H-, 8H-,8aH-(1,3-benzoxazin-3-yl), 2H-,7H-,8H-,8aH-(1,3-benzoxazin-3-yl), 2H-,7H-(1,3-benzoxazin-3-yl), 2H-,5H-,6H-,7H-,8H-,8aH-(1,3-benzoxazin-3-yl), 2H-,4H-,5aH-,8aH-(1,3-benzoxazin-3-yl), 2H-,4H-,5aH-,7H-(1,3-benzoxazin-3-yl), 2H-,4H-,6H-(1,3-benzoxazin-3-yl), 2H-,4H-,6H-,7H-,8H-,8aH-(1,3-benzoxazin-3-yl), 2H-,4H-,5aH-,7H-,8H-,8aH-(1,3-benzoxazin-3-yl), 2H-,4H-,5H-,5aH-,8H-,8aH-1,3-benzoxazin-3-yl), 2H-,4H-,5H-,5aH-,6H-, 8aH-(1,3-benzoxazin-3-yl), 2H-,4H-,5H-,5aH-,6H-,7H-(1,3-benzoxazin-3-yl), 2H-,4H-,5H-, 5aH-,6H-,7H-,8H-,8aH-(1,3-benzoxazin-3-yl),

5H-,5aH-,6H-,7H-,8H-,8aH-(1,4-benzoxazin-4-yl), 6H-,7H-,8H-,8aH-(1,4-benzoxazin-4-yl), 5aH-,7H-,8H-,8aH-(1,4-benzoxazin-4-yl), 5H-,5aH-,8H-,8aH-(1,4-benzoxazin-4-yl), 5H-,5aH-,6H-,8aH-(1,4-benzoxazin-4-yl), 5H-,5aH-,6H-,7H-(1,4-benzoxazin-4-yl), 5aH-,7H-(1,4-benzoxazin-4-yl), 6H-,8aH-(1,4-benzoxazin-4-yl), 8H-,8aH-(1,4-benzoxazin-4-yl), 5H-,5aH-(1,4-benzoxazin-4-yl), 2H-,3H-,5H-,6H-,7H-,8H-(1,4-benzoxazin-4-yl), 2H-,3H-,7H-,8H-(1,4-benzoxazin-4-yl), 2H-,3H-,5H-,8H-(1,4-benzoxazin-4-yl), 2H-,3H-,5H-,6H-(1,4-benzoxazin-4-yl), 5H-,6H-,7H-,8H-(1,4-benzoxazin-4-yl), 7H-, 8H-(1,4-benzoxazin-4-yl), 5H-,8H-(1,4-benzoxazin-4-yl), 5H-,6H-(1,4-benzoxazin-4-yl), 1,4-benzoxazin-4-yl, 2H-,3H-,5H-,5aH-,6H-,7H-,8H-,8aH-(1,4-benzoxazin-4-yl), 2H-,3H-, 6H-,7H-,8H-,8aH-(1,4-benzoxazin-4-yl), 2H-,3H-,5aH-,7H-,8H-,8aH-(1,4-benzoxazin-4-yl), 2H-,3H-,5H-,5aH-,8H-,8aH-(1,4-benzoxazin-4-yl), 2H-,3H-,5H-,5aH-,6H-,8H-(1,4-benzoxazin-4-yl), 2H-,3H-,5H-,5aH-,6H-,7H-(1,4-benzoxazin-4-yl), 2H-,3H-,6H-,7H-(1,4-benzoxazin-4-yl), 2H-,3H-,5aH-,7H-(1,4-benzoxazin-4-yl), 2H-,3H-,5H-,5aH-(1,4-benzoxazin-4-yl), 2H-,3H-,6H-,8aH-(1,4-benzoxazin-4-yl), 2H-,3H-,8H-,8aH-(1,4-benzoxazin-4-yl),

5H-,5aH-,6H-,7H-,8H-,8aH-(2,3-benzoxazin-3-yl), 5aH-,7H-,8H-(2,3-benzoxazin-3-yl), 5H-,8H-(2,3-benzoxazin-3-yl), 5H-,6H-(2,3-benzoxazin-3-yl), 2,3-benzoxazin-3-yl, 1H-, 4H-,5H-,6H-,7H-,8H-(2,3-benzoxazin-3-yl), 1H-,4H-,7H-,8H-(2,3-benzoxazin-3-yl), 1H-, 4H-,5H-,8H-(2,3-benzoxazin-3-yl), 1H-,4H-,5H-,6H-(2,3-benzoxazin-3-yl), 1H-,4H-(2,3-benzoxazin-3-yl), 4H-,6H-,7H-,8H-(2,3-benzoxazin-3-yl), 4H-,7H-,8H-(2,3-benzoxazin-3-yl), 4H-,5H-,8H-(2,3-benzoxazin-3-yl), 4H-,5H-,6H-(2,3-benzoxazin-3-yl), 4H-,5H-,5aH-,6H-, 7H-,8H-,8aH-(2,3-benzoxazin-3-yl), 4H-,8H-(2,3-benzoxazin-3-yl), 4H-,6H-(2,3-benzoxazin-3-yl), 4H-,5aH-(2,3-benzoxazin-3-yl), 1H-,5H-,6H-,7H-,8H-,8aH-(2,3-benzoxazin-3-yl), 1H-,7H-,8H-,8aH-(2,3-benzoxazin-3-yl), 1H-,5H-,7H-,8H-,8aH-(2,3-benzoxazin-3-yl), 1H-,5H-,6H-,8aH-(2,3-benzoxazin-3-yl), 1H-,5H-,6H-,7H-(2,3-benzoxazin-3-yl), 1H-,8aH-(2,3-benzoxazin-3-yl), 1H-,7H-(2,3-benzoxazin-3-yl), 1H-,5H-(2,3-benzoxazin-3-yl), 1H-,4H-,5H-,5aH-,6H-,7H-,8H-,8aH-(2,3-benzoxazin-3-yl), 1H-,4H-, 6H-,7H-,8H-,8aH-(2,3-benzoxazin-3-yl), 1H-,4H-,5aH-,7H-,8H-,8aH-(2,3-benzoxazin-3-yl), 1H-,4H-,5H-,5aH-,8H-,8aH-(2,3-benzoxazin-3-yl), 1H-,4H-,5H-,5aH-,6H-,8aH-(2,3-benzoxazin-3-yl), 1H-,4H-,5H-,5aH-,6H-,7H-(2,3-benzoxazin-3-yl), 1H-,4H-,6H-,7H-(2,3-benzoxazin-3-yl), 1H-,4H-,5aH-,7H-(2,3-benzoxazin-3-yl), 1H-,4H-,5H-,5aH-(2,3-benzoxazin-3-yl), 1H-,4H-,6H-,8aH-(2,3-benzoxazin-3-yl), 1H-,4H-,8H-,8aH-(2,3-benzoxazin-3-yl),

3,1-benzoxazin-1-yl, 2H-,5H-,6H-,7H-,8H-,8aH-(3,1-benzoxazin-1-yl), 2H-,4H-,5H-, 6H-,7H-,8H-(3,1-benzoxazin-1-yl), 2H-,7H-,8H-,8aH-(3,1-benzoxazin-1-yl), 2H-,5H-,8H-, 8aH-(3,1-benzoxazin-1-yl), 2H-,5H-,6H-,8aH-(3,1-benzoxazin-1-yl), 2H-,5H-,6H-,7H-(3,1-benzoxazin-1-yl), 2H-,5H-(3,1-benzoxazin-1-yl), 2H-,7H-(3,1-benzoxazin-1-yl), 2H-,8 aH-(3,1-benzoxazin-1-yl), 2H-,4H-,7H-,8H-(3,1-benzoxazin-1-yl), 2H-,4H-,5H-,8H-(3,1-benzoxazin-1-yl), 2H-,4H-,5H-,6H-(3,1-benzoxazin-1-yl), 2H-,4H-,6H-,7H-(3,1-benzoxazin-1-yl), 2H-,4H-,5 aH-,8aH-(3,1-benz oxazin-1-yl), 2H-,4H-,5H-,8H-(3,1-benzoxazin-1-yl), 2H-, 4H-,5 aH-,7H-(3,1-benzoxazin-1-yl), 2H-,4H-,6H-,8aH-(3,1-benzoxazin-1-yl), 2H-,4H-,5H-, 6H-(3,1-benzoxazin-1-yl), 2H-,4H-,7H-,8H-(3,1-benzoxazin-1-yl),

1,2-benzisoxazin-2-yl, 5H-,6H-(1,2-benzisoxazin-2-yl), 5H-,8H-(1,2-benzisoxazin-2-yl), 7H-,8H-(1,2-benzisoxazin-2-yl), 5H-,6H-,7H-,8H-(1,2-benzisoxazin-2-yl),

3H-,8aH-(1,2-benzisoxazin-2-yl), 3H-,7H-(1,2-benzisoxazin-2-yl), 3H-,5H-(1,2-benzisoxazin-2-yl), 3H-,7H-,8H-,8aH-(1,2-benzisoxazin-2-yl), 3H-,5H-,8H-,8aH-(1,2-benzisoxazin-2-yl), 3H-,5H-,6H-,8aH-(1,2-benzisoxazin-2-yl), 3H-,5H-,6H-,7H-(1,2-benzisoxazin-2-yl), 3H-,5H-,6H-,7H-,8H-,8aH-(1,2-benzisoxazin-2-yl),

3H-,4H-,7H-,8H-(1,2-benzisoxazin-2-yl), 3H-,4H-,7H-,8H-(1,2-benzisoxazin-2-yl), 3H-,4H-,5H-,8H-(1,2-benzisoxazin-2-yl), 3H-,4H-,5H-,6H-(1,2-benzisoxazin-2-yl), 3H-,4H-, 5H-,6H-,7H-,8H-(1,2-benzisoxazin-2-yl),

5aH-,8aH-(1,2-benzisoxazin-2-yl), 5aH-,7H-(1,2-benzisoxazin-2-yl), 5aH-,7H-,8H-, 8aH-(1,2-benzisoxazin-2-yl), 5H-,5aH-,8H-,8aH-(1,2-benzisoxazin-2-yl), 5H-,5aH-,6H-, 8aH-(1,2-benzisoxazin-2-yl), 5H-,5aH-,6H-,7H-(1,2-benzisoxazin-2-yl), 6H-,8aH-(1,2-benzisoxazin-2-yl), 8H-,8aH-(1,2-benzisoxazin-2-yl), 6H-,7H-(1,2-benzisoxazin-2-yl), 5H-, 5aH-,6H-,7H-,8H-,8aH-(1,2-benzisoxazin-2-yl),

3H-,4H-,5H-,5aH-,6H-,7H-,8H-,8aH-(1,2-benzisoxazin-2-yl), 3H-,4H-,6H-,7H-,8H-, 8aH-(1,2-benzisoxazin-2-yl), 3H-,4H-,5aH-,7H-,8H-,8aH-(1,2-benzisoxazin-2-yl), 3H-,4H-, 5H-,5aH-,8H-,8aH-(1,2-benzisoxazin-2-yl), 3H-,4H-,5H-,5aH-,6H-,8aH-(1,2-benzisoxazin-2-yl), 3H-,4H-,5H-,5aH-,6H-,7H-(1,2-benzisoxazin-2-yl), 3H-,4H-,5H-,6H-,7H-,8H-(1,2-benzisoxazin-2-yl), 3H-,4H-,7H-,8H-(1,2-benzisoxazin-2-yl), 3H-,4H-,5H-,8H-(1,2-benzisoxazin-2-yl), 3H-,4H-,6H-,7H-(1,2-benzisoxazin-2-yl), 3H-,4H-,5H-,6H-(1,2-benzisoxazin-2-yl), 3H-,4H-,5aH-,8aH-(1,2-benzisoxazin-2-yl), 3H-,4H-,8H-,8aH-(1,2-benzisoxazin-2-yl), 3H-,4H-,6H-,8aH-(1,2-benzisoxazin-2-yl), 3H-,4H-,5aH-,7H-(1,2-benzisoxazin-2-yl), 3H-,4H-,5H-,5aH-(1,2-benzisoxazin-2-yl),

purin-1-yl, purin-3-yl, purin-7-yl, purin-9-yl, or hydro compound thereof,

carbazol-9-yl, or hydro compound thereof,

and salt thereof.

The heterocyclic ring group can be unsubstituted or substituted with one or more than one substituent. If substituted, the substituent group takes the place of one or more hydrogens in the heterocyclic ring group. Combinations of substituents are possible. Exemplary substituents include hydroxyl, halo, bromo, chloro, iodo, fluoro, —OR¹², —NR¹²R¹³, NR¹²COR¹³, —CONR¹²R¹³, CONHR¹², —COOR¹², —OCOR¹², —COR¹², —SR¹², SO₂R¹², —SO₃R¹², —SO₂NHR¹², —SOR¹², —N₃, —CN, —NC, —SH, —NO₂, —NH₂, PR₂, —(O)PR¹²R¹³, —PO₃R¹²R¹³, OPO₃R¹²R¹³, —PO₂, (C₁-C₂₀) alkyl, phenyl, (C₃-C₂₀) cycloalkyl, (C₁-C₂₀) alkoxy, (C₃-C₂₅) heteroaryl, (C₃-C₂₅) heterocyclyl, (C₂-C₂₀) alkenyl, (C₄-C₂₀) cycloalkenyl, (C₂-C₂₀) alkynyl, (C₆-C₂₀) cycloalkynyl, (C₅-C₂₅) aryl, perhalo (C₁-C₂₀) alkyl, salt thereof, or a combination thereof.

In one embodiment, each of R¹, R², R³, R⁴, R⁵, R⁶, R⁷, R⁸, R⁹, R¹⁰, and R¹¹ may be independently a (C₃-C₂₅) heterocyclyl, as a pendant group, selected from the group including furan, thiophene, pyrrole, isopyrrole, pyrazole, isodiazole, triazole, dithole, oxathole, isoxazole, thiazole, isothiazole, oxadiazole, dioxazole, oxathiole, oxathiazole, pyran, pyridine, pyridazine, pyrimidine, pyrazine, piperazine, morpholino, triazine, oxazine, isoxazine, oxathiazine, azepine, oxepin, thiepin, diazepine, benzofuran, isobenzofuran, thionaphthene, isothionaphthene, indole, isbenzazole, pyrindine, pyrano[3,4-b]pyrrole, isoindazole, indoxazine, benzoxazole, anthranil, benzopyran, quinoline, isoquinoline, cinnoline, quinazoline, nahthyridine, pyrido[3,4-b]pyridine, pyrido[3,2-b]pyridine, pyrido[4,3-b-]pyridine, benzoxazine, carbazole, xanthene, acridine, purine, hydro compound thereof, and salt thereof. The heterocyclyl can be substituted or unsubstituted with one or more substituents. Combinations of substituents are possible.

The (C₁-C₂₀) alkyl may be a C₁, C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, or C₂₀ alkyl. It may be substituted or unsubstituted. It may be branched or unbranched.

The phenyl includes a C₆ phenyl. It may be substituted or unsubstituted.

The (C₃-C₂₀) cycloalkyl may be a C₃, C₄, C₅, C₆, C₇, C₈, C₉, C₁₀, C₁₁, C₁₂, C₁₃, C₁₄, C₁₅, C₁₆, C₁₇, C₁₈, C₁₉, or C₂₀ cycloalkyl. It may be substituted or unsubstituted. It may be branched or unbranched.

The (C₁-C₂₀) alkoxy may be a C₁, C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, or C₂₀ alkoxy. It may be substituted or unsubstituted. It may be branched or unbranched.

The (C₃-C₂₅) heteroaryl may be a C₃, C₄, C₅, C₆, C₇, C₈, C₉, C₁₀, C₁₁, C₁₂, C₁₃, C₁₄, C₁₅, C₁₆, C₁₇, C₁₈, C₁₉, C₂₀, C₂₁, C₂₂, C₂₃, C₂₄, or C₂₅ heteroaryl, wherein one or more of the carbons are replaced with an N, O, or S atom. More than one carbon may be replaced. Combinations of N, O, and S are possible. It may be substituted or unsubstituted. It may be branched or unbranched.

The (C₃-C₂₅) heterocyclyl may be a C₃, C₄, C₅, C₆, C₇, C₈, C₉, C₁₀, C₁₁, C₁₂, C₁₃, C₁₄, C₁₅, C₁₆, C₁₇, C₁₈, C₁₉, C₂₀, C₂₁, C₂₂, C₂₃, C₂₄, C₂₅ heterocyclyl, wherein one or more of the carbons are replaced with an N, O, or S atom. More than one carbon may be replaced. Combinations of N, O, and S are possible. It may be substituted or unsubstituted. It may be branched or unbranched.

The (C₂-C₂₀) alkenyl may be a C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, or C₂₀ alkenyl. It may be substituted or unsubstituted. It may be branched or unbranched.

The (C₄-C₂₀) cycloalkenyl may be a C₄, C₅, C₆, C₇, C₈, C₉, C₁₀, C₁₁, C₁₂, C₁₃, C₁₄, C₁₅, C₁₆, C₁₇, C₁₈, C₁₉, or C₂₀ cycloalkenyl. It may be substituted or unsubstituted. It may be branched or unbranched.

The (C₂-C₂₀) alkynyl may be a C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, or C₂₀ alkynyl. It may be substituted or unsubstituted. It may be branched or unbranched.

The (C₆-C₂₀) cycloalkynyl may be a C₆, C₇, C₈, C₉, C₁₀, C₁₁, C₁₂, C₁₃, C₁₄, C₁₅, C₁₆, C₁₇, C₁₈, C₁₉, or C₂₀ cycloalkynyl. It may be substituted or unsubstituted. It may be branched or unbranched.

The (C₅-C₂₅) aryl may be a C₅, C₆, C₇, C₈, C₉, C₁₀, C₁₁, C₁₂, C₁₃, C₁₄, C₁₅, C₁₆, C₁₇, C₁₈, C₁₉, C₂₀, C₂₁, C₂₂, C₂₃, C₂₄, or C₂₅ aryl. It may be substituted or unsubstituted. It may be branched or unbranched.

The perhalo (C₁-C₂₀) alkyl may be a C₁, C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, or C₂₀ alkyl in which all or some of the hydrogens are replaced by halogens, F, Cl, Br, or I. Combinations of halogens are possible. It may be substituted or unsubstituted. It may be branched or unbranched.

The (C₃-C₆) cycloalkyl structure may be a C₃, C₄, C₅, or C₆ cycloalkyl structure, formed from R³ and R⁴ groups taken together on adjacent Xs. The cycloalkyl structure may be substituted or unsubstituted. It may be branched or unbranched.

The (C₅-C₆) aryl structure may be a C₅ or C₆ aryl structure, formed from two R groups taken together, for example, R⁵ and R⁶, on adjacent atoms. The cycloalkyl structure may be substituted or unsubstituted. It may be branched or unbranched.

The (C₃-C₆) heteroaryl structure may be a C₃, C₄, C₅, or C₆ heteroaryl structure, formed from two R groups taken together, for example, R⁵ and R⁶, on adjacent atoms, wherein one or more of the carbons are replaced with an N, O, or S atom. More than one carbon may be replaced. Combinations of N, O, and S are possible. The cycloalkyl structure may be substituted or unsubstituted. It may be branched or unbranched.

The (C₃-C₆) heterocyclyl structure may be a C₃, C₄, C₅, or C₆ heterocyclyl structure, formed from two R groups taken together, for example, R⁵ and R⁶, on adjacent atoms, wherein one or more of the carbons are replaced with an N, O, or S atom and having one or more double bonds. More than one carbon may be replaced. Combinations of N, O, and S are possible. The heterocyclyl structure may be substituted or unsubstituted. It may be branched or unbranched.

The (C₃-C₆) heterocycloalkenyl structure may be a C₃, C₄, C₅, or C₆ heterocycloalkenyl structure, formed from two R groups taken together, for example, R⁵ and R⁶, on adjacent atoms, wherein one or more of the carbons are replaced with an N, O, or S atom and having one or more double bonds. More than one carbon may be replaced. Combinations of N, O, and S are possible. The heterocycloalkenyl structure may be substituted or unsubstituted. It may be branched or unbranched.

The (C₄-C₆) cycloalkenyl structure may be a C₄, C₅, or C₆ cycloalkenyl structure, formed from two R groups taken together, for example, R⁵ and R⁶, on adjacent atoms and having one or more double bonds. The cycloalkenyl structure may be substituted or unsubstituted. It may be branched or unbranched.

One embodiment includes a composition, in which a compound having formula (I), salt thereof, or a combination thereof is present, together with at least one pharmaceutically acceptable carrier.

One embodiment includes a composition, in which more than one compound of formula (I), salt thereof, or a combination thereof is present. For example, the composition may include different compounds falling within formula (I) and/or salt thereof. One embodiment includes a composition having different salts of the same compound of formula (I).

One embodiment includes a composition, in which at least one compound having formula (I), salt, or a combination thereof is present, together with at least one known inhibitor, such as Nucleoside analog Reverse Transcriptase inhibitor (NRTi), —Non-Nucleoside analog Reverse Transcriptase inhibitor (NNRTi), Protease inhibitor (Pi), or Cell Entry inhibitor (Ci). Combinations are possible.

Some examples of the Nucleoside analog Reverse Transcriptase inhibitor (NRTi) include AZT, 3TC, FTC, ABC, ddC, ZDV, TDF, ddI, DFC, DAPD, alovidine, elvucitabine, D4T, RCV, or Beta-L-Fd4C. Combinations are possible.

Examples of the Non-Nucleoside analog Reverse Transcriptase inhibitor (NNRTi) include DLV, EFV, —NVP, calanolide A, etravirine, TMC-278, BMS-561390, or capravirine. Combinations are possible.

Examples of the Protease inhibitor (Pi) include APV, TPV, IDV, SQV, LPV, FPV, RTV, ATZ, —NFV, brecanavir, darunavir, PPL-100, L-756423, or RO033-4649. Combinations are possible.

Examples of the Cell Entry inhibitor (Ci) include Fuzeon, ENF, aplaviroc, maraviroc, vicriviroc, T-1249, PRO-542, TNX-355, SCH-C, PRO-140, SP-01A, SP-10, or TNX-355. Combinations are possible.

The inhibitors may be administered with the compound separately or in the same composition. If administered separately, the inhibitor may be administered in advance of, concurrently with, or after the administration of the compound.

Given the compounds and the teachings herein, combined with the knowledge of one of ordinary skill in organic and/or synthetic chemistry, one can synthesize the compounds herein without undue effort or experimentation.

The compound can be in the form of a salt. Examples of salts include acid or base addition salts. Exemplary acids which may be used to prepare the acid addition salts of the compounds include those which form non-toxic acid addition salts, e.g., salts containing pharmacologically acceptable anions, such as the chloride, bromide, iodide, nitrate, sulfate, bisulfate, phosphate, acid phosphate, citrate, acid citrate, tartrate, bitartrate, succinate, fumarate, tosylate, mesylate, gluconate, saccharate, benzoate, methanesulfonate, ethanesulfonate, benzenesulfonate, p-toluenesulfonate and pamoate (i.e., 1,1′-methylene-bis-(2-hydroxy-3-naphthoate)) salts.

Other salts include, for example, benzensulfonate, benzoate, bicarbonate, bitartrate, edetate, camsylate, carbonate, citrate, dihydrochloride, edentate, edisylate, estolate, esylate, fumarate, gluceptate, glucoheptonate, gluconate, glutamate, glycollylarsanilate, hexylresorcinate, hydrabamine, hydrobromide, hydrochloride, hydroxynaphthoate, chloride, bromide, iodide, isethionate, lactate, lactobionate, malate, mandelate, mesylate, methylbromide, methylnitrate, methylsulfate, mucate, napsylate, nitrate, pamoate, pantothenate, phosphate or diphosphate, polygalacturonate, salicylate, stearate, subacetate, succinate, sulfate, tannate, teoclate, and triethiodide salts.

Other suitable salts include sodium, potassium, ammonium, calcium, or ferric hydroxide salts, isopropylamine, trimethylamine, 2-ethylamino ethanol, histidine, procaine, hydrochloric or phosphoric acids, organic acids such as acetic, oxalic, tartaric, mandelic, and the like.

Any ratio of compound:counterion in the salt form is suitable, for example, compound:counterion ratios of 10, 9, 8, 7, 6, 5, 4, 3, 2, 1:1, 2, 3, 4, 5, 6, 7, 8, 9, 10 is suitable. The ratio can be expressed as the number of compounds:counterions or as the number of ionic moieties in the compound:counterions as appropriate. In one embodiment, either the compound or the counterion or both may be multivalent, and the ratio is adjusted accordingly such that the salt may adopt a zero or non-zero charge. Mixed salts are possible.

The compound may be administered to the subject in any form or mode which makes the compound bioavailable in effective amounts, or improves its bioavailability, including orally, subcutaneously, intramuscularly, intravenously, transdermally, intranasally, rectally, topically, intramucosaly, intravaginally, parenterally, and the like. Given the teachings herein, one skilled in the art can readily select the proper form and mode of administration depending upon the compound selected, the malady to be treated, the stage of the disease, and other relevant circumstances.

The compound may be administered alone or in the form of a pharmaceutical composition in combination with one or more pharmaceutically acceptable carriers so long as the composition is suitable for administration to a mammalian subject, and particularly a human. In one embodiment, one or more prodrugs of the compound are contemplated for administration. Mixtures are possible.

The compound or composition may be suitably administered batchwise or by constant or periodic infusion over an extended period of time, for example, exceeding 24 hours, until the desired therapeutic, preventive, and/or inhibiting benefits are obtained.

The carrier is physiologically tolerable by a human and does not interfere with the intended effect of the active compound. Some nonlimiting examples of the pharmaceutically acceptable carrier include water, physiological saline, ethanol, aqueous ethanol, dimethyl sulfoxide, castor oil, benzyl alcohol, benzyl benzoate, albumin, polyethylene glycol, cellulose, fatty acid, methylcellulose, dextrose, glycerol, mannitol, lactose, starch, magnesium stearate, sodium saccharin, or magnesium carbonate, or a combination thereof.

The suitability of particular carriers for inclusion in a given composition may depend on the route of administration desired. For example, the composition may be prepared as a liquid solution, suspension, emulsion, cream, inhalant, patch, implant, solid, tablet, pill, capsule, sustained release, or powder form. The composition may include such one or more additives or excipients such as binder, filler, preservative, stabilizing agent, emulsifier, wetting agent, emulsifying agent, stabilizing agent, pH buffering agent, and the like. Combinations are possible.

The composition may contain 1% to 95% by weight of active compound, as appropriate, be it the compound, salt, tautomer, prodrug, or a combination thereof. This range includes all values and subranges therebetween, including 1, 2, 3, 4, 5, 6, 7, 8, 9, 10, 20, 30, 40, 50, 60, 70, 80, 90, and 95% by weight, and any combination thereof.

The term “therapeutically effective amount” refers to an amount of the compound, prodrug, salt, or combination thereof which is effective, upon single or multiple dose administration or continuous administration, infusion or application to the patient, for the treatment of the malady. A therapeutically effective amount can be readily determined by the attending diagnostician, as one skilled in the art, by the use of known techniques and by observing results obtained under analogous circumstances. In determining the therapeutically effective amount or dose, a number of factors are considered by the attending diagnostician, including, but not limited to the subjects size, age, sex, and general health; the malady involved; the degree of or involvement or the severity of the disease; the response of the individual subject; the particular compound administered; the bioavailability characteristics of the preparation administered; the dose regimen selected; the use of concomitant medication; and other relevant circumstances.

A therapeutically effective amount of the compound may range from about 0.0001 milligram per kilogram of body weight per day (mg/kg/day) to about 10,000 mg/kg/day. Preferred amounts may range from about 0.001 to about 100 mg/kg/day. These ranges include all values and subranges therebetween, including 0.0001, 0.0005, 0.001, 0.005, 0.01, 0.05, 0.1, 0.5, 1, 2, 3, 4, 5, 6, 7, 8, 9, 10, 100, 1,000, 5,000, and 10,000 mg/kg/day, and any combination thereof.

What is claimed is:

1. A compound having the formula: 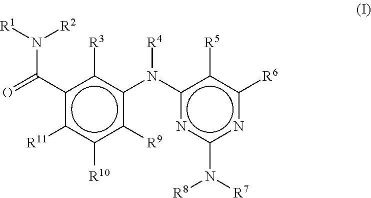
or salt thereof;
wherein R¹ and R² are each independently selected from the group consisting of hydrogen, hydroxyl, halo, bromo, chloro, iodo, fluoro, —NR¹²COR¹³, —CONR¹²R¹³, —CONHR¹², —COOR¹², —OCOR¹², —COR¹², —SR¹², —SO₂R¹², —SO₂R¹², —SO₂NHR¹², —SOR¹², —CN, —NC, —SH, —NO₂, —NH₂, —PR₂, —(O)PR¹²R¹³, —PO₃R¹²R¹³, —OPO₃R¹²R¹³, —PO₂, (C₁-C₂₀) alkyl, phenyl, (C₃-C₂₀) cycloalkyl, (C₁-C₂₀) alkoxy, (C₃-C₂₅) heteroaryl, (C₃-C₂₅) heterocyclyl, (C₂-C₂₀) alkenyl, (C₄-C₂₀) cycloalkenyl, (C₂-C₂₀) alkynyl, (C₆-C₂₀) cycloalkynyl, (C₅-C₂₅) aryl, perhalo (C₁-C₂₀) alkyl, and salt thereof;
or wherein R¹ and R² together with the nitrogen to which they are attached form a heterocyclic ring group or salt thereof;
wherein R³, R⁹, and R¹¹ are each independently selected from the group consisting of hydrogen, hydroxyl, halo, bromo, chloro, iodo, fluoro, halo, bromo, chloro, iodo, fluoro, —OR¹², —NR¹²R¹³, —NR¹²COR¹³, —CONR¹²R¹³, —CONHR¹², —COOR¹², —OCOR¹², —COR¹², —SR¹², —SO₂R¹², —SO₃R¹², —SO₂NHR¹², —SOR¹², —N₃, —CN, —NC, —SH, —NO₂, —NH₂, —PR₂, —(O)PR¹²R¹³, —PO₃R¹²R¹³, OPO₃R¹²R¹³, —PO₂, (C₁-C₂₀) alkyl, phenyl, (C₃-C₂₀) cycloalkyl, (C₁-C₂₀) alkoxy, (C₃-C₂₅) heteroaryl, (C₃-C₂₅) heterocyclyl, (C₂-C₂₀) alkenyl, (C₄-C₂₀) cycloalkenyl, (C₂-C₂₀) alkynyl, (C₆-C₂₀) cycloalkynyl, (C₅-C₂₅) aryl, perhalo (C₁-C₂₀) alkyl, and salt thereof;
wherein R⁴ is hydrogen, hydroxyl, halo, bromo, chloro, iodo, fluoro, —OR¹², —NR¹²R¹³, —NR¹²COR¹³, —CONR¹²R¹³, —CONHR¹², —COOR¹², —OCOR¹², —COR¹², —SR¹², —SO₂R¹², —SO₃R¹², —SO₂NHR¹², —SOR¹², —CN, —NC, —SH, —NO₂, —NH₂, PR₂,—(O)PR¹²R¹³, —PO₃R¹²R¹³, —OPO₃R¹²R¹³, —PO₂, (C₁-C₂₀) alkyl, phenyl, (C₃-C₂₀) cycloalkyl, (C₁-C₂₀) alkoxy, (C₃-C₂₅) heteroaryl, (C₃-C₂₅) heterocyclyl, (C₂-C₂₀) alkenyl, (C₄-C₂₀) cycloalkenyl, (C₂-C₂₀) alkynyl, (C₆-C₂₀) cycloalkynyl, (C₅-C₂₅) aryl, perhalo (C₁-C₂₀) alkyl, or salt thereof
wherein R⁵ and R⁶ are each independently selected from the group consisting of hydrogen, hydroxyl, halo, bromo, chloro, iodo, fluoro, —OR¹², —NR¹²R¹³, —NR¹²COR¹³, CONR¹²R¹³, —CONHR¹², —COOR¹², —OCOR¹², —COR¹², —SR¹², —SO₂R¹², —SO₃R¹², —SO₂NHR¹², —SOR¹², —N₃, —CN, —NC, —SH, —NO₂, —NH₂, —PR₂, —(O)PR¹²R¹³, —PO₃R¹²R¹³, —OPO₃R¹²R¹³, —PO₂, (C₁-C₂₀) alkyl, phenyl, (C₃-C₂₀) cycloalkyl, (C₁-C₂₀) alkoxy, (C₃-C₂₅) heteroaryl, (C₃-C₂₅) heterocyclyl, (C₂-C₂₀) alkenyl, (C₄-C₂₀) cycloalkenyl, (C₂-C₂₀) alkynyl, (C₆-C₂₀) cycloalkynyl, (C₅-C₂₅) aryl, perhalo (C₁-C₂₀) alkyl, and salt thereof;
or wherein R⁵ and R⁶ together with the carbons to which they are attached form a (C₅-C₆) aryl structure, a (C₃-C₆) heteroaryl structure, a (C₃-C₆) heterocycloalkenyl structure, a (C₄-C₆) cycloalkenyl structure, or salt thereof;
wherein R⁷ and R⁸ are each independently selected from the group consisting of hydrogen, hydroxyl, halo, bromo, chloro, iodo, fluoro, —C(NH)NH₂, —C(NH)NHC(NH)NH₂, —OR¹², —NR¹²R¹³, —NR¹²COR¹³, —CONR¹²R¹³, —CONHR¹², —COOR¹², —OCOR¹², —COR¹², —SR¹², —SO₂R¹², —SO₃R¹², —SO₂NHR¹², —SOR¹², CN, —NC, —SH, —NO₂, —NH₂, —PR₂, —(O)PR¹²R¹³, —PO₃R¹²R¹³, —OPO₃R¹²R¹³, PO₂, (C₁-C₂₀) alkyl, phenyl, (C₃-C₂₀) cycloalkyl, (C₁-C₂₀) alkoxy, (C₃-C₂₅) heteroaryl, (C₃-C₂₅) heterocyclyl, (C₂-C₂₀) alkenyl, (C₄-C₂₀) cycloalkenyl, (C₂-C₂₀) alkynyl, (C₆-C₂₀) cycloalkynyl, (C₅-C₂₅) aryl, perhalo (C₁-C₂₀) alkyl, and salt thereof;
or wherein R⁷ and R⁸ together with the nitrogen to which they are attached form a heterocyclic ring group or salt thereof;
wherein R¹⁰ is hydrogen, hydroxyl, halo, bromo, chloro, iodo, fluoro, —OR¹⁴, —NR¹⁴R¹⁵, —NR¹⁴COR¹⁵, —CONR¹⁴R¹⁵, —CONR¹⁴, —COOR¹⁴, —OCOR¹⁴, —COR¹⁴, SR¹⁴, —SO₂R¹⁴, —SO₃R¹⁴, —SO₂NR¹⁴, —SOR¹⁴, —N₃, —CN, —NC, —SH, —NO₂, NH₂, —PR₂, —(O)PR¹⁴R¹⁵, —PO₃R¹⁴R¹⁵, —OPO₃R¹⁴R¹⁵, —PO₂, (C₁-C₂₀) alkyl, phenyl, (C₃-C₂₀) cycloalkyl, (C₁-C₂₀) alkoxy, (C₃-C₂₅) heteroaryl, (C₃-C₂₅) heterocyclyl, (C₂-C₂₀) alkenyl, (C₄-C₂₀) cycloalkenyl, (C₂-C₂₀) alkynyl, (C₆-C₂₀) cycloalkynyl, (C₅-C₂₅) aryl, perhalo (C₁-C₂₀) alkyl, or salt thereof;
wherein R¹² and R¹³ are each independently selected from the group consisting of (C₁-C₂₀) alkyl, phenyl, (C₃-C₂₀) cycloalkyl, (C₁-C₂₀) alkoxy, (C₃-C₂₅) heteroaryl, (C₃-C₂₅) heterocyclyl, (C₂-C₂₀) alkenyl, (C₄-C₂₀) cycloalkenyl, (C₂-C₂₀) alkynyl, (C₆-C₂₀) cycloalkynyl, (C₅-C₂₅) aryl, perhalo (C₁-C₂₀) alkyl, and salt thereof;
wherein R¹⁴ and R¹⁵ are each independently selected from the group consisting of hydrogen, hydroxyl, halo, bromo, chloro, iodo, fluoro, —OR¹², —NR¹²R¹³, —NR¹²COR¹³, CONR¹²R¹³, —CONHR¹², —COOR¹², —OCOR¹², —COR¹², —SR¹², —SO₂R¹², —SO₃R¹², —SO₂NHR¹², —SOR¹², —N₃, —CN, —NC, —SH, —NO₂, —NH₂, —PR₂, —(O)PR¹²R¹³, —PO₃R¹²R¹³, —OPO₃R¹²R¹³, —PO₂, (C₁-C₂₀) alkyl, phenyl, (C₃-C₂₀) cycloalkyl, (C₁-C₂₀) alkoxy, (C₃-C₂₅) heteroaryl, (C₃-C₂₅) heterocyclyl, (C₂-C₂₀) alkenyl, (C₄-C₂₀) cycloalkenyl, (C₂-C₂₀) alkynyl, (C₆-C₂₀) cycloalkynyl, (C₅-C₂₅) aryl, perhalo (C₁-C₂₀) alkyl, and salt thereof;
and wherein each of said (C₁-C₂₀) alkyl, phenyl, (C₃-C₂₀) cycloalkyl, (C₁-C₂₀) alkoxy, (C₃-C₂₅) heteroaryl, (C₃-C₂₅) heterocyclyl, (C₂-C₂₀) alkenyl, (C₄-C₂₀) cycloalkenyl, (C₂-C₂₀) alkynyl, (C₆-C₂₀) cycloalkynyl, (C₅-C₂₅) aryl, perhalo (C₁-C₂₀) alkyl, (C₃-C₆) cycloalkyl structure, (C₅-C₆) aryl structure, (C₃-C₆) heteroaryl structure, (C₃-C₆) heterocyclyl structure, (C₃-C₆) heterocycloalkenyl structure, (C₄-C₆) cycloalkenyl structure, heterocyclic ring group, or salt thereof may be independently unsubstituted or substituted with one or more substituents selected from the group consisting of hydroxyl, halo, bromo, chloro, iodo, fluoro, —OR¹², —NR¹²R¹³, —NR¹²COR¹³, —CONR¹²R¹³, —CONHR¹², —COOR¹², —OCOR¹², —COR¹², —SR¹², —SO₂R¹², —SO₃R¹², —SO₂NHR¹², —SOR¹², —N₃, —CN, —NC, —SH, NO₂, —NH₂, —PR₂, —(O)PR¹²R¹³, —PO₃R¹²R¹³, —OPO₃R¹²R¹³, —PO₂, (C₁-C₂₀) alkyl, phenyl, (C₃-C₂₀) cycloalkyl, (C₁-C₂₀) alkoxy, (C₃-C₂₅) heteroaryl, (C₃-C₂₅) heterocyclyl, (C₂-C₂₀) alkenyl, (C₄-C₂₀) cycloalkenyl, (C₂-C₂₀) alkynyl, (C₆-C₂₀) cycloalkynyl, (C₅-C₂₅) aryl, perhalo (C₁-C₂₀) alkyl, salt thereof, and a combination thereof;
with the proviso that when R³, R¹⁰, and R¹¹ are hydrogen, and R⁹ is —CH₃, then R⁵ is not —CN.

2. The compound of claim 1, wherein R¹ and R² are each independently selected from the group consisting of hydrogen, hydroxyl, halo, bromo, chloro, iodo, and fluoro.

3. The compound of claim 1, wherein R¹ and R² are each independently selected from the group consisting of —NR¹²COR¹³, —CONR¹²R¹³, —CONHR¹², —COOR¹², —OCOR¹², —COR¹², —SR¹², —SO₂R¹², —SO₃R¹², —SO₂NHR¹², —SOR¹², —N₃, —CN, —NC, —SH, —NO₂, —NH₂, —PR₂, —(O)PR¹²R¹³, —PO₃R¹²R¹³, OPO₃R¹²R¹³, —PO₂, and salt thereof.

4. The compound of claim 1, wherein R¹ and R² are each independently selected from the group consisting of (C₁-C₂₀) alkyl, phenyl, (C₃-C₂₀) cycloalkyl, (C₁-C₂₀) alkoxy, (C₃-C₂₅) heteroaryl, (C₃-C₂₅) heterocyclyl, (C₂-C₂₀) alkenyl, (C₄-C₂₀) cycloalkenyl, (C₂-C₂₀) alkynyl, (C₆-C₂₀) cycloalkynyl, (C₅-C₂₅) aryl, perhalo (C₁-C₂₀) alkyl, and salt thereof.

5. The compound of claim 1, wherein R¹ and R² together with the nitrogen to which they are attached form a heterocyclic ring group or salt thereof.

6. The compound of claim 1, wherein R³, R⁹, and R¹¹ are each independently selected from the group consisting of hydrogen, hydroxyl, halo, bromo, chloro, iodo, fluoro.

7. The compound of claim 1, wherein R³, R⁹, and R¹¹ are each independently selected from the group consisting of —OR¹², —NR¹²COR¹³, —CONR¹²R¹³, —CONHR¹², —COOR¹², —OCOR¹², —COR¹², —SR¹², —SO₂R¹², —SO₃R¹², —SO₂NHR¹², —SOR¹², —N₃, —CN, —NC, —SH, —NO₂, —NH₂, —PR₂, —(O)PR¹²R¹³, —PO₃R¹²R¹³, —OPO₃R¹²R¹³, —PO₂, and salt thereof.

8. The compound of claim 1, wherein R³, R⁹, and R¹¹ are each independently selected from the group consisting of (C₁-C₂₀) alkyl, phenyl, (C₃-C₂₀) cycloalkyl, (C₁-C₂₀) alkoxy, (C₃-C₂₅) heteroaryl, (C₃-C₂₅) heterocyclyl, (C₂-C₂₀) alkenyl, (C₄-C₂₀) cycloalkenyl, (C₂-C₂₀) alkynyl, (C₆-C₂₀) cycloalkynyl, (C₅-C₂₅) aryl, perhalo (C₁-C₂₀) alkyl, and salt thereof.

9. The compound of claim 1, wherein R⁴ is selected from the group consisting of hydrogen, hydroxyl, halo, bromo, chloro, iodo, and fluoro.

10. The compound of claim 1, wherein R⁴ is selected from the group consisting of —OR¹², —NR¹²R¹³, —NR¹²COR¹³, —CONR¹²R¹³, —CONHR¹², —COOR¹², —OCOR¹², —COR¹², —SR¹², —SO₂R¹², —SO₃R¹², —SO₂NHR¹², —SOR¹², —CN, —NC, —SH, —NO₂, —NH₂, —PR₂, —(O)PR¹²R¹³, —PO₃R¹²R¹³, —PO₃R¹²R¹³, —PO₂, and salt thereof.

11. The compound of claim 1, wherein R⁴ is selected from the group consisting of (C₁-C₂₀) alkyl, phenyl, (C₃-C₂₀) cycloalkyl, (C₁-C₂₀) alkoxy, (C₃-C₂₅) heteroaryl, (C₃-C₂₅) heterocyclyl, (C₂-C₂₀) alkenyl, (C₄-C₂₀) cycloalkenyl, (C₂-C₂₀) alkynyl, (C₆-C₂₀) cycloalkynyl, (C₅-C₂₅) aryl, perhalo (C₁-C₂₀) alkyl, and salt thereof.

12. The compound of claim 1, wherein R⁵ and R⁶ are each independently selected from the group consisting of hydrogen, hydroxyl, halo, bromo, chloro, iodo, and fluoro.

13. The compound of claim 1, wherein R⁵ and R⁶ are each independently selected from the group consisting of —OR¹², —NR¹²COR¹³, —CONR¹²R¹³, —CONHR¹², —COOR¹², —OCOR¹², —COR¹², —SR¹², —SO₂R¹², —SO₃R¹², —SO₂NHR¹², —SOR¹², —N₃, —CN, —NC, —SH, —NO₂, —NH₂, —PR₂, —(O)PR¹²R¹³, —PO₃R¹²R¹³, —OPO₃R¹²R¹³, —PO₂, and salt thereof.

14. The compound of claim 1, wherein R⁵ and R⁶ are each independently selected from the group consisting of (C₁-C₂₀) alkyl, phenyl, (C₃-C₂₀) cycloalkyl, (C₁-C₂₀) alkoxy, (C₃-C₂₅) heteroaryl, (C₃-C₂₅) heterocyclyl, (C₂-C₂₀) alkenyl, (C₄-C₂₀) cycloalkenyl, (C₂-C₂₀) alkynyl, (C₆-C₂₀) cycloalkynyl, (C₅-C₂₅) aryl, perhalo (C₁-C₂₀) alkyl, and salt thereof.

15. The compound of claim 1, wherein R⁵ and R⁶ together with the carbons to which they are attached form a (C₅-C₆) aryl structure, a (C₃-C₆) heteroaryl structure, a (C₃-C₆) heterocycloalkenyl structure, a (C₄-C₆) cycloalkenyl structure, or salt thereof.

16. The compound of claim 1, wherein R⁷ and R⁸ are each independently selected from the group consisting of hydrogen, hydroxyl, halo, bromo, chloro, iodo, and fluoro.

17. The compound of claim 1, wherein R⁷ and R⁸ are each independently selected from the group consisting of —C(NH)NH₂, —C(NH)NHC(NH)NH₂, —OR¹², —NR¹²R¹³, —NR¹²COR¹³, —CONR¹²R¹³, —CONHR¹², —COOR¹², —OCOR¹², —COR¹², —SR¹², —SO₂R¹², —SO₃R¹², —SO₂NHR¹², —SOR¹², —CN, —NC, —SH, —NO₂, —NH₂, —PR₂, —(O)PR¹²R¹³, —PO₃R¹²R¹³, —OPO₃R¹²R¹³, —PO₂, and salt thereof.

18. The compound of claim 1, wherein R⁷ and R⁸ are each independently selected from the group consisting of (C₁-C₂₀) alkyl, phenyl, (C₃-C₂₀) cycloalkyl, (C₁-C₂₀) alkoxy, (C₃-C₂₅) heteroaryl, (C₃-C₂₅) heterocyclyl, (C₂-C₂₀) alkenyl, (C₄-C₂₀) cycloalkenyl, (C₂-C₂₀) alkynyl, (C₆-C₂₀) cycloalkynyl, (C₅-C₂₅) aryl, perhalo (C₁-C₂₀) alkyl, and salt thereof.

19. The compound of claim 1, wherein R⁷ and R⁸ together with the nitrogen to which they are attached form a heterocyclic ring group or salt thereof.

20. The compound of claim 1, wherein R¹⁰ is selected from the group consisting of hydrogen, hydroxyl, halo, bromo, chloro, iodo, and fluoro.

21. The compound of claim 1, wherein R¹⁰ is selected from the group consisting of —OR¹⁴, —NR¹⁴R¹⁵, —NR¹⁴COR¹⁵, —CONR¹⁴R¹⁵, —CONR¹⁴, —COOR¹⁴, —OCOR¹⁴, —COR¹⁴, —SR¹⁴, —SO₂R¹⁴, —SO₃R¹⁴, —SO₂NR¹⁴, —SOR¹⁴, —N₃, —CN, —NC, —SH, —NO₂, —NH₂, —PR₂, —(O)PR¹⁴R¹⁵, —PO₃R¹⁴R¹⁵, —OPO₃R¹⁴R¹⁵, —PO₂, and salt thereof.

22. The compound of claim 1, wherein R¹⁰ is selected from the group consisting of (C₁-C₂₀) alkyl, phenyl, (C₃-C₂₀) cycloalkyl, (C₁-C₂₀) alkoxy, (C₃-C₂₅) heteroaryl, (C₃-C₂₅) heterocyclyl, (C₂-C₂₀) alkenyl, (C₄-C₂₀) cycloalkenyl, (C₂-C₂₀) alkynyl, (C₆-C₂₀) cycloalkynyl, (C₅-C₂₅) aryl, perhalo (C₁-C₂₀) alkyl, and salt thereof.

23. The compound of claim 1, wherein R¹⁴ and R¹⁵ are each independently selected from the group consisting of hydrogen, hydroxyl, halo, bromo, chloro, iodo, and fluoro.

24. The compound of claim 1, wherein R¹⁴ and R¹⁵ are each independently selected from the group consisting of —OR¹², —NR¹²R¹³, —NR¹²COR¹³, —CONR¹²R¹³, —CONHR¹², —COOR¹², —OCOR¹², —COR¹², —SR¹², —SO₂R¹², —SO₃R¹², —SO₂NHR¹², —SOR¹², —N₃, —CN, —NC, —SH, —NO₂, —NH₂, —PR₂, —(O)PR¹²R¹³, —PO₃R¹²R¹³, —OPO₃R¹²R¹³, —PO₂, and salt thereof.

25. The compound of claim 1, wherein R¹⁴ and R¹⁵ are each independently selected from the group consisting of (C₁-C₂₀) alkyl, phenyl, (C₃-C₂₀) cycloalkyl, (C₁-C₂₀) alkoxy, (C₃-C₂₅) heteroaryl, (C₃-C₂₅) heterocyclyl, (C₂-C₂₀) alkenyl, (C₄-C₂₀) cycloalkenyl, (C₂-C₂₀) alkynyl, (C₆-C₂₀) cycloalkynyl, (C₅-C₂₅) aryl, perhalo (C₁-C₂₀) alkyl, and salt thereof.

26. The compound of claim 1, wherein at least one (C₁-C₂₀) alkyl, phenyl, (C₃-C₂₀) cycloalkyl, (C₁-C₂₀) alkoxy, (C₃-C₂₅) heteroaryl, (C₃-C₂₅) heterocyclyl, (C₂-C₂₀) alkenyl, (C₄-C₂₀) cycloalkenyl, (C₂-C₂₀) alkynyl, (C₆-C₂₀) cycloalkynyl, (C₅-C₂₅) aryl, perhalo (C₁-C₂₀) alkyl, (C₃-C₆) cycloalkyl structure, (C₅-C₆) aryl structure, (C₃-C₆) heteroaryl structure, (C₃-C₆) heterocyclyl structure, (C₃-C₆) heterocycloalkenyl structure, (C₄-C₆) cycloalkenyl structure, heterocyclic ring group, or salt thereof is substituted with one or more substituents selected from the group consisting of hydroxyl, halo, bromo, chloro, iodo, fluoro, —OR¹², —NR¹²R¹³, —NR¹²COR¹³, —CONR¹²R¹³, —CONHR¹², —COOR¹², —OCOR¹², —COR¹², —SR¹², —SO₂R¹², —SO₃R¹², —SO₂NHR¹², —SOR¹², —N₃, —CN, —NC, —SH, —NO₂, —NH₂, —PR₂, —(O)PR¹²R¹³, —PO₃R¹²R¹³, —OPO₃R¹²R¹³, —PO₂, (C₁-C₂₀) alkyl, phenyl, (C₃-C₂₀) cycloalkyl, (C₁-C₂₀) alkoxy, (C₃-C₂₅) heteroaryl, (C₃-C₂₅) heterocyclyl, (C₂-C₂₀) alkenyl, (C₄-C₂₀) cycloalkenyl, (C₂-C₂₀) alkynyl, (C₆-C₂₀) cycloalkynyl, (C₅-C₂₅) aryl, perhalo (C₁-C₂₀) alkyl, salt thereof, and a combination thereof.

27. The compound of claim 1, wherein at least one (C₁-C₂₀) alkyl, phenyl, (C₃-C₂₀) cycloalkyl, (C₁-C₂₀) alkoxy, (C₃-C₂₅) heteroaryl, (C₃-C₂₅) heterocyclyl, (C₂-C₂₀) alkenyl, (C₄-C₂₀) cycloalkenyl, (C₂-C₂₀) alkynyl, (C₆-C₂₀) cycloalkynyl, (C₅-C₂₅) aryl, perhalo (C₁-C₂₀) alkyl, (C₃-C₆) cycloalkyl structure, (C₅-C₆) aryl structure, (C₃-C₆) heteroaryl structure, (C₃-C₆) heterocyclyl structure, (C₃-C₆) heterocycloalkenyl structure, (C₄-C₆) cycloalkenyl structure, heterocyclic ring group, or salt thereof is not substituted.

28. The compound of claim 1, wherein at least one of R³, R¹⁰, or R¹¹ is not hydrogen.

29. The compound of claim 1, wherein R⁹ is not —CH₃.

30. The compound of claim 1, wherein at least one of R¹ and R² together with the nitrogen to which they are attached, R⁷ and R⁸ together with the nitrogen to which they are attached, or both independently forms a heterocyclic ring group selected from the group consisting of:
pyrrolyl, 2,3-dihydropyrrol-1-yl, 2,5-dihydropyrrol-1-yl, tetrahydropyrrol-1-yl,
pyrazol-1-yl, 3H-pyrazol-1-yl, 4H-pyrazol-1-yl, 3,4-dihydropyrazol-1-yl,
1,3-isodiazol-1-yl, 3H-(1,3-isodiazol-1-yl), 3,4-dihydro-(1,3-isodiazol-1-yl),
1,2,3-triazol-1-yl, 2H-(1,2,3-triazol-1-yl), 2,3-dihydro(1,2,3-triazol-1-yl), 2,3,4-trihydro(1,2,3-triazol-1-yl),
1,2,3-triazol-2-yl, 1,3-dihydro(1,2,3-triazol-2-yl), 1,3,4-trihydro(1,2,3-triazol-2-yl),
1,2,4-triazol-1-yl, 2H-(1,2,4-triazol-1-yl), 2,3-dihydro(1,2,4-triazol-1-yl), 2,4-dihydro(1,2,4-triazol-1-yl),
1,2,4-triazol-4-yl, 1H-(1,2,4-triazol-4-yl), 1,2-dihydro(1,2,4-triazol-4-yl), 3,5-dihydro(1,2,4-triazol-4-yl),
isoxazol-2-yl, 3H-isoxazol-2-yl, 4H-isoxazol-2-yl,
oxazol-3-yl, 4H-oxazol-3-yl,
thiazol-3-yl, 4H-thiazol-3-yl,
isothiazol-2-yl, 3H-isothiazol-2-yl,
1,2,3-oxadiazol-2-yl, 3H-(1,2,3-oxadiazol-2-yl),
1,2,3-oxadiazol-3-yl, 4H-(1,2,3-oxadiazol-3-yl),
1,2,4-oxidiazol-2-yl, 3H-(1,2,4-oxidiazol-2-yl), 4H-(1,2,4-oxidiazol-2-yl), 5H-(1,2,4-oxidiazol-2-yl),
1,2,4-oxidiazol-4-yl, 3H-(1,2,4-oxidiazol-4-yl),
1,2,5-oxadiazol-2-yl, 4H-(1,2,5-oxadiazol-2-yl),
1,3,4-oxadiazol-3-yl, 4H-(1,3,4-oxadiazol-3-yl),
1,2,3,4-oxatriazol-2-yl, 3H-(1,2,3,4-oxatriazol-2-yl), 5H-(1,2,3,4-oxatriazol-2-yl), 3H-, 4H-(1,2,3,4-oxatriazol-2-yl),
1,2,3,4-oxatriazol-3-yl, 4H-(1,2,3,4-oxatriazol-3-yl),
5H-(1,2,3,5-oxatriazol-2-yl), 3H-,4H-(1,2,3,5-oxatriazol-2-yl),
1,2,3,5-oxatriazol-3-yl, 4H-(1,2,3,5-oxatriazol-3-yl),
1,2,3,5-oxatriazol-5-yl, 4H-(1,2,3,5-oxatriazol-5-yl), 2H-(1,2,3,5-oxatriazol-5-yl), 2H-, 3H-(1,2,3,5-oxatriazol-5-yl),
1,2,3-dioxazol-3-yl, 4H-(1,2,3-dioxazol-3-yl),
1,2,4-dioxazol-4-yl,
1,3,2-dioxazol-2-yl, 4H-(1,3,2-dioxazol-2-yl),
1,3,4-dioxazol-4-yl,
1,2,5-oxathiazol-5-yl, 3H-(1,2,5-oxathiazol-5-yl),
2H-(pyridin-1-yl), 4H-(pyridin-1-yl), 3H-,4H-(pyridin-1-yl), 2H-,5H-(pyridin-1-yl), 3H-,5H-(pyridin-1-yl),
2H-(pyridazin-1-yl), 4H-(pyridazin-1-yl), 6H-(pyridazin-1-yl), 3H-,4H-(pyridazin-1-yl), 4H-,5H-(pyridazin-1-yl), 2H-,5H-(pyridazin-1-yl), 2H-,3H-,6H-(pyridazin-1-yl), 3H-,5H-(pyridazin-1-yl),
2H-(pyrimidin-1-yl), 4H-(pyrimidin-1-yl), 6H-(pyrimidin-1-yl), 3H-,4H-(pyrimidin-1-yl), 4H-,5H-(pyrimidin-1-yl), 2H-,5H-(pyrimidin-1-yl), 2H-,3H-,6H-(pyrimidin-1-yl), 3H-, 5H-(pyrimidin-1-yl),
2H-(pyrazin-1-yl), 4H-(pyrazin-1-yl), 3H-,4H-(pyrazin-1-yl), 2H-,5H-(pyrazin-1-yl), piperazin-1-yl,
2H-(s-triazin-1-yl), 4H-(s-triazin-1-yl), 4H-,5H-(s-triazin-1-yl), 2H-,5H-(s-triazin-1-yl), 3H-,5H-(s-triazin-1-yl),
2H-(as-triazin-1-yl), 4H-(as-triazin-1-yl), 6H-(as-triazin-1-yl), 3H-,4H-(as-triazin-1-yl), 4H-,5H-(as-triazin-1-yl), 2H-,5H-(as-triazin-1-yl), 2H-,3H-,6H-(as-triazin-1-yl), 3H-,5H-(as-triazin-1-yl),
1H-(as-triazin-2-yl), 3H-(as-triazin-2-yl), 5H-,6H-(as-triazin-2-yl), 3H-,6H-(as-triazin-2-yl), 1H-,4H-(as-triazin-2-yl), 5H-(as-triazin-2-yl), 1H-,3H-,5H-(as-triazin-2-yl), 3H-,5H-(as-triazin-2-yl),
1H-(as-triazin-4-yl), 3H-(as-triazin-4-yl), 5H-(as-triazin-4-yl), 3H-,6H-(as-triazin-4-yl), 2H-,5H-(as-triazin-4-yl), 1H-,5H-(as-triazin-4-yl), 1H-,3H-(as-triazin-4-yl), 1H-,3H-,5H-(as-triazin-4-yl),
2H-(v-triazin-1-yl), 4H-(v-triazin-1-yl), 6H-(v-triazin-1-yl), 3H-,4H-(v-triazin-1-yl), 4H-,5H-(v-triazin-1-yl), 2H-,5H-(v-triazin-1-yl), 2H-,3H-,6H-(v-triazin-1-yl), 3H-,5H-(v-triazin-1-yl),
1H-(v-triazin-2-yl), 3H-(v-triazin-2-yl), 3H-,5H-(v-triazin-2-yl), 3H-,6H-(v-triazin-2-yl), 5H-,6H-(v-triazin-2-yl), 5H-(v-triazin-2-yl), 1H-,3H-,5H-(v-triazin-2-yl),
1,2-oxazin-2-yl, 5H-,6H-(1,2-oxazin-2-yl), 3H-,6H-(1,2-oxazin-2-yl), 4H-(1,2-oxazin-2-yl), 3H-,5H-(1,2-oxazin-2-yl),
1,2-oxazin-3-yl, 1H-,4H-(1,2-oxazin-2-yl), 2H-,5H-(1,2-oxazin-2-yl),
1,4-oxazin-4-yl, 5H-(1,4-oxazin-4-yl), morpholin-4-yl,
1,2,4-oxathiazin-4-yl, 5H-(1,2,5-oxathiazin-4-yl),
1,2,5-oxathiazin-5-yl, 3H-(1,2,5-oxathiazin-5-yl),
1,2,6-oxathiazin-6-yl, 3H-(1,2,6-oxathiazin-6-yl), 4H-(1,2,6-oxathiazin-6-yl), 5H-(1,2,6-oxathiazin-6-yl),
1,2,4-oxadiazin-4-yl, 2H-(1,2,4-oxadiazin-4-yl), 5H-(1,2,4-oxadiazin-4-yl), 2H-,5H-(1,2,4-oxadiazin-4-yl),
1,3,5-oxadiazin-3-yl, 4H-(1,3,5-oxadiazin-3-yl),
azepin-1-yl, 2H-,7H-azepin-1-yl, 4H-,6H-azepin-1-yl, 3H-,6H-azepin-1-yl, 4H-,7H-azepin-1-yl, 2H-,4H-,6H-azepin-1-yl,
1,2-diazepin-1-yl, 2H-,7H-(1,2-diazepin-1-yl), 4H-,6H-(1,2-diazepin-1-yl), 3H-,6H-(1,2-diazepin-1-yl), 4H-,7H-(1,2-diazepin-1-yl), 2H-,4H-,6H-(1,2-diazepin-1-yl), 2H-,4H-, 7H-(1,2-diazepin-1-yl), 2H-,4H-,5H-(1,2-diazepin-1-yl), 2H-,5H-(1,2-diazepin-1-yl), 2H-, 3H-(1,2-diazepin-1-yl),
indol-1-yl, 6H-,7H-indol-1-yl, 4H-,7H-indol-1-yl, 4H-,5H-indol-1-yl, 7H-,7 aH-indol-1-yl, 5H-,7aH-indol-1-yl, 5H-,6H-indol-1-yl, 4aH-,7aH-indol-1-yl, 3aH-,6H-indol-1-yl, 3aH-, 4H-indol-1-yl, 4H-,5H-,6H-,7H-indol-1-yl, 5H-,6H-,7H-,7 aH-indol-1-yl, 6H-,7H-,3aH-,7 aH-indol-1-yl, 4H-,7H-,3aH-,7 aH-indol-1-yl, 4H-,5H-,3 aH-,7 aH-indol-1-yl, 4H-,5H-,6H-,3 aH-indol-1-yl,
2H-indol-1-yl, 2H-,4aH-,7aH-indol-1-yl, 2H-,3aH-,6H-indol-1-yl, 2H-,3 aH-,4H-indol-1-yl, 2H-,6H-,7H-,3aH-,7aH-indol-1-yl, 2H-,4H-,7H-,3aH-,7aH-indol-1-yl, 2H-,4H-,5H-, 3aH-,7aH-indol-1-yl, 2H-,4H-,5 H-,6H-,3 aH-indol-1-yl,
2H-,3H-indol-1-yl, 2H-,3H-,6H-,7H-indol-1-yl, 2H-,3H-,4H-,7H-indol-1-yl, 2H-,3H-, 4H-,5H-indol-1-yl, 2H-,3H-,7H-,7 aH-indol-1-yl, 2H-,3H-,5H-,7 aH-indol-1-yl, 2H-,3H-,5H-, 6H-indol-1-yl, 2H-,3H-,4aH—,7 aH-indol-1-yl, 2H-,3H-,3aH-,6H-indol-1-yl, 2H-,3H-,3 aH-, 4H-indol-1-yl, 2H-,3 H-,4H-,5 H-,6H-,7 H-indol-1-yl, 2H-,3 H-,5 H-,6H-,7 H-,7 aH-indol-1-yl, 2H-,3H-,6H-,7H-,3aH-,7aH-indol-1-yl, 2H-,3H-,4H-,7H-,3aH-,7aH-indol-1-yl, 2H-,3H-,4H-, 5H-,3aH-,7 aH-indol-1-yl, 2H-,3H-,4H-,5H-,6H-,3 aH-indol-1-yl,
2-isobenzazol-2-yl, 4H-,5H-(2-isobenzazol-2-yl), 4H-,7H-(2-isobenzazol-2-yl), 1H-, 3H-(2-isobenzazol-2-yl), 1H-,3H-,4H-,5H-(2-isobenzazol-2-yl), 1H-,3H-,4H-,7H-(2-isobenzazol-2-yl), 1H-,3H-,4H-,4aH-,7aH-,7H-(2-isobenzazol-2-yl), 1H-,3H-,4aH-,7aH-(2-isobenzazol-2-yl), 1H-,3H-,5H-,6H-(2-isobenzazol-2-yl), 3H-,4aH-(2-isobenzazol-2-yl), 3H-, 5H-(2-isobenzazol-2-yl), 3H-,7H-(2-isobenzazol-2-yl), 3H-,4aH-,4H-,7aH-,7H-(2-isobenzazol-2-yl), 3H-,4aH-,6H-,7H-(2-isobenzazol-2-yl), 3H-,5H-,6H-,7H-(2-isobenzazol-2-yl), 4H-,4aH-,5H-(2-isobenzazol-2-yl), 1H-,3H-,4aH-,6H-,7H-,7aH-(2-isobenzazol-2-yl), 1H-, 3H-,5H-,6H-,7H-,7aH-(2-isobenzazol-2-yl), 3H-,4H-,4aH-,5H-,6H-,7H-(2-isobenzazol-2-yl), 1H-,3H-,4H-,5H-,6H-,7H-(2-isobenzazol-2-yl), 4H-,5H-,6H-,7H-(2-isobenzazol-2-yl),
1-pyrindin-1-yl, 4H-,5 aH-(1-pyrindin-1-yl), 2H-,5aH-(1-pyrindin-1-yl), 2H-,3H-,4H-, 5aH-(1-pyrindin-1-yl), 2H-,7H-,7 aH-(1-pyrindin-1-yl), 4H-,6H-,7H-,7aH-(1-pyrindin-1-yl), 2H-,3H-,4H-,6H-,7H-,7 aH-(1-pyrindin-1-yl), 2H-,7H-(1-pyrindin-1-yl), 4H-,7H-(1-pyrindin-1-yl), 2H-,3H-,4H-,7H-(1-pyrindin-1-yl), 4H-,5H-,5aH-,7H-,7aH-(1-pyrindin-1-yl), 2H-,3H-, 4H-,5aH-,7H-,7aH-(1-pyrindin-1-yl), 2H-,3H-,4H-,5H-,6H-,7H-,7 aH-(1-pyrindin-1-yl), 2H-, 3H-,4H-,5H-,5aH-,6H-,7H-,7aH-(1-pyrindin-1-yl), 2H-,5H-(1-pyrindin-1-yl), 4H-,5H-(1-pyrindin-1-yl), 2H-,3H-,4H-,5H-(1-pyrindin-1-yl), 2H-,5H-,5aH-,7aH-(1-pyrindin-1-yl), 2H-, 3H-,4H-,5H-,5aH-,7aH-(1-pyrindin-1-yl), 5H-,6H-(1-pyrindin-1-yl), 4H-,5H-,5aH-,6H-(1-pyrindin-1-yl), 2H-,3H-,5H-,6H-(1-pyrindin-1-yl), 2H-,3H-,4H-,5H-,5aH-(1-pyrindin-1-yl), 2H-,6H-(1-pyrindin-1-yl), 4H-,6H-(1-pyrindin-1-yl), 2H-,3H-,4H-,6H-(1-pyrindin-1-yl),
7H-(pyrano[3,4-b]pyrol-1-yl), 5H-(pyrano[3,4-b]pyrol-1-yl), 3aH-(pyrano[3,4-b]pyrol-1-yl), 4H-,5H-,7H-(pyrano[3,4-b]pyrol-1-yl), 5H-,7H-,7aH-(pyrano[3,4-b]pyrol-1-yl), 7H-,3aH-,7aH-(pyrano[3,4-b]pyrol-1-yl), 4H-,5H-,3aH-(pyrano[3,4-b]pyrol-1-yl), 2H-,3aH-(pyrano[3,4-b]pyrol-1-yl), 2H-,7H-,3aH-,7aH-(pyrano[3,4-b]pyrol-1-yl), 2H-,4H-,5H-,3aH-(pyrano[3,4-b]pyrol-1-yl), 2H-,3H-,7H-(pyrano[3,4-b]pyrol-1-yl), 2H-,3H-,5H-(pyrano[3,4-b]pyrol-1-yl), 2H-,3H-,3aH-(pyrano[3,4-b]pyrol-1-yl), 2H-,3H-,4H-,5H-,7H-(pyrano[3,4-b]pyrol-1-yl), 2H-,3H-,5H-,7H-,7aH-(pyrano[3,4-b]pyrol-1-yl), 2H-,3H-,7H-,3aH-,7aH-(pyrano[3,4-b]pyrol-1-yl), 2H-,3H-,4H-,5H-,3aH-(pyrano[3,4-b]pyrol-1-yl),
isoindazol-1-yl, 6H-,7H-isoindazol-1-yl, 4H-,7H-isoindazol-1-yl, 4H-,5H-isoindazol-1-yl, 7H-,7aH-isoindazol-1-yl, 5H-,7aH-isoindazol-1-yl, 5H-,6H-isoindazol-1-yl, 4aH-,7aH-isoindazol-1-yl, 3aH-,6H-isoindazol-1-yl, 3aH-,4H-isoindazol-1-yl, 4H-,5H-,6H-,7H-isoindazol-1-yl, 5H-,6H-,7H-,7aH-isoindazol-1-yl, 6H-,7H-,3aH-,7aH-isoindazol-1-yl, 4H-, 7H-,3aH-,7aH-isoindazol-1-yl, 4H-,5H-,3aH-,7aH-isoindazol-1-yl, 4H-,5H-,6H-,3aH-isoindazol-1-yl,
2H-isoindazol-1-yl, 2H-,4aH-,7 aH-isoindazol-1-yl, 2H-,3aH-,6H-isoindazol-1-yl, 2H-, 3aH-,4H-isoindazol-1-yl, 2H-,6H-,7H-,3aH-,7 aH-isoindazol-1-yl, 2H-,4H-,7H-,3aH-,7 aH-isoindazol-1-yl, 2H-,4H-,5H-,3aH-,7 aH-isoindazol-1-yl, 2H-,4H-,5H-,6H-,3aH-isoindazol-1-yl,
2H-,3H-isoindazol-1-yl, 2H-,3H-,6H-,7H-isoindazol-1-yl, 2H-,3H-,4H-,7H-isoindazol-1-yl, 2H-,3H-,4H-,5H-isoindazol-1-yl, 2H-,3H-,7H-,7aH-isoindazol-1-yl, 2H-,3H-, 5H-,7aH-isoindazol-1-yl, 2H-,3H-,5H-,6H-isoindazol-1-yl, 2H-,3H-,4aH-,7aH-isoindazol-1-yl, 2H-,3H-,3aH-,6H-isoindazol-1-yl, 2H-,3H-,3aH-,4H-isoindazol-1-yl, 2H-,3H-,4H-,5H-, 6H-,7H-isoindazol-1-yl, 2H-,3H-,5H-,6H-,7H-,7aH-isoindazol-1-yl, 2H-,3H-,6H-,7H-,3aH-, 7aH-isoindazol-1-yl, 2H-,3H-,4H-,7H-,3aH-,7 aH-isoindazol-1-yl, 2H-,3H-,4H-,5H-,3aH-, 7 aH-isoindazol-1-yl, 2H-,3H-,4H-,5H-,6H-,3aH-isoindazol-1-yl,
isoindazol-2-yl, 4H-,5H-isoindazol-2-yl, 4H-,7H-isoindazol-2-yl, 1H-,3H-isoindazol-2-yl, 1H-,3H-isoindazol-2-yl, 1H-,3H-,4H-,5H-isoindazol-2-yl, 1H-,3H-,4H-,7H-isoindazol-2-yl, 1H-,3H-,4H-,4aH-,7aH-,7H-isoindazol-2-yl, 1H-,3H-,4aH-,7aH-isoindazol-2-yl, 1H-, 3H-,5H-,6H-isoindazol-2-yl, 3H-,4aH-isoindazol-2-yl, 3H-,5H-isoindazol-2-yl, 3H-,7H-isoindazol-2-yl, 3H-,4aH-,4H-,7aH-,7H-isoindazol-2-yl, 3H-,4aH-,6H-,7H-isoindazol-2-yl, 3H-,5H-,6H-,7H-isoindazol-2-yl, 4H-,4aH-,5H-isoindazol-2-yl, 1H-,3H-,4aH-,6H-,7H-,7aH-isoindazol-2-yl, 1H-,3H-,5H-,6H-,7H-,7aH-isoindazol-2-yl, 3H-,4H-,4aH-,5H-,6H-,7H-isoindazol-2-yl, 1H-,3H-,4H-,5H-,6H-,7H-isoindazol-2-yl, 4H-,5H-,6H-,7H-isoindazol-2-yl, 1H-,3H-,7H-,7aH-isoindazol-2-yl, 1H-,3H-,4H-,4aH-,5H-,6H-isoindazol-2-yl, 1H-,3H-,4H-, 4aH-,5H-,7H-isoindazol-2-yl, 1H-,7aH-isoindazol-2-yl, 1H-,4H-isoindazol-2-yl, 1H-,4H-, 7H-,7aH-isoindazol-2-yl, 1H-,6H-,7H-,7aH-isoindazol-2-yl, 6H-,7H-isoindazol-2-yl, 1H-, 3H-,6H-,7H-isoindazol-2-yl, 1H-,3H-,4aH-,6H-isoindazol-2-yl, 1H-,4H-,5H-,6H-,7H-,7aH-isoindazol-2-yl,
indoxazin-2-yl, 3H-indoxazin-2-yl, 3H-,4H-,5H-indoxazin-2-yl, 3H-,4H-,7H-indoxazin-2-yl, 3H-,4H-,4aH-,7aH-,7H-indoxazin-2-yl, 3H-,4aH-,7aH-indoxazin-2-yl, 3H-, 5H-,6H-indoxazin-2-yl, 3H-,4aH-,6H-,7H-,7aH-indoxazin-2-yl, 3H-,5H-,6H-,7H-,7aH-indoxazin-2-yl, 3H-,4H-,5H-,6H-,7H-indoxazin-2-yl, 3H-,7H-,7aH-indoxazin-2-yl, 3H-,4H-, 4aH-,5H-,6H-indoxazin-2-yl, 3H-,4H-,4aH-,5H-,7H-indoxazin-2-yl, 7aH-indoxazin-2-yl, 4H-indoxazin-2-yl, 4H-,7H-,7aH-indoxazin-2-yl, 6H-,7H-,7aH-indoxazin-2-yl, 3H-,6H-,7H-indoxazin-2-yl, 3H-,4aH-,6H-indoxazin-2-yl, 4H-,5H-,6H-,7H-,7aH-indoxazin-2-yl,
benzoxazol-3-yl, 4H-,5H-benzoxazol-3-yl, 4H-,7H-benzoxazol-3-yl, 4H-,4aH-,7aH-, 7H-benzoxazol-3-yl, 4aH-,7aH-benzoxazol-3-yl, 5H-,6H-benzoxazol-3-yl, 4aH-,6H-,7H-, 7aH-benzoxazol-3-yl, 5H-,6H-,7H-,7aH-benzoxazol-3-yl, 4H-,5H-,6H-,7H-benzoxazol-3-yl, 7H-,7aH-benzoxazol-3-yl, 4H-,4aH-,5H-,6H-benzoxazol-3-yl, 4H-,4aH-,5H-,7H-benzoxazol-3-yl, 3H-,6H-,7H-benzoxazol-3-yl, 4aH-,6H-benzoxazol-3-yl,
anthran-1-yl, 4aH-,7aH-anthran-1-yl, 3aH-,6H-anthran-1-yl, 3aH-,4H-anthran-1-yl, 6H-,7H-,3aH-,7aH-anthran-1-yl, 4H-,7H-,3aH-,7aH-anthran-1-yl, 4H-,5H-,3aH-,7aH-anthran-1-yl, 4H-,5H-,6H-,3aH-anthran-1-yl, 3H-anthran-1-yl, 3H-,6H-,7H-anthran-1-yl, 3H-,4H-,7H-anthran-1-yl, 3H-,4H-,5H-anthran-1-yl, 3H-,7H-,7aH-anthran-1-yl, 3H-,5H-,7aH-anthran-1-yl, 3H-,5H-,6H-anthran-1-yl, 3H-,4aH-,7aH-anthran-1-yl, 3H-,3aH-,6H-anthran-1-yl, 3H-, 3aH-,4H-anthran-1-yl, 3H-,4H-,5H-,6H-,7H-anthran-1-yl, 3H-,5H-,6H-,7H-,7aH-anthran-1-yl, 3H-,6H-,7H-,3aH-,7aH-anthran-1-yl, 3H-,4H-,7H-,3aH-,7aH-anthran-1-yl, 3H-,4H-,5H-, 3aH-,7aH-anthran-1-yl, 3H-,4H-,5H-,6H-,3 aH-anthran-1-yl,
5H-quinol-1-yl, 2H-quinol-1-yl, 4H-,5H-,6H-,7H-,8H-quinol-1-yl, 2H-,5H-,5aH-,6H-, 7H-,8H-,8aH-quinol-1-yl, 2H-,3H-,5H-,6H-,7H-,8H-,8aH-quinol-1-yl, 4H-,5H-,5Ha-,6H-, 7H-,8H-,8aH-quinol-1-yl, 2H-,3H-,4H-,5H-,6H-,7H-,8H-quinol-1-yl, 8 aH-quinol-1-yl, 7H-, 8H-,8 aH-quinol-1-yl, 5H-,8H-,8 aH-quinol-1-yl, 5H-,6H-,8 aH-quinol-1-yl, 5H-,6H-quinol-1-yl, 5H-,6H-,7H-,8H-,8aH-quinol-1-yl, 2H-,7H-,8H-quinol-1-yl, 2H-,5H-,8H-quinol-1-yl, 2H-, 5H-,6H-quinol-1-yl, 2H-,5H-,6H-,7H-,8H-quinol-1-yl, 4H-,7H-,8H-quinol-1-yl, 4H-,5H-,6H-quinol-1-yl, 4H-quinol-1-yl, 2H-,3H-,7H-,8H-,8 aH-quinol-1-yl, 2H-,3H-,5H-,8H-,8 aH-quinol-1-yl, 2H-,3H-,5H-,6H-,8 aH-quinol-1-yl, 2H-,3H-,5H-,6H-,7H-quinol-1-yl, 2H-,3H-, 5H-quinol-1-yl, 2H-,3H-,7H-quinol-1-yl, 2H-,3H-,8 aH-quinol-1-yl, 2H-,6H-,7H-,8H-,8 aH-quinol-1-yl, 2H-,5H-,5 aH-,8H-,8 aH-quinol-1-yl, 2H-,5H-,5 aH-,6H-quinol-1-yl, 2H-,5H-,5 aH-, 6H-,7H-quinol-1-yl, 2H-,5H-,6H-,7H-,8H-quinol-1-yl, 2H-,7H-,8H-quinol-1-yl, 2H-,5H-,6H-quinol-1-yl, 2H-,5 H-,8 H-quinol-1-yl, 2H-,6H-,7 H-quinol-1-yl, 2H-,5aH-,8 aH-quinol-1-yl, 2H-,6H-,8aH-quinol-1-yl, 2H-,5aH-,7H-quinol-1-yl, 4H-,6H-,7H-,8H-,8aH-quinol-1-yl, 4H-, 5H-,5 aH-,8H-,8 aH-quinol-1-yl, 4H-,5H-,5 aH-,6H-quinol-1-yl, 4H-,5H-,5 aH-,6H-,7H-quinol-1-yl, 4H-,5H-,6H-,7H-,8H-quinol-1-yl, 4H-,7H-,8H-quinol-1-yl, 4H-,5H-,6H-quinol-1-yl, 4H-, 5H-,8H-quinol-1-yl, 4H-,6H-,7H-quinol-1-yl, 4H-,5 aH-,8 aH-quinol-1-yl, 2H-,3H-,4H-,7H-, 8H-quinol-1-yl, 2H-,3H-,4H-,5H-,8H-quinol-1-yl, 2H-,3H-,4H-,5H-,6H-quinol-1-yl, 2H-,3H-, 4H-,6H-,7H-quinol-1-yl, 2H-,3H-,4H-,5aH-,8aH-quinol-1-yl, 2H-,3H-,4H-,5H-,8H-quinol-1-yl, 2H-,3H-,4H-,5aH-,7H-quinol-1-yl, 2H-,3H-,4H-,6H-,8aH-quinol-1-yl, 2H-,3H-,4H-,5H-, 6H-quinol-1-yl, 2H-,3H-,4H-,7H-,8H-quinol-1-yl, 4H-,6H-,8aH-quinol-1-yl, 4H-,5aH-,7H-quinol-1-yl,
1H-isoquinol-2-yl, 1H-,5H-,6H-isoquinol-2-yl, 1H-,5H-,8H-isoquinol-2-yl, 1H-,7H-, 8H-isoquinol-2-yl, 1H-,5H-,6H-,7H-,8H-isoquinol-2-yl,
3H-isoquinol-2-yl, 3H-,5H-,6H-isoquinol-2-yl, 3H-,5H-,8H-isoquinol-2-yl, 3H-,7H-, 8H-isoquinol-2-yl, 3H-,5H-,6H-,7H-,8H-isoquinol-2-yl,
5aH-isoquinol-2-yl, 5H-,5aH-,6H-isoquinol-2-yl, 5H-,5aH-,8H-isoquinol-2-yl, 5aH-, 7H-,8H-isoquinol-2-yl, 6H-,7H-,8H-isoquinol-2-yl, 8H-isoquinol-2-yl, 6H-isoquinol-2-yl, 5H-,5aH-,6H-,7H-,8H-isoquinol-2-yl,
3H-,4H-,5aH-isoquinol-2-yl, 3H-,4H-,6H-isoquinol-2-yl, 3H-,4H-,6H-,7H-,8H-isoquinol-2-yl, 3H-,4H-,5aH-,7H-,8H-isoquinol-2-yl, 3H-,4H-,5H-,5aH-,8H-isoquinol-2-yl, 3H-,4H-,5H-,5aH-,6H-isoquinol-2-yl, 3H-,4H-,8H-isoquinol-2-yl, 3H-,4H-,5H-,5aH-,6H-,7H-, 8H-isoquinol-2-yl,
1H-,3H-,8aH-isoquinol-2-yl, 1H-,3H-,7H-isoquinol-2-yl, 1H-,3H-,5H-isoquinol-2-yl, 1H-,3H-,7H-,8H-,8aH-isoquinol-2-yl, 1H-,3H-,5H-,8H-,8aH-isoquinol-2-yl, 1H-,3H-,5H-,6H-, 8aH-isoquinol-2-yl, 1H-,3H-,5H-,6H-,7H-isoquinol-2-yl, 1H-,3H-,5H-,6H-,7H-,8H-,8aH-isoquinol-2-yl,
1H-,3H-,4H-,7H-,8H-isoquinol-2-yl, 1H-,3H-,4H-,7H-,8H-isoquinol-2-yl, 1H-,3H-, 4H-,5H-,8H-isoquinol-2-yl, 1H-,3H-,4H-,5H-,6H-isoquinol-2-yl, 1H-,3H-,4H-,5H-,6H-,7H-, 8H-isoquinol-2-yl,
1H-,5aH-,8aH-isoquinol-2-yl, 1H-,5aH-,7H-isoquinol-2-yl, 1H-,5aH-,7H-,8H-,8aH-isoquinol-2-yl, 1H-,5H-,5aH-,8H-,8aH-isoquinol-2-yl, 1H-,5H-,5aH-,6H-,8aH-isoquinol-2-yl, 1H-,5H-,5aH-,6H-,7H-isoquinol-2-yl, 1H-,6H-,8aH-isoquinol-2-yl, 1H-,8H-,8aH-isoquinol-2-yl, 1H-,6H-,7H-isoquinol-2-yl, 1H-,5H-,5aH-,6H-,7H-,8H-,8aH-isoquinol-2-yl,
1H-,3H-,4H-,5H-,5aH-,6H-,7H-,8H-,8aH-isoquinol-2-yl, 1H-,3H-,4H-,6H-,7H-,8H-, 8aH-isoquinol-2-yl, 1H-,3H-,4H-,5aH-,7H-,8H-,8aH-isoquinol-2-yl, 1H-,3H-,4H-,5H-,5aH-, 8H-,8aH-isoquinol-2-yl, 1H-,3H-,4H-,5H-,5aH-,6H-,8aH-isoquinol-2-yl, 1H-,3H-,4H-,5H-, 5aH-,6H-,7H-isoquinol-2-yl, 1H-,3H-,4H-,5H-,6H-,7H-,8H-isoquinol-2-yl, 1H-,3H-,4H-,7H-, 8H-isoquinol-2-yl, 1H-,3H-,4H-,5H-,8H-isoquinol-2-yl, 1H-,3H-,4H-,6H-,7H-isoquinol-2-yl, 1H-,3H-,4H-,5H-,6H-isoquinol-2-yl, 1H-,3H-,4H-,5aH-,8aH-isoquinol-2-yl, 1H-,3H-,4H-,8H-, 8aH-isoquinol-2-yl, 1H-,3H-,4H-,6H-,8aH-isoquinol-2-yl, 1H-,3H-,4H-,5aH-,7H-isoquinol-2-yl, 1H-,3H-,4H-,5H-,5aH-isoquinol-2-yl,
5H-cinnol-1-yl, 2H-cinnol-1-yl, 4H-,5H-,6H-,7H-,8H-cinnol-1-yl, 2H-,5H-,5aH-,6H-, 7H-,8H-,8aH-cinnol-1-yl, 2H-,3H-,5H-,6H-,7H-,8H-,8aH-cinnol-1-yl, 4H-,5H-,5Ha-,6H-, 7H-,8H-,8aH-cinnol-1-yl, 2H-,3H-,4H-,5H-,6H-,7H-,8H-cinnol-1-yl, 8aH-cinnol-1-yl, 7H-, 8H-,8aH-cinnol-1-yl, 5H-,8H-,8aH-cinnol-1-yl, 5H-,6H-,8aH-cinnol-1-yl, 5H-,6H-cinnol-1-yl, 5H-,6H-,7H-,8H-,8aH-cinnol-1-yl, 2H-,7H-,8H-cinnol-1-yl, 2H-,5H-,8H-cinnol-1-yl, 2H-, 5H-,6H-cinnol-1-yl, 2H-,5H-,6H-,7H-,8H-cinnol-1-yl, 4H-,7H-,8H-cinnol-1-yl, 4H-,5H-,6H-cinnol-1-yl, 4H-cinnol-1-yl, 2H-,3H-,7H-,8H-,8aH-cinnol-1-yl, 2H-,3H-,5H-,8H-,8aH-cinnol-1-yl, 2H-,3H-,5H-,6H-,8aH-cinnol-1-yl, 2H-,3H-,5H-,6H-,7H-cinnol-1-yl, 2H-,3H-,5H-cinnol-1-yl, 2H-,3H-,7H-cinnol-1-yl, 2H-,3H-,8aH-cinnol-1-yl, 2H-,6H-,7H-,8H-,8aH-cinnol-1-yl, 2H-,5H-,5aH-,8H-,8aH-cinnol-1-yl, 2H-,5H-,5aH-,6H-cinnol-1-yl, 2H-,5H-,5aH-,6H-, 7H-cinnol-1-yl, 2H-,5H-,6H-,7H-,8H-cinnol-1-yl, 2H-,7H-,8H-cinnol-1-yl, 2H-,5H-,6H-cinnol-1-yl, 2H-,5H-,8H-cinnol-1-yl, 2H-,6H-,7H-cinnol-1-yl, 2H-,5 aH-,8 aH-cinnol-1-yl, 2H-, 6H-,8aH-cinnol-1-yl, 2H-,5aH-,7H-cinnol-1-yl, 4H-,6H-,7H-,8H-,8aH-cinnol-1-yl, 4H-,5H-, 5aH-,8H-,8aH-cinnol-1-yl, 4H-,5H-,5aH-,6H-cinnol-1-yl, 4H-,5H-,5aH-,6H-,7H-cinnol-1-yl, 4H-,5H-,6H-,7H-,8H-cinnol-1-yl, 4H-,7H-,8H-cinnol-1-yl, 4H-,5H-,6H-cinnol-1-yl, 4H-,5H-, 8H-cinnol-1-yl, 4H-,6H-,7H-cinnol-1-yl, 4H-,5aH-,8aH-cinnol-1-yl, 2H-,3H-,4H-,7H-,8H-cinnol-1-yl, 2H-,3H-,4H-,5H-,8H-cinnol-1-yl, 2H-,3H-,4H-,5H-,6H-cinnol-1-yl, 2H-,3H-,4H-, 6H-,7H-cinnol-1-yl, 2H-,3H-,4H-,5aH—,8aH-cinnol-1-yl, 2H-,3H-,4H-,5H-,8H-cinnol-1-yl, 2H-,3H-,4H-,5aH-,7H-cinnol-1-yl, 2H-,3H-,4H-,6H-,8aH-cinnol-1-yl, 2H-,3H-,4H-,5H-,6H-cinnol-1-yl, 2H-,3H-,4H-,7H-,8H-cinnol-1-yl, 4H-,6H-,8aH-cinnol-1-yl, 4H-,5aH-,7H-cinnol-1-yl,
1H-cinnol-2-yl, 1H-,5H-,6H-cinnol-2-yl, 1H-,5H-,8H-cinnol-2-yl, 1H-,7H-,8H-cinnol-2-yl, 1H-,5H-,6H-,7H-,8H-cinnol-2-yl,
3H-cinnol-2-yl, 3H-,5H-,6H-cinnol-2-yl, 3H-,5H-,8H-cinnol-2-yl, 3H-,7H-,8H-cinnol-2-yl, 3H-,5H-,6H-,7H-,8H-cinnol-2-yl,
5aH-cinnol-2-yl, 5H-,5aH-,6H-cinnol-2-yl, 5H-,5aH-,8H-cinnol-2-yl, 5aH-,7H-,8H-cinnol-2-yl, 6H-,7H-,8H-cinnol-2-yl, 8H-cinnol-2-yl, 6H-cinnol-2-yl, 5H-,5aH-,6H-,7H-,8H-cinnol-2-yl,
3H-,4H-,5aH-cinnol-2-yl, 3H-,4H-,6H-cinnol-2-yl, 3H-,4H-,6H-,7H-,8H-cinnol-2-yl, 3H-,4H-,5aH-,7H-,8H-cinnol-2-yl, 3H-,4H-,5H-,5aH-,8H-cinnol-2-yl, 3H-,4H-,5H-,5aH-,6H-cinnol-2-yl, 3H-,4H-,8H-cinnol-2-yl, 3H-,4H-,5H-,5aH-,6H-,7H-,8H-cinnol-2-yl,
1H-,3H-,8aH-cinnol-2-yl, 1H-,3H-,7H-cinnol-2-yl, 1H-,3H-,5H-cinnol-2-yl, 1H-,3H-, 7H-,8H-,8aH-cinnol-2-yl, 1H-,3H-,5H-,8H-,8aH-cinnol-2-yl, 1H-,3H-,5H-,6H-,8aH-cinnol-2-yl, 1H-,3H-,5H-,6H-,7H-cinnol-2-yl, 1H-,3H-,5H-,6H-,7H-,8H-,8aH-cinnol-2-yl,
1H-,3H-,4H-,7H-,8H-cinnol-2-yl, 1H-,3H-,4H-,7H-,8H-cinnol-2-yl, 1H-,3H-,4H-,5H-, 8H-cinnol-2-yl, 1H-,3H-,4H-,5H-,6H-cinnol-2-yl, 1H-,3H-,4H-,5H-,6H-,7H-,8H-cinnol-2-yl,
1H-,5aH-,8aH-cinnol-2-yl, 1H-,5aH-,7H-cinnol-2-yl, 1H-,5aH-,7H-,8H-,8aH-cinnol-2-yl, 1H-,5H-,5aH-,8H-,8aH-cinnol-2-yl, 1H-,5H-,5aH-,6H-,8aH-cinnol-2-yl, 1H-,5H-,5aH-, 6H-,7H-cinnol-2-yl, 1H-,6H-,8aH-cinnol-2-yl, 1H-,8H-,8aH-cinnol-2-yl, 1H-,6H-,7H-cinnol-2-yl, 1H-,5H-,5aH-,6H-,7H-,8H-,8aH-cinnol-2-yl,
1H-,3H-,4H-,5H-,5aH-,6H-,7H-,8H-,8aH-cinnol-2-yl, 1H-,3H-,4H-,6H-,7H-,8H-,8aH-cinnol-2-yl, 1H-,3H-,4H-,5aH-,7H-,8H-,8aH-cinnol-2-yl, 1H-,3H-,4H-,5H-,5aH-,8H-,8aH-cinnol-2-yl, 1H-,3H-,4H-,5H-,5aH-,6H-,8aH-cinnol-2-yl, 1H-,3H-,4H-,5H-,5aH-,6H-,7H-cinnol-2-yl, 1H-,3H-,4H-,5H-,6H-,7H-,8H-cinnol-2-yl, 1H-,3H-,4H-,7H-,8H-cinnol-2-yl, 1H-, 3H-,4H-,5H-,8H-cinnol-2-yl, 1H-,3H-,4H-,6H-,7H-cinnol-2-yl, 1H-,3H-,4H-,5H-,6H-cinnol-2-yl, 1H-,3H-,4H-,5aH-,8aH-cinnol-2-yl, 1H-,3H-,4H-,8H-,8aH-cinnol-2-yl, 1H-,3H-,4H-, 6H-,8aH-cinnol-2-yl, 1H-,3H-,4H-,5aH-,7H-cinnol-2-yl, 1H-,3H-,4H-,5H-,5aH-cinnol-2-yl,
5H-quinazol-1-yl, 2H-quinazol-1-yl, 4H-,5H-,6H-,7H-,8H-quinazol-1-yl, 2H-,5H-, 5aH-,6H-,7H-,8H-,8aH-quinazol-1-yl, 2H-,3H-,5H-,6H-,7H-,8H-,8aH-quinazol-1-yl, 4H-, 5H-,5Ha-,6H-,7H-,8H-,8 aH-quinazol-1-yl, 2H-,3H-,4H-,5H-,6H-,7H-,8H-quinazol-1-yl, 8 aH-quinazol-1-yl, 7H-,8H-,8aH-quinazol-1-yl, 5H-,8H-,8 aH-quinazol-1-yl, 5H-,6H-,8aH-quinazol-1-yl, 5H-,6H-quinazol-1-yl, 5H-,6H-,7H-,8H-,8aH-quinazol-1-yl, 2H-,7H-,8H-quinazol-1-yl, 2H-,5H-,8H-quinazol-1-yl, 2H-,5H-,6H-quinazol-1-yl, 2H-,5H-,6H-,7H-,8H-quinazol-1-yl, 4H-,7H-,8H-quinazol-1-yl, 4H-,5H-,6H-quinazol-1-yl, 4H-quinazol-1-yl, 2H-, 3H-,7H-,8H-,8aH-quinazol-1-yl, 2H-,3H-,5H-,8H-,8aH-quinazol-1-yl, 2H-,3H-,5H-,6H-, 8aH-quinazol-1-yl, 2H-,3H-,5H-,6H-,7H-quinazol-1-yl, 2H-,3H-,5H-quinazol-1-yl, 2H-,3H-, 7H-quinazol-1-yl, 2H-,3H-,8 aH-quinazol-1-yl, 2H-,6H-,7H-,8H-,8aH-quinazol-1-yl, 2H-,5H-, 5aH-,8H-,8aH-quinazol-1-yl, 2H-,5H-,5 aH-,6H-quinazol-1-yl, 2H-,5H-,5aH-,6H-,7H-quinazol-1-yl, 2H-,5H-,6H-,7H-,8H-quinazol-1-yl, 2H-,7H-,8H-quinazol-1-yl, 2H-,5H-,6H-quinazol-1-yl, 2H-,5H-,8H-quinazol-1-yl, 2H-,6H-,7H-quinazol-1-yl, 2H-,5 aH-,8 aH-quinazol-1-yl, 2H-,6H-,8 aH-quinazol-1-yl, 2H-,5aH-,7H-quinazol-1-yl, 4H-,6H-,7H-,8H-, 8aH-quinazol-1-yl, 4H-,5H-,5aH-,8H-,8aH-quinazol-1-yl, 4H-,5H-,5aH-,6H-quinazol-1-yl, 4H-,5H-,5aH-,6H-,7H-quinazol-1-yl, 4H-,5H-,6H-,7H-,8H-quinazol-1-yl, 4H-,7H-,8H-quinazol-1-yl, 4H-,5H-,6H-quinazol-1-yl, 4H-,5H-,8H-quinazol-1-yl, 4H-,6H-,7H-quinazol-1-yl, 4H-,5aH-,8aH-quinazol-1-yl, 2H-,3H-,4H-,7H-,8H-quinazol-1-yl, 2H-,3H-,4H-,5H-,8H-quinazol-1-yl, 2H-,3H-,4H-,5H-,6H-quinazol-1-yl, 2H-,3H-,4H-,6H-,7H-quinazol-1-yl, 2H-, 3H-,4H-,5aH-,8 aH-quinazol-1-yl, 2H-,3H-,4H-,5H-,8H-quinazol-1-yl, 2H-,3H-,4H-,5aH-, 7H-quinazol-1-yl, 2H-,3H-,4H-,6H-,8 aH-quinazol-1-yl, 2H-,3H-,4H-,5H-,6H-quinazol-1-yl,
2H-,3H-,4H-,7H-,8H-quinazol-1-yl, 4H-,6H-,8 aH-quinazol-1-yl, 4H-,5aH-,7H-quinazol-1-yl, 2H-,4H-,5H-,5aH-,6H-,7H-,8H-quinazol-3-yl, 2H-,4H-,6H-,7H-,8H-quinazol-3-yl, 2H-,4H-,5aH-,7H-,8H-quinazol-3-yl, 2H-,4H-,5H-,5aH-,8H-quinazol-3-yl, 2H-,4H-,5H-,5aH-, 6H-quinazol-3-yl, 2H-,4H-,8H-quinazol-3-yl, 2H-,4H-,6H-quinazol-3-yl, 2H-,4H-,5aH-quinazol-3-yl, 4H-,5H-,5aH-,6H-,7H-,8H-,8aH-quinazol-3-yl, 4H-,6H-,7H-,8H-,8aH-quinazol-3-yl, 4H-,5aH-,7H-,8H-,8aH-quinazol-3-yl, 4H-,5H-,5aH-,8H-,8aH-quinazol-3-yl, H-,5H-,5aH-,6H-,8aH-quinazol-3-yl, 4H-,5H-,5aH-,6H-,7H-quinazol-3-yl, 4H-,6H-,7H-quinazol-3-yl, 4H-,5aH-,7H-quinazol-3-yl, 4H-,5H-,5aH-quinazol-3-yl, 4H-,6H-,8aH-quinazol-3-yl, 4H-,7H-,8aH-quinazol-3-yl, 4H-,5aH-,8aH-quinazol-3-yl, 2H-,5H-,6H-,7H-, 8H-quinazol-3-yl, 2H-,7H-,8H-quinazol-3-yl, 2H-,5H-,8H-quinazol-3-yl, 2H-,5H-,6H-quinazol-3-yl, 2H-quinazol-3-yl, 5H-,6H-,7H-,8H-,8aH-quinazol-3-yl, 7H-,8H-,8aH-quinazol-3-yl, 5H-,8H-,8aH-quinazol-3-yl, 5H-,6H-,8aH-quinazol-3-yl, 5H-,6H-,7H-quinazol-3-yl, 6H-quinazol-3-yl, 5H-quinazol-3-yl, 8aH-quinazol-3-yl, 4H-,5H-,6H-,7H-,8H-quinazol-3-yl, 4H-,7H-,8H-quinazol-3-yl, 4H-,5H-,8H-quinazol-3-yl, 4H-,5H-,6H-quinazol-3-yl, 4H-quinazol-3-yl, 1H-,2H-,5H-,6H-,7H-,8H-,8aH-quinazol-3-yl, 1H-,2H-,4H-,5H-,6H-, 7H-,8H-quinazol-3-yl, 1H-,2H-,7H-,8H-,8aH-quinazol-3-yl, 1H-,2H-,5H-,8H-,8aH-quinazol-3-yl, 1H-,2H-,5H-,6H-,8aH-quinazol-3-yl, 1H-,2H-,5H-,6H-,7H-quinazol-3-yl, 1H-,2H-,5H-quinazol-3-yl, 1H-,2H-,7H-quinazol-3-yl, 1H-,2H-,8aH-quinazol-3-yl, 1H-,2H-,4H-,7H-,8H-quinazol-3-yl, 1H-,2H-,4H-,5H-,8H-quinazol-3-yl, 1H-,2H-,4H-,5H-,6H-quinazol-3-yl, 1H-, 2H-,4H-,6H-,7H-quinazol-3-yl, 1H-,2H-,4H-,5aH-,8aH-quinazol-3-yl, 1H-,2H-,4H-,5H-,8H-quinazol-3-yl, 1H-,2H-,4H-,5aH-,7H-quinazol-3-yl, 1H-,2H-,4H-,6H-,8aH-quinazol-3-yl, 1H-, 2H-,4H-,5H-,6H-quinazol-3-yl, 1H-,2H-,4H-,7H-,8H-quinazol-3-yl,
5H-naphthyridin-1-yl, 2H-naphthyridin-1-yl, 4H-,5H-,6H-,7H-,8H-naphthyridin-1-yl, 2H-,5H-,5aH-,6H-,7H-,8H-,8aH-naphthyridin-1-yl, 2H-,3H-,5H-,6H-,7H-,8H-,8 aH-naphthyridin-1-yl, 4H-,5H-,5Ha-,6H-,7H-,8H-,8 aH-naphthyridin-1-yl, 2H-,3H-,4H-,5H-,6H-, 7H-,8H-naphthyridin-1-yl, 8 aH-naphthyridin-1-yl, 7H-,8H-,8 aH-naphthyridin-1-yl, 5H-,8H-, 8 aH-naphthyridin-1-yl, 5H-,6H-,8 aH-naphthyridin-1-yl, 5H-,6H-naphthyridin-1-yl, 5H-,6H-, 7H-,8H-,8 aH-naphthyridin-1-yl, 2H-,7H-,8H-naphthyridin-1-yl, 2H-,5H-,8H-naphthyridin-1-yl, 2H-,5H-,6H-naphthyridin-1-yl, 2H-,5H-,6H-,7H-,8H-naphthyridin-1-yl, 4H-,7H-,8H-naphthyridin-1-yl, 4H-,5H-,6H-naphthyridin-1-yl, 4H-naphthyridin-1-yl, 2H-,3H-,7H-,8H-, 8 aH-naphthyridin-1-yl, 2H-,3H-,5H-,8H-,8 aH-naphthyridin-1-yl, 2H-,3H-,5H-,6H-,8 aH-naphthyridin-1-yl, 2H-,3H-,5H-,6H-,7H-naphthyridin-1-yl, 2H-,3H-,5H-naphthyridin-1-yl, 2H-,3H-,7H-naphthyridin-1-yl, 2H-,3H-,8 aH-naphthyridin-1-yl, 2H-,6H-,7H-,8H-,8 aH-naphthyridin-1-yl, 2H-,5H-,5aH-,8H-,8 aH-naphthyridin-1-yl, 2H-,5H-,5aH-,6H-naphthyridin-1-yl, 2H-,5H-,5aH-,6H-,7H-naphthyridin-1-yl, 2H-,5H-,6H-,7H-,8H-naphthyridin-1-yl, 2H-, 7H-,8H-naphthyridin-1-yl, 2H-,5H-,6H-naphthyridin-1-yl, 2H-,5H-,8H-naphthyridin-1-yl, 2H-,6H-,7H-naphthyridin-1-yl, 2H-,5 aH-,8 aH-naphthyridin-1-yl, 2H-,6H-,8 aH-naphthyridin-1-yl, 2H-,5aH-,7H-naphthyridin-1-yl, 4H-,6H-,7H-,8H-,8 aH-naphthyridin-1-yl, 4H-,5H-,5aH-, 8H-,8aH-naphthyridin-1-yl, 4H-,5H-,5aH-,6H-naphthyridin-1-yl, 4H-,5H-,5aH-,6H-,7H-naphthyridin-1-yl, 4H-,5H-,6H-,7H-,8H-naphthyridin-1-yl, 4H-,7H-,8H-naphthyridin-1-yl, 4H-,5H-,6H-naphthyridin-1-yl, 4H-,5H-,8H-naphthyridin-1-yl, 4H-,6H-,7H-naphthyridin-1-yl, 4H-,5aH-,8 aH-naphthyridin-1-yl, 2H-,3H-,4H-,7H-,8H-naphthyridin-1-yl, 2H-,3H-,4H-, 5H-,8H-naphthyridin-1-yl, 2H-,3H-,4H-,5H-,6H-naphthyridin-1-yl, 2H-,3H-,4H-,6H-,7H-naphthyridin-1-yl, 2H-,3H-,4H-,5aH-,8 aH-naphthyridin-1-yl, 2H-,3H-,4H-,5H-,8H-naphthyridin-1-yl, 2H-,3H-,4H-,5aH-,7H-naphthyridin-1-yl, 2H-,3H-,4H-,6H-,8 aH-naphthyridin-1-yl, 2H-,3 H-,4H-,5 H-,6H-naphthyridin-1-yl, 2H-,3 H-,4H-,7 H-,8 H-naphthyridin-1-yl, 4H-,6H-,8 aH-naphthyridin-1-yl, 4H-,5aH-,7 H-naphthyridin-1-yl,
5H-pyrido[3,4-b]pyridin-1-yl, 2H-pyrido[3,4-b]pyridin-1-yl, 4H-,5H-,6H-,7H-,8H-pyrido[3,4-b]pyridin-1-yl, 2H-,5H-,5aH-,6H-,7H-,8H-,8 aH-pyrido[3,4-b]pyridin-1-yl, 2H-, 3H-,5H-,6H-,7H-,8H-,8aH-pyrido[3,4-b]pyridin-1-yl, 4H-,5H-,5Ha-,6H-,7H-,8H-,8aH-pyrido[3,4-b]pyridin-1-yl, 2H-,3H-,4H-,5H-,6H-,7H-,8H-pyrido[3,4-b]pyridin-1-yl, 8 aH-pyrido[3,4-b]pyridin-1-yl, 7H-,8H-,8 aH-pyrido[3,4-b]pyridin-1-yl, 5H-,8H-,8 aH-pyrido[3,4-b]pyridin-1-yl, 5 H-,6H-,8 aH-pyrido[3,4-b]pyridin-1-yl, 5 H-,6H-pyrido[3,4-b]pyridin-1-yl, 5H-,6H-,7H-,8H-,8aH-pyrido[3,4-b]pyridin-1-yl, 2H-,7H-,8H-pyrido[3,4-b]pyridin-1-yl, 2H-, 5H-,8H-pyrido[3,4-b]pyridin-1-yl, 2H-,5H-,6H-pyrido[3,4-b]pyridin-1-yl, 2H-,5H-,6H-,7H-, 8H-pyrido[3,4-b]pyridin-1-yl, 4H-,7H-,8H-pyrido[3,4-b]pyridin-1-yl, 4H-,5H-,6H-pyrido[3,4-b]pyridin-1-yl, 4H-pyrido[3,4-b]pyridin-1-yl, 2H-,3H-,7H-,8H-,8 aH-pyrido[3,4-b]pyridin-1-yl, 2H-,3H-,5H-,8H-,8 aH-pyrido[3,4-b]pyridin-1-yl, 2H-,3H-,5H-,6H-,8 aH-pyrido[3,4-b]pyridin-1-yl, 2H-,3H-,5H-,6H-,7H-pyrido[3,4-b]pyridin-1-yl, 2H-,3H-,5H-pyrido[3,4-b]pyridin-1-yl, 2H-,3H-,7H-pyrido[3,4-b]pyridin-1-yl, 2H-,3H-,8 aH-pyrido[3,4-b]pyridin-1-yl, 2H-,6H-,7H-,8H-,8 aH-pyrido[3,4-b]pyridin-1-yl, 2H-,5H-,5aH-,8H-,8 aH-pyrido[3,4-b]pyridin-1-yl, 2H-,5H-,5aH-,6H-pyrido[3,4-b]pyridin-1-yl, 2H-,5H-,5aH-,6H-, 7H-pyrido[3,4-b]pyridin-1-yl, 2H-,5H-,6H-,7H-,8H-pyrido[3,4-b]pyridin-1-yl, 2H-,7H-,8H-pyrido[3,4-b]pyridin-1-yl, 2H-,5H-,6H-pyrido[3,4-b]pyridin-1-yl, 2H-,5H-,8H-pyrido[3,4-b]pyridin-1-yl, 2H-,6H-,7H-pyrido[3,4-b]pyridin-1-yl, 2H-,5aH-,8 aH-pyrido[3,4-b]pyridin-1-yl, 2H-,6H-,8aH-pyrido[3,4-b]pyridin-1-yl, 2H-,5aH-,7H-pyrido[3,4-b]pyridin-1-yl, 4H-,6H-, 7H-,8H-,8aH-pyrido[3,4-b]pyridin-1-yl, 4H-,5 H-,5 aH-,8 H-,8 aH-pyrido[3,4-b]pyridin-1-yl, 4H-,5H-,5 aH-,6H-pyrido[3,4-b]pyridin-1-yl, 4H-,5H-,5 aH-,6H-,7 H-pyrido[3,4-b]pyridin-1-yl, 4H-,5H-,6H-,7H-,8H-pyrido[3,4-b]pyridin-1-yl, 4H-,7H-,8H-pyrido[3,4-b]pyridin-1-yl, 4H-, 5H-,6H-pyrido[3,4-b]pyridin-1-yl, 4H-,5H-,8H-pyrido[3,4-b]pyridin-1-yl, 4H-,6H-,7H-pyrido[3,4-b]pyridin-1-yl, 4H-,5aH-,8 aH-pyrido[3,4-b]pyridin-1-yl, 2H-,3H-,4H-,7H-,8H-pyrido[3,4-b]pyridin-1-yl, 2H-,3H-,4H-,5H-,8H-pyrido[3,4-b]pyridin-1-yl, 2H-,3H-,4H-,5H-, 6H-pyrido[3,4-b]pyridin-1-yl, 2H-,3H-,4H-,6H-,7H-pyrido[3,4-b]pyridin-1-yl, 2H-,3H-,4H-, 5aH-,8 aH-pyrido[3,4-b]pyridin-1-yl, 2H-,3H-,4H-,5H-,8H-pyrido[3,4-b]pyridin-1-yl, 2H-,3H-,4H-,5aH-,7H-pyrido[3,4-b]pyridin-1-yl, 2H-,3H-,4H-,6H-,8 aH-pyrido[3,4-b]pyridin-1-yl, 2H-,3H-,4H-,5H-,6H-pyrido[3,4-b]pyridin-1-yl, 2H-,3H-,4H-,7H-,8H-pyrido[3,4-b]pyridin-1-yl, 4H-,6H-,8 aH-pyrido[3,4-b]pyridin-1-yl, 4H-,5aH-,7H-pyrido[3,4-b]pyridin-1-yl,
2H-,3H-,4H-,5H-,5aH-,6H-,8H-pyrido[3,4-b]pyridin-7-yl, 2H-,3H-,4H-,5H-,6H-,8H-pyrido[3,4-b]pyridin-7-yl, 2H-,3H-,4H-,5aH-,8H-pyrido[3,4-b]pyridin-7-yl, 2H-,3H-,4H-,5H-, 5aH-,6H-pyrido[3,4-b]pyridin-7-yl, 3H-,4H-,5H-,5aH-,6H-,8H-,8aH-pyrido[3,4-b]pyridin-7-yl, 3H-,4H-,6H-,8H-,8aH-pyrido[3,4-b]pyridin-7-yl, 3H-,4H-,5aH-,8H-,8aH-pyrido[3,4-b]pyridin-7-yl, 3H-,4H-,5H-,5aH-,6H-,8aH-pyrido[3,4-b]pyridin-7-yl, 3H-,4H-,5H-,5aH-,6H-pyrido[3,4-b]pyridin-7-yl, 3H-,4H-,6H-pyrido[3,4-b]pyridin-7-yl, 3H-,4H-,5aH-pyrido[3,4-b]pyridin-7-yl, 4H-,5H-,5aH-,6H-,8H-pyrido[3,4-b]pyridin-7-yl, 4H-,6H-,8H-pyrido[3,4-b]pyridin-7-yl, 4H-,8H-pyrido[3,4-b]pyridin-7-yl, 3H-,4H-,5H-,6H-,8H-pyrido[3,4-b]pyridin-7-yl, 3H-,4H-,8H-pyrido[3,4-b]pyridin-7-yl, 2H-,5H-,5aH-,6H-,8H-pyrido[3,4-b]pyridin-7-yl, 2H-,6H-,8H-pyrido[3,4-b]pyridin-7-yl, 2H-,8H-pyrido[3,4-b]pyridin-7-yl, 3H-,5H-,6H-,8H-, 8aH-pyrido[3,4-b]pyridin-7-yl, 3H-,8H-,8aH-pyrido[3,4-b]pyridin-7-yl, 3H-pyrido[3,4-b]pyridin-7-yl, 5H-,6H-,8H-pyrido[3,4-b]pyridin-7-yl, 8H-pyrido[3,4-b]pyridin-7-yl, 1H-,4H-, 5H-,6H-,8H-pyrido[3,4-b]pyridin-7-yl, 1H-,2H-,5H-,5aH-,6H-,8H-,8aH-pyrido[3,4-b]pyridin-7-yl, 1H-,2H-,3H-,5H-,6H-,8H-,8aH-pyrido[3,4-b]pyridin-7-yl, 1H-,4H-,5H-,5Ha-, 6H-,8H-,8aH-pyrido[3,4-b]pyridin-7-yl, 1H-,2H-,3H-,4H-,5H-,6H-,8H-pyrido[3,4-b]pyridin-7-yl, 1H-,8H-,8aH-pyrido[3,4-b]pyridin-7-yl, 1H-,5H-,6H-,8H-,8aH-pyrido[3,4-b]pyridin-7-yl, 1H-,2H-,8H-pyrido[3,4-b]pyridin-7-yl, 1H-,2H-,5H-,6H-,8H-pyrido[3,4-b]pyridin-7-yl, 1H-,4H-,8H-pyrido[3,4-b]pyridin-7-yl, 1H-,2H-,3H-,8H-,8aH-pyrido[3,4-b]pyridin-7-yl, 1H-, 2H-,3H-,5H-,8H-,8aH-pyrido[3,4-b]pyridin-7-yl, 1H-,2H-,3H-,5H-,6H-pyrido[3,4-b]pyridin-7-yl, 1H-,2H-,3H-pyrido[3,4-b]pyridin-7-yl, 1H-,2H-,6H-,8H-,8aH-pyrido[3,4-b]pyridin-7-yl, 1H-,2H-,5H-,5aH-,6H-pyrido[3,4-b]pyridin-7-yl, 1H-,2H-,5H-,6H-,8H-pyrido[3,4-b]pyridin-7-yl, 1H-,2H-,8H-pyrido[3,4-b]pyridin-7-yl, 1H-,2H-,6H-pyrido[3,4-b]pyridin-7-yl, 1H-,2H-, 5aH-pyrido[3,4-b]pyridin-7-yl, 1H-,4H-,6H-,8H-,8aH-pyrido[3,4-b]pyridin-7-yl, 1H-,4H-, 5H-,5aH-,6H-pyrido[3,4-b]pyridin-7-yl, 1H-,4H-,5H-,6H-,8H-pyrido[3,4-b]pyridin-7-yl, 1H-, 4H-,8H-pyrido[3,4-b]pyridin-7-yl, 1H-,4H-,6H-pyrido[3,4-b]pyridin-7-yl, 1H-,2H-,3H-,4H-, 8H-pyrido[3,4-b]pyridin-7-yl, 1H-,2H-,3H-,4H-,6H-pyrido[3,4-b]pyridin-7-yl, 1H-,2H-,3H-, 4H-,5aH-,8aH-pyrido[3,4-b]pyridin-7-yl, 1H-,2H-,3H-,4H-,5aH-pyrido[3,4-b]pyridin-7-yl, 1H-,2H-,3H-,4H-,8H-pyrido[3,4-b]pyridin-7-yl, 1H-,4H-,5aH-pyrido[3,4-b]pyridin-7-yl,
5H-pyrido[3,2-b]pyridin-1-yl, 2H-pyrido[3,2-b]pyridin-1-yl, 4H-,5H-,6H-,7H-,8H-pyrido[3,2-b]pyridin-1-yl, 2H-,5H-,5aH-,6H-,7H-,8H-,8aH-pyrido[3,2-b]pyridin-1-yl, 2H-, 3H-,5H-,6H-,7H-,8H-,8aH-pyrido[3,2-b]pyridin-1-yl, 4H-,5H-,5Ha-,6H-,7H-,8H-,8aH-pyrido[3,2-b]pyridin-1-yl, 2H-,3H-,4H-,5H-,6H-,7H-,8H-pyrido[3,2-b]pyridin-1-yl, 8aH-pyrido[3,2-b]pyridin-1-yl, 7H-,8H-,8aH-pyrido[3,2-b]pyridin-1-yl, 5H-,8H-,8aH-pyrido[3,2-b]pyridin-1-yl, 5H-,6H-,8 aH-pyrido[3,2-b]pyridin-1-yl, 5H-,6H-pyrido[3,2-b]pyridin-1-yl, 5H-,6H-,7H-,8H-,8aH-pyrido[3,2-b]pyridin-1-yl, 2H-,7H-,8H-pyrido[3,2-b]pyridin-1-yl, 2H-, 5H-,8H-pyrido[3,2-b]pyridin-1-yl, 2H-,5H-,6H-pyrido[3,2-b]pyridin-1-yl, 2H-,5H-,6H-,7H-, 8H-pyrido[3,2-b]pyridin-1-yl, 4H-,7H-,8H-pyrido[3,2-b]pyridin-1-yl, 4H-,5H-,6H-pyrido[3,2-b]pyridin-1-yl, 4H-pyrido[3,2-b]pyridin-1-yl, 2H-,3H-,7H-,8H-,8 aH-pyrido[3,2-b]pyridin-1-yl, 2H-,3H-,5H-,8H-,8 aH-pyrido[3,2-b]pyridin-1-yl, 2H-,3H-,5H-,6H-,8 aH-pyrido[3,2-b]pyridin-1-yl, 2H-,3H-,5H-,6H-,7H-pyrido[3,2-b]pyridin-1-yl, 2H-,3H-,5H-pyrido[3,2-b]pyridin-1-yl, 2H-,3H-,7H-pyrido[3,2-b]pyridin-1-yl, 2H-,3H-,8 aH-pyrido[3,2-b]pyridin-1-yl, 2H-,6H-,7H-,8H-,8 aH-pyrido[3,2-b]pyridin-1-yl, 2H-,5H-,5aH-,8H-,8 aH-pyrido[3,2-b]pyridin-1-yl, 2H-,5H-,5aH-,6H-pyrido[3,2-b]pyridin-1-yl, 2H-,5H-,5aH-,6H-, 7H-pyrido[3,2-b]pyridin-1-yl, 2H-,5H-,6H-,7H-,8H-pyrido[3,2-b]pyridin-1-yl, 2H-,7H-,8H-pyrido[3,2-b]pyridin-1-yl, 2H-,5H-,6H-pyrido[3,2-b]pyridin-1-yl, 2H-,5H-,8H-pyrido[3,2-b]pyridin-1-yl, 2H-,6H-,7H-pyrido[3,2-b]pyridin-1-yl, 2H-,5aH-,8 aH-pyrido[3,2-b]pyridin-1-yl, 2H-,6H-,8aH-pyrido[3,2-b]pyridin-1-yl, 2H-,5aH-,7H-pyrido[3,2-b]pyridin-1-yl, 4H-,6H-, 7H-,8H-,8aH-pyrido[3,2-b]pyridin-1-yl, 4H-,5 H-,5aH-,8 H-,8 aH-pyrido[3,2-b]pyridin-1-yl, 4H-,5H-,5aH-,6H-pyrido[3,2-b]pyridin-1-yl, 4H-,5H-,5aH-,6H-,7H-pyrido[3,2-b]pyridin-1-yl, 4H-,5H-,6H-,7H-,8H-pyrido[3,2-b]pyridin-1-yl, 4H-,7H-,8H-pyrido[3,2-b]pyridin-1-yl, 4H-, 5H-,6H-pyrido[3,2-b]pyridin-1-yl, 4H-,5H-,8H-pyrido[3,2-b]pyridin-1-yl, 4H-,6H-,7H-pyrido[3,2-b]pyridin-1-yl, 4H-,5aH-,8 aH-pyrido[3,2-b]pyridin-1-yl, 2H-,3H-,4H-,7H-,8H-pyrido[3,2-b]pyridin-1-yl, 2H-,3H-,4H-,5H-,8H-pyrido[3,2-b]pyridin-1-yl, 2H-,3H-,4H-,5H-, 6H-pyrido[3,2-b]pyridin-1-yl, 2H-,3H-,4H-,6H-,7H-pyrido[3,2-b]pyridin-1-yl, 2H-,3H-,4H-, 5aH-,8 aH-pyrido[3,2-b]pyridin-1-yl, 2H-,3H-,4H-,5H-,8H-pyrido[3,2-b]pyridin-1-yl, 2H-, 3H-,4H-,5aH-,7H-pyrido[3,2-b]pyridin-1-yl, 2H-,3H-,4H-,6H-,8 aH-pyrido[3,2-b]pyridin-1-yl, 2H-,3H-,4H-,5H-,6H-pyrido[3,2-b]pyridin-1-yl, 2H-,3H-,4H-,7H-,8H-pyrido[3,2-b]pyridin-1-yl, 4H-,6H-,8 aH-pyrido[3,2-b]pyridin-1-yl, 4H-,5aH-,7H-pyrido[3,2-b]pyridin-1-yl,
5H-pyrido[4,3-b]pyridin-1-yl, 2H-pyrido[4,3-b]pyridin-1-yl, 4H-,5H-,6H-,7H-,8H-pyrido[4,3-b]pyridin-1-yl, 2H-,5H-,5aH-,6H-,7H-,8H-,8 aH-pyrido[4,3-b]pyridin-1-yl, 2H-, 3H-,5H-,6H-,7H-,8H-,8aH-pyrido[4,3-b]pyridin-1-yl, 4H-,5H-,5Ha-,6H-,7H-,8H-,8aH-pyrido[4,3-b]pyridin-1-yl, 2H-,3H-,4H-,5H-,6H-,7H-,8H-pyrido[4,3-b]pyridin-1-yl, 8 aH-pyrido[4,3-b]pyridin-1-yl, 7H-,8H-,8 aH-pyrido[4,3-b]pyridin-1-yl, 5H-,8H-,8 aH-pyrido[4,3-b]pyridin-1-yl, 5 H-,6H-,8 aH-pyrido[4,3-b]pyridin-1-yl, 5 H-,6H-pyrido[4,3-b]pyridin-1-yl, 5H-,6H-,7H-,8H-,8aH-pyrido[4,3-b]pyridin-1-yl, 2H-,7H-,8H-pyrido[4,3-b]pyridin-1-yl, 2H-, 5H-,8H-pyrido[4,3-b]pyridin-1-yl, 2H-,5H-,6H-pyrido[4,3-b]pyridin-1-yl, 2H-,5H-,6H-,7H-, 8H-pyrido[4,3-b]pyridin-1-yl, 4H-,7H-,8H-pyrido[4,3-b]pyridin-1-yl, 4H-,5H-,6H-pyrido[4,3-b]pyridin-1-yl, 4H-pyrido[4,3-b]pyridin-1-yl, 2H-,3H-,7H-,8H-,8 aH-pyrido[4,3-b]pyridin-1-yl, 2H-,3H-,5H-,8H-,8 aH-pyrido[4,3-b]pyridin-1-yl, 2H-,3H-,5H-,6H-,8 aH-pyrido[4,3-b]pyridin-1-yl, 2H-,3H-,5H-,6H-,7H-pyrido[4,3-b]pyridin-1-yl, 2H-,3H-,5H-pyrido[4,3-b]pyridin-1-yl, 2H-,3H-,7H-pyrido[4,3-b]pyridin-1-yl, 2H-,3H-,8 aH-pyrido[4,3-b]pyridin-1-yl, 2H-,6H-,7H-,8H-,8 aH-pyrido[4,3-b]pyridin-1-yl, 2H-,5H-,5aH-,8H-,8 aH-pyrido[4,3-b]pyridin-1-yl, 2H-,5H-,5aH-,6H-pyrido[4,3-b]pyridin-1-yl, 2H-,5H-,5aH-,6H-, 7H-pyrido[4,3-b]pyridin-1-yl, 2H-,5H-,6H-,7H-,8H-pyrido[4,3-b]pyridin-1-yl, 2H-,7H-,8H-pyrido[4,3-b]pyridin-1-yl, 2H-,5H-,6H-pyrido[4,3-b]pyridin-1-yl, 2H-,5H-,8H-pyrido[4,3-b]pyridin-1-yl, 2H-,6H-,7H-pyrido[4,3-b]pyridin-1-yl, 2H-,5aH-,8 aH-pyrido[4,3-b]pyridin-1-yl, 2H-,6H-,8aH-pyrido[4,3-b]pyridin-1-yl, 2H-,5aH-,7H-pyrido[4,3-b]pyridin-1-yl, 4H-,6H-, 7H-,8H-,8aH-pyrido[4,3-b]pyridin-1-yl, 4H-,5 H-,5aH-,8 H-,8 aH-pyrido[4,3-b]pyridin-1-yl, 4H-,5H-,5 aH-,6H-pyrido[4,3-b]pyridin-1-yl, 4H-,5H-,5 aH-,6H-,7 H-pyrido[4,3-b]pyridin-1-yl, 4H-,5H-,6H-,7H-,8H-pyrido[4,3-b]pyridin-1-yl, 4H-,7H-,8H-pyrido[4,3-b]pyridin-1-yl, 4H-, 5H-,6H-pyrido[4,3-b]pyridin-1-yl, 4H-,5H-,8H-pyrido[4,3-b]pyridin-1-yl, 4H-,6H-,7H-pyrido[4,3-b]pyridin-1-yl, 4H-,5aH-,8 aH-pyrido[4,3-b]pyridin-1-yl, 2H-,3H-,4H-,7H-,8H-pyrido[4,3-b]pyridin-1-yl, 2H-,3H-,4H-,5H-,8H-pyrido[4,3-b]pyridin-1-yl, 2H-,3H-,4H-,5H-, 6H-pyrido[4,3-b]pyridin-1-yl, 2H-,3H-,4H-,6H-,7H-pyrido[4,3-b]pyridin-1-yl, 2H-,3H-,4H-, 5aH-,8 aH-pyrido[4,3-b]pyridin-1-yl, 2H-,3H-,4H-,5H-,8H-pyrido[4,3-b]pyridin-1-yl, 2H-, 3H-,4H-,5aH-,7H-pyrido[4,3-b]pyridin-1-yl, 2H-,3H-,4H-,6H-,8 aH-pyrido[4,3-b]pyridin-1-yl, 2H-,3H-,4H-,5H-,6H-pyrido[4,3-b]pyridin-1-yl, 2H-,3H-,4H-,7H-,8H-pyrido[4,3-b]pyridin-1-yl, 4H-,6H-,8 aH-pyrido[4,3-b]pyridin-1-yl, 4H-,5aH-,7H-pyrido[4,3-b]pyridin-1-yl,
2H-,3H-,4H-,5H-,5aH-,7H-,8H-pyrido[4,3-b]pyridin-6-yl, 2H-,3H-,4H-,7H-,8H-pyrido[4,3-b]pyridin-6-yl, 2H-,3H-,4H-,5H-,5aH-,7H-pyrido[4,3-b]pyridin-6-yl, 2H-,3H-,4H-pyrido[4,3-b]pyridin-6-yl, 3H-,4H-,5H-,5aH-,7H-,8H-,8aH-pyrido[4,3-b]pyridin-6-yl, 3H-, 4H-,7H-,8H-,8aH-pyrido[4,3-b]pyridin-6-yl, 3H-,4H-,5H-,5aH-,7H-,8aH-pyrido[4,3-b]pyridin-6-yl, 3H-,4H-,5H-,5aH-,7H-pyrido[4,3-b]pyridin-6-yl, 3H-,4H-,7H-pyrido[4,3-b]pyridin-6-yl, 3H-,4H-,8aH-pyrido[4,3-b]pyridin-6-yl, 4H-,5H-,5aH-,7H-,8H-pyrido[4,3-b]pyridin-6-yl, 4H-,7H-,8H-pyrido[4,3-b]pyridin-6-yl, 4H-,5H-pyrido[4,3-b]pyridin-6-yl, 4H-pyrido[4,3-b]pyridin-6-yl, 3H-,4H-,5H-,7H-,8H-pyrido[4,3-b]pyridin-6-yl, 3H-,4H-,5H-pyrido[4,3-b]pyridin-6-yl, 2H-,5H-,5aH-,7H-,8H-pyrido[4,3-b]pyridin-6-yl, 2H-,7H-,8H-pyrido[4,3-b]pyridin-6-yl, 2H-,5H-pyrido[4,3-b]pyridin-6-yl, 2H-pyrido[4,3-b]pyridin-6-yl, 3H-,5H-,7H-,8H-,8aH-pyrido[4,3-b]pyridin-6-yl, 3H-,5H-,8aH-pyrido[4,3-b]pyridin-6-yl, 3H-, 5H-pyrido[4,3-b]pyridin-6-yl, 5H-,7H-,8H-pyrido[4,3-b]pyridin-6-yl, 5H-pyrido[4,3-b]pyridin-6-yl, 1H-,4H-,5H-,7H-,8H-pyrido[4,3-b]pyridin-6-yl, 1H-,2H-,3H-,5H-,7H-,8H-, 8aH-pyrido[4,3-b]pyridin-6-yl, 1H-,4H-,5H-,5Ha-,7H-,8H-,8aH-pyrido[4,3-b]pyridin-6-yl, 1H-,2H-,3H-,4H-,5H-,7H-,8H-pyrido[4,3-b]pyridin-6-yl, 1H-,5H-,8aH-pyrido[4,3-b]pyridin-6-yl, 1H-,5H-pyrido[4,3-b]pyridin-6-yl, 1H-,5H-,7H-,8H-,8aH-pyrido[4,3-b]pyridin-6-yl, 1H-, 2H-,5H-pyrido[4,3-b]pyridin-6-yl, 1H-,2H-,5H-,7H-,8H-pyrido[4,3-b]pyridin-6-yl, 1H-,4H-, 5H-pyrido[4,3-b]pyridin-6-yl, 1H-,2H-,3H-,5H-,8aH-pyrido[4,3-b]pyridin-6-yl, 1H-,2H-,3H-, 5H-,7H-pyrido[4,3-b]pyridin-6-yl, 1H-,2H-,7H-,8H-,8aH-pyrido[4,3-b]pyridin-6-yl, 1H-,2H-, 5H-,5aH-pyrido[4,3-b]pyridin-6-yl, 1H-,2H-,5H-,5aH-,7H-pyrido[4,3-b]pyridin-6-yl, 1H-, 2H-,5H-,7H-,8H-pyrido[4,3-b]pyridin-6-yl, 1H-,2H-,5H-pyrido[4,3-b]pyridin-6-yl, 1H-,2H-, 5H-,8H-pyrido[4,3-b]pyridin-6-yl, 1H-,2H-,7H-pyrido[4,3-b]pyridin-6-yl, 1H-,2H-,8aH-pyrido[4,3-b]pyridin-6-yl, 1H-,4H-,7H-,8H-,8aH-pyrido[4,3-b]pyridin-6-yl, 1H-,4H-,5H-, 5aH-pyrido[4,3-b]pyridin-6-yl, 1H-,4H-,5H-,5aH-,7H-pyrido[4,3-b]pyridin-6-yl, 1H-,4H-, 5H-,7H-,8H-pyrido[4,3-b]pyridin-6-yl, 1H-,4H-,5H-pyrido[4,3-b]pyridin-6-yl, 1H-,4H-,7H-pyrido[4,3-b]pyridin-6-yl, 1H-,2H-,3H-,4H-,5H-pyrido[4,3-b]pyridin-6-yl, 1H-,2H-,3H-,4H-, 7H-pyrido[4,3-b]pyridin-6-yl, 1H-,2H-,3H-,4H-,8aH-pyrido[4,3-b]pyridin-6-yl, 1H-,2H-,3H-, 4H-,5H-pyrido[4,3-b]pyridin-6-yl, 1H-,4H-,8aH-pyrido[4,3-b]pyridin-6-yl,
2H-,4H-(1,3-benzoxazin-3-yl), 2H-,4H-,5H-,8H-(1,3-benzoxazin-3-yl), 2H-,4H-,7H-, 8H-(1,3-benzoxazin-3-yl), 2H-,4H-,5H-,6H-(1,3-benzoxazin-3-yl), 2H-,4H-,5H-,6H-,7H-, 8H-(1,3-benzoxazin-3-yl), 2H-,8aH-(1,3-benzoxazin-3-yl), 2H-,5H-(1,3-benzoxazin-3-yl), 2H-,5H-,6H-,7H-(1,3-benzoxazin-3-yl), 2H-,5H-,8H-,8aH-(1,3-benzoxazin-3-yl), 2H-,7H-, 8H-,8aH-(1,3-benzoxazin-3-yl), 2H-,7H-,8H-,8aH-(1,3-benzoxazin-3-yl), 2H-,7H-(1,3-benzoxazin-3-yl), 2H-,5H-,6H-,7H-,8H-,8aH-(1,3-benzoxazin-3-yl), 2H-,4H-,5aH-,8aH-(1,3-benzoxazin-3-yl), 2H-,4H-,5aH-,7H-(1,3-benzoxazin-3-yl), 2H-,4H-,6H-(1,3-benzoxazin-3-yl), 2H-,4H-,6H-,7H-,8H-,8aH-(1,3-benzoxazin-3-yl), 2H-,4H-,5aH-,7H-,8H-,8aH-(1,3-benzoxazin-3-yl), 2H-,4H-,5H-,5aH-,8H-,8aH-1,3-benzoxazin-3-yl), 2H-,4H-,5H-,5aH-,6H-, 8aH-(1,3-benzoxazin-3-yl), 2H-,4H-,5H-,5aH-,6H-,7H-(1,3-benzoxazin-3-yl), 2H-,4H-,5H-, 5aH-,6H-,7H-,8H-,8aH-(1,3-benzoxazin-3-yl),
5H-,5aH-,6H-,7H-,8H-,8aH-(1,4-benzoxazin-4-yl), 6H-,7H-,8H-,8aH-(1,4-benzoxazin-4-yl), 5aH-,7H-,8H-,8aH-(1,4-benzoxazin-4-yl), 5H-,5aH-,8H-,8aH-(1,4-benzoxazin-4-yl), 5H-,5aH-,6H-,8aH-(1,4-benzoxazin-4-yl), 5H-,5aH-,6H-,7H-(1,4-benzoxazin-4-yl), 5aH-,7H-(1,4-benzoxazin-4-yl), 6H-,8aH-(1,4-benzoxazin-4-yl), 8H-,8aH-(1,4-benzoxazin-4-yl), 5H-,5aH-(1,4-benzoxazin-4-yl), 2H-,3H-,5H-,6H-,7H-,8H-(1,4-benzoxazin-4-yl), 2H-,3H-,7H-,8H-(1,4-benzoxazin-4-yl), 2H-,3H-,5H-,8H-(1,4-benzoxazin-4-yl), 2H-,3H-,5H-,6H-(1,4-benzoxazin-4-yl), 5H-,6H-,7H-,8H-(1,4-benzoxazin-4-yl), 7H-, 8H-(1,4-benzoxazin-4-yl), 5H-,8H-(1,4-benzoxazin-4-yl), 5H-,6H-(1,4-benzoxazin-4-yl), 1,4-benzoxazin-4-yl, 2H-,3H-,5H-,5aH-,6H-,7H-,8H-,8aH-(1,4-benzoxazin-4-yl), 2H-,3H-, 6H-,7H-,8H-,8aH-(1,4-benzoxazin-4-yl), 2H-,3H-,5aH-,7H-,8H-,8aH-(1,4-benzoxazin-4-yl), 2H-,3H-,5H-,5aH-,8H-,8aH-(1,4-benzoxazin-4-yl), 2H-,3H-,5H-,5aH-,6H-,8H-(1,4-benzoxazin-4-yl), 2H-,3H-,5H-,5aH-,6H-,7H-(1,4-benzoxazin-4-yl), 2H-,3H-,6H-,7H-(1,4-benzoxazin-4-yl), 2H-,3H-,5aH-,7H-(1,4-benzoxazin-4-yl), 2H-,3H-,5H-,5aH-(1,4-benzoxazin-4-yl), 2H-,3H-,6H-,8aH-(1,4-benzoxazin-4-yl), 2H-,3H-,8H-,8aH-(1,4-benzoxazin-4-yl),
5H-,5aH-,6H-,7H-,8H-,8aH-(2,3-benzoxazin-3-yl), 5aH-,7H-,8H-(2,3-benzoxazin-3-yl), 5H-,8H-(2,3-benzoxazin-3-yl), 5H-,6H-(2,3-benzoxazin-3-yl), 2,3-benzoxazin-3-yl, 1H-, 4H-,5H-,6H-,7H-,8H-(2,3-benzoxazin-3-yl), 1H-,4H-,7H-,8H-(2,3-benzoxazin-3-yl), 1H-, 4H-,5H-,8H-(2,3-benzoxazin-3-yl), 1H-,4H-,5H-,6H-(2,3-benzoxazin-3-yl), 1H-,4H-(2,3-benzoxazin-3-yl), 4H-,6H-,7H-,8H-(2,3-benzoxazin-3-yl), 4H-,7H-,8H-(2,3-benzoxazin-3-yl), 4H-,5H-,8H-(2,3-benzoxazin-3-yl), 4H-,5H-,6H-(2,3-benzoxazin-3-yl), 4H-,5H-,5aH-,6H-, 7H-,8H-,8aH-(2,3-benzoxazin-3-yl), 4H-,8H-(2,3-benzoxazin-3-yl), 4H-,6H-(2,3-benzoxazin-3-yl), 4H-,5aH-(2,3-benzoxazin-3-yl), 1H-,5H-,6H-,7H-,8H-,8aH-(2,3-benzoxazin-3-yl), 1H-,7H-,8H-,8aH-(2,3-benzoxazin-3-yl), 1H-,5H-,7H-,8H-,8aH-(2,3-benzoxazin-3-yl), 1H-,5H-,6H-,8aH-(2,3-benzoxazin-3-yl), 1H-,5H-,6H-,7H-(2,3-benzoxazin-3-yl), 1H-,8aH-(2,3-benzoxazin-3-yl), 1H-,7H-(2,3-benzoxazin-3-yl), 1H-,5H-(2,3-benzoxazin-3-yl), 1H-,4H-,5H-,5aH-,6H-,7H-,8H-,8aH-(2,3-benzoxazin-3-yl), 1H-,4H-, 6H-,7H-,8H-,8aH-(2,3-benzoxazin-3-yl), 1H-,4H-,5aH-,7H-,8H-,8aH-(2,3-benzoxazin-3-yl), 1H-,4H-,5H-,5aH-,8H-,8aH-(2,3-benzoxazin-3-yl), 1H-,4H-,5H-,5aH-,6H-,8aH-(2,3-benzoxazin-3-yl), 1H-,4H-,5H-,5aH-,6H-,7H-(2,3-benzoxazin-3-yl), 1H-,4H-,6H-,7H-(2,3-benzoxazin-3-yl), 1H-,4H-,5aH-,7H-(2,3-benzoxazin-3-yl), 1H-,4H-,5H-,5aH-(2,3-benzoxazin-3-yl), 1H-,4H-,6H-,8aH-(2,3-benzoxazin-3-yl), 1H-,4H-,8H-,8aH-(2,3-benzoxazin-3-yl),
3,1-benzoxazin-1-yl, 2H-,5H-,6H-,7H-,8H-,8aH-(3,1-benzoxazin-1-yl), 2H-,4H-,5H-, 6H-,7H-,8H-(3,1-benzoxazin-1-yl), 2H-,7H-,8H-,8aH-(3,1-benzoxazin-1-yl), 2H-,5H-,8H-, 8aH-(3,1-benzoxazin-1-yl), 2H-,5H-,6H-,8aH-(3,1-benzoxazin-1-yl), 2H-,5H-,6H-,7H-(3,1-benzoxazin-1-yl), 2H-,5H-(3,1-benzoxazin-1-yl), 2H-,7H-(3,1-benzoxazin-1-yl), 2H-,8 aH-(3,1-benzoxazin-1-yl), 2H-,4H-,7H-,8H-(3,1-benzoxazin-1-yl), 2H-,4H-,5H-,8H-(3,1-benzoxazin-1-yl), 2H-,4H-,5H-,6H-(3,1-benzoxazin-1-yl), 2H-,4H-,6H-,7H-(3,1-benzoxazin-1-yl), 2H-,4H-,5 aH-,8aH-(3,1-benz oxazin-1-yl), 2H-,4H-,5H-,8H-(3,1-benzoxazin-1-yl), 2H-, 4H-,5 aH-,7H-(3,1-benzoxazin-1-yl), 2H-,4H-,6H-,8aH-(3,1-benzoxazin-1-yl), 2H-,4H-,5H-, 6H-(3,1-benzoxazin-1-yl), 2H-,4H-,7H-,8H-(3,1-benzoxazin-1-yl),
1,2-benzisoxazin-2-yl, 5H-,6H-(1,2-benzisoxazin-2-yl), 5H-,8H-(1,2-benzisoxazin-2-yl), 7H-,8H-(1,2-benzisoxazin-2-yl), 5H-,6H-,7H-,8H-(1,2-benzisoxazin-2-yl),
3H-,8aH-(1,2-benzisoxazin-2-yl), 3H-,7H-(1,2-benzisoxazin-2-yl), 3H-,5H-(1,2-benzisoxazin-2-yl), 3H-,7H-,8H-,8aH-(1,2-benzisoxazin-2-yl), 3H-,5H-,8H-,8aH-(1,2-benzisoxazin-2-yl), 3H-,5H-,6H-,8aH-(1,2-benzisoxazin-2-yl), 3H-,5H-,6H-,7H-(1,2-benzisoxazin-2-yl), 3H-,5H-,6H-,7H-,8H-,8aH-(1,2-benzisoxazin-2-yl),
3H-,4H-,7H-,8H-(1,2-benzisoxazin-2-yl), 3H-,4H-,7H-,8H-(1,2-benzisoxazin-2-yl), 3H-,4H-,5H-,8H-(1,2-benzisoxazin-2-yl), 3H-,4H-,5H-,6H-(1,2-benzisoxazin-2-yl), 3H-,4H-, 5H-,6H-,7H-,8H-(1,2-benzisoxazin-2-yl),
5aH-,8aH-(1,2-benzisoxazin-2-yl), 5aH-,7H-(1,2-benzisoxazin-2-yl), 5aH-,7H-,8H-, 8aH-(1,2-benzisoxazin-2-yl), 5H-,5aH-,8H-,8aH-(1,2-benzisoxazin-2-yl), 5H-,5aH-,6H-, 8aH-(1,2-benzisoxazin-2-yl), 5H-,5aH-,6H-,7H-(1,2-benzisoxazin-2-yl), 6H-,8aH-(1,2-benzisoxazin-2-yl), 8H-,8aH-(1,2-benzisoxazin-2-yl), 6H-,7H-(1,2-benzisoxazin-2-yl), 5H-, 5aH-,6H-,7H-,8H-,8aH-(1,2-benzisoxazin-2-yl),
3H-,4H-,5H-,5aH-,6H-,7H-,8H-,8aH-(1,2-benzisoxazin-2-yl), 3H-,4H-,6H-,7H-,8H-, 8aH-(1,2-benzisoxazin-2-yl), 3H-,4H-,5aH-,7H-,8H-,8aH-(1,2-benzisoxazin-2-yl), 3H-,4H-, 5H-,5aH-,8H-,8aH-(1,2-benzisoxazin-2-yl), 3H-,4H-,5H-,5aH-,6H-,8aH-(1,2-benzisoxazin-2-yl), 3H-,4H-,5H-,5aH-,6H-,7H-(1,2-benzisoxazin-2-yl), 3H-,4H-,5H-,6H-,7H-,8H-(1,2-benzisoxazin-2-yl), 3H-,4H-,7H-,8H-(1,2-benzisoxazin-2-yl), 3H-,4H-,5H-,8H-(1,2-benzisoxazin-2-yl), 3H-,4H-,6H-,7H-(1,2-benzisoxazin-2-yl), 3H-,4H-,5H-,6H-(1,2-benzisoxazin-2-yl), 3H-,4H-,5aH-,8aH-(1,2-benzisoxazin-2-yl), 3H-,4H-,8H-,8aH-(1,2-benzisoxazin-2-yl), 3H-,4H-,6H-,8aH-(1,2-benzisoxazin-2-yl), 3H-,4H-,5aH-,7H-(1,2-benzisoxazin-2-yl), 3H-,4H-,5H-,5aH-(1,2-benzisoxazin-2-yl),
purin-1-yl, purin-3-yl, purin-7-yl, purin-9-yl, or hydro compound thereof,
carbazol-9-yl, or hydro compound thereof,
and salt thereof.

31. The compound of claim 1, wherein one or more heterocyclic ring group formed by one or both of R¹ and R² together with the nitrogen to which they are attached and R⁷ and R⁸ together with the nitrogen to which they are attached is independently unsubstituted or substituted with one or more substitutents selected from the group consisting of hydroxyl, halo, bromo, chloro, iodo, fluoro, —OR¹², —NR¹²R¹³, —NR¹²COR¹³, —CONR¹²R¹³, CONHR¹², COOR¹², —OCOR¹², —COR¹², —SR¹², —SO₂R¹², —SO₃R¹², —SO₂NHR¹², —SOR¹², —N₃, —CN, —NC, —SH, —NO₂, —NH₂, —PR₂, —(O)PR¹²R¹³, —PO₃R¹²R¹³, —OPO₃R¹²R¹³, —PO₂, (C₁-C₂₀) alkyl, phenyl, (C₃-C₂₀) cycloalkyl, (C₁-C₂₀) alkoxy, (C₃-C₂₅) heteroaryl, (C₃-C₂₅) heterocyclyl, (C₂-C₂₀) alkenyl, (C₄-C₂₀) cycloalkenyl, (C₂-C₂₀) alkynyl, (C₆-C₂₀) cycloalkynyl, (C₅-C₂₅) aryl, perhalo (C₁-C₂₀) alkyl, salt thereof, and a combination thereof.

32. The compound of claim 1, wherein one or more of R¹, R², R³, R⁴, R⁵, R⁶, R⁷, R⁸, R⁹, R¹⁰, and R¹¹ is independently a (C₃-C₂₅) heterocyclyl selected from the group consisting of furan, thiophene, pyrrole, isopyrrole, pyrazole, isodiazole, triazole, dithole, oxathole, isoxazole, thiazole, isothiazole, oxadiazole, dioxazole, oxathiazole, oxathiole, pyran, pyridine, pyridazine, pyrimidine, pyrazine, piperazine, morpholino, triazine, oxazine, isoxazine, oxathiazine, azepine, oxepin, thiepin, diazepine, benzofuran, isobenzofuran, thionaphthene, isothionaphthene, indole, isbenzazole, pyrindine, pyrano[3,4-b]pyrrole, isoindazole, indoxazine, benzoxazole, anthranil, benzopyran, quinoline, isoquinoline, cinnoline, quinazoline, nahthyridine, pyrido[3,4-b]pyridine, pyrido[3,2-b]pyridine, pyrido[4,3-b-]pyridine, benzoxazine, carbazole, xanthene, acridine, purine, hydro compound thereof, and salt thereof.

33. A composition, comprising at least one pharmaceutically acceptable carrier and at least one compound having the following formula: 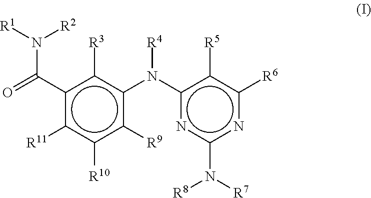
or salt thereof;
wherein R¹ and R² are each independently selected from the group consisting of hydrogen, hydroxyl, halo, bromo, chloro, iodo, fluoro, —NR¹²COR¹³, —CONR¹²R¹³, CONHR¹², —COOR¹², —OCOR¹², —COR¹², —SR¹², —SO₂NHR¹², —SO₃R¹², —SO₂NHR¹², —SOR¹², —N₃, —CN, —NC, —SH, —NO₂, —NH₂, —PR₂, —(O)PR¹²R¹³, —PO₃R¹²R¹³, —OPO₃R¹²R¹³, —PO₂, (C₁-C₂₀) alkyl, phenyl, (C₃-C₂₀) cycloalkyl, (C₁-C₂₀) alkoxy, (C₃-C₂₅) heteroaryl, (C₃-C₂₅) heterocyclyl, (C₂-C₂₀) alkenyl, (C₄-C₂₀) cycloalkenyl, (C₂-C₂₀) alkynyl, (C₆-C₂₀) cycloalkynyl, (C₅-C₂₅) aryl, perhalo (C₁-C₂₀) alkyl, and salt thereof;
or wherein R¹ and R² together with the nitrogen to which they are attached form a heterocyclic ring group or salt thereof;
wherein R³, R⁹, and R¹¹ are each independently selected from the group consisting of hydrogen, hydroxyl, halo, bromo, chloro, iodo, fluoro, halo, bromo, chloro, iodo, fluoro, —OR¹², —NR¹²R¹³, —NR¹²COR¹³, —CONR¹²R¹³, —CONHR¹², —COOR¹², —OCOR¹², —COR¹², —SR¹², —SO₂R¹², —SO₃R¹², —SO₂NHR¹², —SOR¹², —N₃, —CN, —NC, —SH, NO₂, —NH₂, —PR₂, —(O)PR¹²R¹³, —PO₃R¹²R¹³, —OPO₃R¹²R¹³, —PO₂, (C₁-C₂₀) alkyl, phenyl, (C₃-C₂₀) cycloalkyl, (C₁-C₂₀) alkoxy, (C₃-C₂₅) heteroaryl, (C₃-C₂₅) heterocyclyl, (C₂-C₂₀) alkenyl, (C₄-C₂₀) cycloalkenyl, (C₂-C₂₀) alkynyl, (C₆-C₂₀) cycloalkynyl, (C₅-C₂₅) aryl, perhalo (C₁-C₂₀) alkyl, and salt thereof;
wherein R⁴ is hydrogen, hydroxyl, halo, bromo, chloro, iodo, fluoro, —OR¹², —NR¹²R¹³, —NR¹²COR¹³, —CONR¹²R¹³, —CONHR¹², —COOR¹², —OCOR¹², —COR¹², —SR¹², —₂R¹², —SO₃R¹², —SO₂NHR¹², —SOR¹², —CN, —NC, —SH, —NO₂, —NH₂, PR₂,—(O)PR¹²R¹³, —PO₃R¹²R¹³, —OPO₃R¹²R¹³, —PO₂, (C₁-C₂₀) alkyl, phenyl, (C₃-C₂₀) cycloalkyl, (C₁-C₂₀) alkoxy, (C₃-C₂₅) heteroaryl, (C₃-C₂₅) heterocyclyl, (C₂-C₂₀) alkenyl, (C₄-C₂₀) cycloalkenyl, (C₂-C₂₀) alkynyl, (C₆-C₂₀) cycloalkynyl, (C₅-C₂₅) aryl, perhalo (C₁-C₂₀) alkyl, or salt thereof
wherein R⁵ and R⁶ are each independently selected from the group consisting of hydrogen, hydroxyl, halo, bromo, chloro, iodo, fluoro, —OR¹², —NR¹²R¹³, —NR¹²COR¹³, —CONR¹²R¹³, —CONHR¹², —COOR¹², —OCOR¹², —COR¹², —SR¹², —SO₂R¹², —SO₃R¹², —SO₂NHR¹², —SOR¹², —N₃, —CN, —NC, —SH, —NO₂, —NH₂, —PR₂, —(O)PR¹²R¹³, —PO₃R¹²R¹³, —OPO₃R¹²R¹³, —PO₂, (C₁-C₂₀) alkyl, phenyl, (C₃-C₂₀) cycloalkyl, (C₁-C₂₀) alkoxy, (C₃-C₂₅) heteroaryl, (C₃-C₂₅) heterocyclyl, (C₂-C₂₀) alkenyl, (C₄-C₂₀) cycloalkenyl, (C₂-C₂₀) alkynyl, (C₆-C₂₀) cycloalkynyl, (C₅-C₂₅) aryl, perhalo (C₁-C₂₀) alkyl, and salt thereof;
or wherein R⁵ and R⁶ together with the carbons to which they are attached form a (C₅-C₆) aryl structure, a (C₃-C₆) heteroaryl structure, a (C₃-C₆) heterocycloalkenyl structure, a (C₄-C₆) cycloalkenyl structure, or salt thereof;
wherein R⁷ and R⁸ are each independently selected from the group consisting of hydrogen, hydroxyl, halo, bromo, chloro, iodo, fluoro, —C(NH)NH₂, —C(NH)NHC(NH)NH₂, —OR¹², —NR¹²R¹³, —NR¹²COR¹³, —CONR¹²R¹³, CONHR¹², —COOR¹², —OCOR¹², —COR¹², —SR¹², —SO₂R¹², —SO₃R¹², —SO₂NHR¹², —SOR¹², —CN, —NC, —SH, —NO₂, —NH₂, —PR₂, —(O)PR¹²R¹³, —PO₃R¹²R¹³, —OPO₃R¹²R¹³, PO₂, (C₁-C₂₀) alkyl, phenyl, (C₃-C₂₀) cycloalkyl, (C₁-C₂₀) alkoxy, (C₃-C₂₅) heteroaryl, (C₃-C₂₅) heterocyclyl, (C₂-C₂₀) alkenyl, (C₄-C₂₀) cycloalkenyl, (C₂-C₂₀) alkynyl, (C₆-C₂₀) cycloalkynyl, (C₅-C₂₅) aryl, perhalo (C₁-C₂₀) alkyl, and salt thereof;
or wherein R⁷ and R⁸ together with the nitrogen to which they are attached form a heterocyclic ring group or salt thereof;
wherein R¹⁰ is hydrogen, hydroxyl, halo, bromo, chloro, iodo, fluoro, —OR¹⁴, —NR¹⁴R¹⁵, —NR¹⁴COR¹⁵, —CONR¹⁴R¹⁵, —CONR¹⁴, —COOR¹⁴, —OCOR¹⁴, —COR¹⁴, —SR¹⁴, —SO₂R¹⁴, —SO₃R¹⁴, —SO₂NR¹⁴, —SOR¹⁴, —N₃, —CN, —NC, —SH, —NO₂, —NH₂, —PR₂, —(O)PR¹⁴R¹⁵, —PO₃R¹⁴R¹⁵, OPO₃R¹⁴R¹⁵, —PO₂, (C₁-C₂₀) alkyl, phenyl, (C₃-C₂₀) cycloalkyl, (C₁-C₂₀) alkoxy, (C₃-C₂₅) heteroaryl, (C₃-C₂₅) heterocyclyl, (C₂-C₂₀) alkenyl, (C₄-C₂₀) cycloalkenyl, (C₂-C₂₀) alkynyl, (C₆-C₂₀) cycloalkynyl, (C₅-C₂₅) aryl, perhalo (C₁-C₂₀) alkyl, or salt thereof;
wherein R¹² and R¹³ are each independently selected from the group consisting of (C₁-C₂₀) alkyl, phenyl, (C₃-C₂₀) cycloalkyl, (C₁-C₂₀) alkoxy, (C₃-C₂₅) heteroaryl, (C₃-C₂₅) heterocyclyl, (C₂-C₂₀) alkenyl, (C₄-C₂₀) cycloalkenyl, (C₂-C₂₀) alkynyl, (C₆-C₂₀) cycloalkynyl, (C₅-C₂₅) aryl, perhalo (C₁-C₂₀) alkyl, and salt thereof;
wherein R¹⁴ and R¹⁵ are each independently selected from the group consisting of hydrogen, hydroxyl, halo, bromo, chloro, iodo, fluoro, —OR¹², —NR¹²R¹³, —NR¹²COR¹³, —CONR¹²R¹³, —CONHR¹², —COOR¹², —OCOR¹², —COR¹², —SR¹², —SO₂R¹², —SO₃R¹², —SO₂NHR¹², —SOR¹², —N₃, —CN, —NC, —SH, —NO₂, —NH₂, —PR₂, —(O)PR¹²R¹³, —PO₃R¹²R¹³, —OPO₃R¹²R¹³, —PO₂, (C₁-C₂₀) alkyl, phenyl, (C₃-C₂₀) cycloalkyl, (C₁-C₂₀) alkoxy, (C₃-C₂₅) heteroaryl, (C₃-C₂₅) heterocyclyl, (C₂-C₂₀) alkenyl, (C₄-C₂₀) cycloalkenyl, (C₂-C₂₀) alkynyl, (C₆-C₂₀) cycloalkynyl, (C₅-C₂₅) aryl, perhalo (C₁-C₂₀) alkyl, and salt thereof;
and wherein each of said (C₁-C₂₀) alkyl, phenyl, (C₃-C₂₀) cycloalkyl, (C₁-C₂₀) alkoxy, (C₃-C₂₅) heteroaryl, (C₃-C₂₅) heterocyclyl, (C₂-C₂₀) alkenyl, (C₄-C₂₀) cycloalkenyl, (C₂-C₂₀) alkynyl, (C₆-C₂₀) cycloalkynyl, (C₅-C₂₅) aryl, perhalo (C₁-C₂₀) alkyl, (C₃-C₆) cycloalkyl structure, (C₅-C₆) aryl structure, (C₃-C₆) heteroaryl structure, (C₃-C₆) heterocyclyl structure, (C₃-C₆) heterocycloalkenyl structure, (C₄-C₆) cycloalkenyl structure, heterocyclic ring group, or salt thereof may be independently unsubstituted or substituted with one or more substituents selected from the group consisting of hydroxyl, halo, bromo, chloro, iodo, fluoro, —OR¹², —NR¹²R¹³, —NR¹²COR¹³, —CONR¹²R¹³, —CONHR¹², —COOR¹², —OCOR¹², —COR¹², —SR¹², —SO₂R¹², —SO₃R¹², —SO₂NHR¹², —SOR¹², —N₃, —CN, —NC, —SH, —NO₂, —NH₂, —PR₂, —(O)PR¹²R¹³, —PO₃R¹²R¹³, —OPO₃R¹²R¹³, —PO₂, (C₁-C₂₀) alkyl, phenyl, (C₃-C₂₀) cycloalkyl, (C₁-C₂₀) alkoxy, (C₃-C₂₅) heteroaryl, (C₃-C₂₅) heterocyclyl, (C₂-C₂₀) alkenyl, (C₄-C₂₀) cycloalkenyl, (C₂-C₂₀) alkynyl, (C₆-C₂₀) cycloalkynyl, (C₅-C₂₅) aryl, perhalo (C₁-C₂₀) alkyl, salt thereof, and a combination thereof.

34. The composition of claim 33, wherein when R³, R¹⁰, and R¹¹ are hydrogen, and R⁹ is —CH₃, then R⁵ is not —CN.

35. A composition, comprising:
at least one inhibitor selected from the group consisting of Nucleoside analog Reverse Transcriptase inhibitor (NRTi), —Non-Nucleoside analog Reverse Transcriptase inhibitor (NNRTi), Protease inhibitor (Pi), Cell Entry inhibitor (Ci), and a combination thereof; and
at least one compound having the following formula: 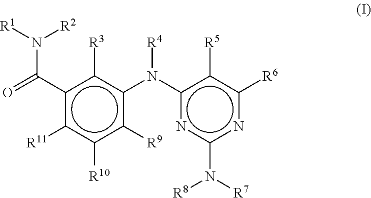
or salt thereof;
wherein R¹ and R² are each independently selected from the group consisting of hydrogen, hydroxyl, halo, bromo, chloro, iodo, fluoro, —NR¹²COR¹³, —CONR¹²R¹³, —CONHR¹², —COOR¹², —OCOR¹², —COR¹², —SR¹², —SO₂NHR¹², —SO₃R¹², —SO₂NHR¹², —SOR¹², —N₃, —CN, —NC, —SH, —NO₂, —NH₂, —PR₂, —(O)PR¹²R¹³, —PO₃R¹²R¹³, —OPO₃R¹²R¹³, —PO₂, (C₁-C₂₀) alkyl, phenyl, (C₃-C₂₀) cycloalkyl, (C₁-C₂₀) alkoxy, (C₃-C₂₅) heteroaryl, (C₃-C₂₅) heterocyclyl, (C₂-C₂₀) alkenyl, (C₄-C₂₀) cycloalkenyl, (C₂-C₂₀) alkynyl, (C₆-C₂₀) cycloalkynyl, (C₅-C₂₅) aryl, perhalo (C₁-C₂₀) alkyl, and salt thereof;
or wherein R¹ and R² together with the nitrogen to which they are attached form a heterocyclic ring group or salt thereof;
wherein R³, R⁹, and R¹¹ are each independently selected from the group consisting of hydrogen, hydroxyl, halo, bromo, chloro, iodo, fluoro, halo, bromo, chloro, iodo, fluoro, —OR¹², —NR¹²R¹³, —NR¹²COR¹³, —CONR¹²R¹³, —CONHR¹², —COOR¹², —OCOR¹², —COR¹², —SR¹², —SO₂R¹², —SO₃R¹², —SO₂NHR¹², —SOR¹², —N₃, —CN, —NC, —SH, —NO₂, —NH₂, —PR₂, —(O)PR¹²R¹³, —PO₃R¹²R¹³, —OPO₃R¹²R¹³, —PO₂, (C₁-C₂₀) alkyl, phenyl, (C₃-C₂₀) cycloalkyl, (C₁-C₂₀) alkoxy, (C₃-C₂₅) heteroaryl, (C₃-C₂₅) heterocyclyl, (C₂-C₂₀) alkenyl, (C₄-C₂₀) cycloalkenyl, (C₂-C₂₀) alkynyl, (C₆-C₂₀) cycloalkynyl, (C₅-C₂₅) aryl, perhalo (C₁-C₂₀) alkyl, and salt thereof;
wherein R⁴ is hydrogen, hydroxyl, halo, bromo, chloro, iodo, fluoro, —OR¹², —NR¹²R¹³, —NR¹²COR¹³, —CONR¹²R¹³, —CONHR¹², —COOR¹², —OCOR¹², —COR¹², —SR¹², —SO₂R¹², —SO₃R¹², —SO₂NHR¹², —SOR¹², —CN, —NC, —SH, —NO₂, —NH₂, —PR₂,—(O)PR¹²R¹³, —PO₃R¹²R¹³, —OPO₃R¹²R¹³, —PO₂, (C₁-C₂₀) alkyl, phenyl, (C₃-C₂₀) cycloalkyl, (C₁-C₂₀) alkoxy, (C₃-C₂₅) heteroaryl, (C₃-C₂₅) heterocyclyl, (C₂-C₂₀) alkenyl, (C₄-C₂₀) cycloalkenyl, (C₂-C₂₀) alkynyl, (C₆-C₂₀) cycloalkynyl, (C₅-C₂₅) aryl, perhalo (C₁-C₂₀) alkyl, or salt thereof
wherein R⁵ and R⁶ are each independently selected from the group consisting of hydrogen, hydroxyl, halo, bromo, chloro, iodo, fluoro, —OR¹², —NR¹²R¹³, —NR¹²COR¹³, CONR¹²R¹³, —CONHR¹², —COOR¹², —OCOR¹², —COR¹², —SR¹², —SO₂R¹², —SO₃R¹², —SO₂NHR¹², —SOR¹², —N₃, —CN, —NC, —SH, —NO₂, —NH₂, —PR₂, —(O)PR¹²R¹³, —PO₃R¹²R¹³, —OPO₃R¹²R¹³, —PO₂, (C₁-C₂₀) alkyl, phenyl, (C₃-C₂₀) cycloalkyl, (C₁-C₂₀) alkoxy, (C₃-C₂₅) heteroaryl, (C₃-C₂₅) heterocyclyl, (C₂-C₂₀) alkenyl, (C₄-C₂₀) cycloalkenyl, (C₂-C₂₀) alkynyl, (C₆-C₂₀) cycloalkynyl, (C₅-C₂₅) aryl, perhalo (C₁-C₂₀) alkyl, and salt thereof;
or wherein R⁵ and R⁶ together with the carbons to which they are attached form a (C₅-C₆) aryl structure, a (C₃-C₆) heteroaryl structure, a (C₃-C₆) heterocycloalkenyl structure, a (C₄-C₆) cycloalkenyl structure, or salt thereof;
wherein R⁷ and R⁸ are each independently selected from the group consisting of hydrogen, hydroxyl, halo, bromo, chloro, iodo, fluoro, —C(NH)NH₂, C(NH)NHC(NH)NH₂, —OR¹², —NR¹²R¹³, —NR¹²COR¹³, —CONR¹²R¹³, —CONHR¹², —COOR¹², —OCOR¹², —COR¹², —SR¹², —SO₂R¹², —SO₃R¹², —SO₂NHR¹², —SOR¹², —CN, —NC, —SH, —NO₂, —NH₂, —PR₂, —(O)PR¹²R¹³, —PO₃R¹²R¹³, OPO₃R¹²R¹³, —PO₂, (C₁-C₂₀) alkyl, phenyl, (C₃-C₂₀) cycloalkyl, (C₁-C₂₀) alkoxy, (C₃-C₂₅) heteroaryl, (C₃-C₂₅) heterocyclyl, (C₂-C₂₀) alkenyl, (C₄-C₂₀) cycloalkenyl, (C₂-C₂₀) alkynyl, (C₆-C₂₀) cycloalkynyl, (C₅-C₂₅) aryl, perhalo (C₁-C₂₀) alkyl, and salt thereof;
or wherein R⁷ and R⁸ together with the nitrogen to which they are attached form a heterocyclic ring group or salt thereof;
wherein R¹⁰ is hydrogen, hydroxyl, halo, bromo, chloro, iodo, fluoro, —OR¹⁴, NR¹⁴R¹⁵, —NR¹⁴COR¹⁵, —CONR¹⁴R¹⁵, —CONR¹⁴, —COOR¹⁴, —OCOR¹⁴, —COR¹⁴, SR¹⁴, —SO₂R¹⁴, —SO₃R¹⁴, —SO₂NR¹⁴, —SOR¹⁴, —N₃, —CN, —NC, —SH, —NO₂, NH₂, —PR₂, —(O)PR¹⁴R¹⁵, —PO₃R¹⁴R¹⁵, —OPO₃R¹⁴R¹⁵, —PO₂, (C₁-C₂₀) alkyl, phenyl, (C₃-C₂₀) cycloalkyl, (C₁-C₂₀) alkoxy, (C₃-C₂₅) heteroaryl, (C₃-C₂₅) heterocyclyl, (C₂-C₂₀) alkenyl, (C₄-C₂₀) cycloalkenyl, (C₂-C₂₀) alkynyl, (C₆-C₂₀) cycloalkynyl, (C₅-C₂₅) aryl, perhalo (C₁-C₂₀) alkyl, or salt thereof;
wherein R¹² and R¹³ are each independently selected from the group consisting of (C₁-C₂₀) alkyl, phenyl, (C₃-C₂₀) cycloalkyl, (C₁-C₂₀) alkoxy, (C₃-C₂₅) heteroaryl, (C₃-C₂₅) heterocyclyl, (C₂-C₂₀) alkenyl, (C₄-C₂₀) cycloalkenyl, (C₂-C₂₀) alkynyl, (C₆-C₂₀) cycloalkynyl, (C₅-C₂₅) aryl, perhalo (C₁-C₂₀) alkyl, and salt thereof;
wherein R¹⁴ and R¹⁵ are each independently selected from the group consisting of hydrogen, hydroxyl, halo, bromo, chloro, iodo, fluoro, —OR¹², —NR¹²R¹³, —NR¹²COR¹³, CONR¹²R¹³, —CONHR¹², —COOR¹², —OCOR¹², —COR¹², —SR¹², —SO₂R¹², —SO₃R¹², —SO₂NHR¹², —SOR¹², —N₃, —CN, —NC, —SH, —NO₂, —NH₂, —PR₂, —(O)PR¹²R¹³, —PO₃R¹²R¹³, —OPO₃R¹²R¹³, —PO₂, (C₁-C₂₀) alkyl, phenyl, (C₃-C₂₀) cycloalkyl, (C₁-C₂₀) alkoxy, (C₃-C₂₅) heteroaryl, (C₃-C₂₅) heterocyclyl, (C₂-C₂₀) alkenyl, (C₄-C₂₀) cycloalkenyl, (C₂-C₂₀) alkynyl, (C₆-C₂₀) cycloalkynyl, (C₅-C₂₅) aryl, perhalo (C₁-C₂₀) alkyl, and salt thereof;
and wherein each of said (C₁-C₂₀) alkyl, phenyl, (C₃-C₂₀) cycloalkyl, (C₁-C₂₀) alkoxy, (C₃-C₂₅) heteroaryl, (C₃-C₂₅) heterocyclyl, (C₂-C₂₀) alkenyl, (C₄-C₂₀) cycloalkenyl, (C₂-C₂₀) alkynyl, (C₆-C₂₀) cycloalkynyl, (C₅-C₂₅) aryl, perhalo (C₁-C₂₀) alkyl, (C₃-C₆) cycloalkyl structure, (C₅-C₆) aryl structure, (C₃-C₆) heteroaryl structure, (C₃-C₆) heterocyclyl structure, (C₃-C₆) heterocycloalkenyl structure, (C₄-C₆) cycloalkenyl structure, heterocyclic ring group, or salt thereof may be independently unsubstituted or substituted with one or more substituents selected from the group consisting of hydroxyl, halo, bromo, chloro, iodo, fluoro, —OR¹², —NR¹²R¹³, —NR¹²COR¹³, —CONR¹²R¹³, —CONHR¹², —COOR¹², —OCOR¹², —COR¹², —SR¹², —SO₂R¹², —SO₃R¹², —SO₂NHR¹², —SOR¹², —N₃, —CN, —NC, —SH, —NO₂, —NH₂, —PR₂, —(O)PR¹²R¹³, —PO₃R¹²R¹³, —OPO₃R¹²R¹³, —PO₂, (C₁-C₂₀) alkyl, phenyl, (C₃-C₂₀) cycloalkyl, (C₁-C₂₀) alkoxy, (C₃-C₂₅) heteroaryl, (C₃-C₂₅) heterocyclyl, (C₂-C₂₀) alkenyl, (C₄-C₂₀) cycloalkenyl, (C₂-C₂₀) alkynyl, (C₆-C₂₀) cycloalkynyl, (C₅-C₂₅) aryl, perhalo (C₁-C₂₀) alkyl, salt thereof, and a combination thereof.

36. The composition of claim 35, wherein when R³, R¹⁰, and R¹¹ are hydrogen, and R⁹ is —CH₃, then R⁵ is not —CN.

37. The composition of claim 35, wherein the inhibitor is a Nucleoside analog Reverse Transcriptase inhibitor (NRTi).

38. The composition of claim 35, wherein the inhibitor is a Nucleoside analog Reverse Transcriptase inhibitor (NRTi) selected from the group consisting of AZT, 3TC, FTC, ABC, ddC, ZDV, TDF, ddI, DFC, DAPD, alovidine, elvucitabine, D4T, RCV, Beta-L-Fd4C, and a combination thereof.

39. The composition of claim 35, wherein the inhibitor is a Non-Nucleoside analog Reverse Transcriptase inhibitor (NNRTi).

40. The composition of claim 35, wherein the inhibitor is a Non-Nucleoside analog Reverse Transcriptase inhibitor (NNRTi) selected from the group consisting of DLV, EFV, NVP, calanolide A, etravirine, TMC-278, BMS-561390, capravirine, and a combination thereof.

41. The composition of claim 35, wherein the inhibitor is a Protease inhibitor (Pi).

42. The composition of claim 35, wherein the inhibitor is a Protease inhibitor (Pi) selected from the group consisting of APV, TPV, IDV, SQV, LPV, FPV, RTV, ATZ, —NFV, brecanavir, darunavir, PPL-100, L-756423, RO033-4649, and a combination thereof.

43. The composition of claim 35, wherein the inhibitor is a Cell Entry inhibitor (Ci).

44. The composition of claim 35, wherein the inhibitor is a Cell Entry inhibitor (Ci) selected from the group consisting of Fuzeon, ENF, aplaviroc, maraviroc, vicriviroc, T-1249, PRO-542, TNX-355, SCH-C, PRO-140, SP-01A, SP-10, TNX-355, and a combination thereof.

45. The composition of claim 35, further comprising a pharmaceutically acceptable carrier.

46. A method for treating a known or suspected malady, in an animal, selected from the group consisting of HIV infection, hepatitis C, hepatitis B, hepatitis delta, influenza, herpes, adenovirus, papillomavirus, parvovirus, measles, bird flu, and a combination thereof, comprising administering, to the animal,
at least one compound having the following formula: 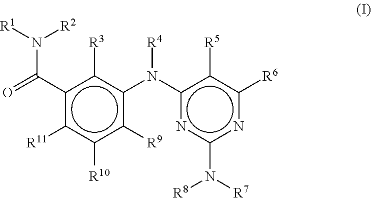
or salt thereof;
wherein R¹ and R² are each independently selected from the group consisting of hydrogen, hydroxyl, halo, bromo, chloro, iodo, fluoro, —NR¹²COR¹³, —CONR¹²R¹³, —CONHR¹², —COOR¹², —OCOR¹², —COR¹², —SR¹², —SO₂R¹², —SO₃R¹², SO₂NHR¹², —SOR¹², —N₃, —CN, —NC, —SH, —NO₂, —NH₂, —PR₂, —(O)PR¹²R¹³, —PO₃R¹²R¹³, —OPO₃R¹²R¹³, —PO₂, (C₁-C₂₀) alkyl, phenyl, (C₃-C₂₀) cycloalkyl, (C₁-C₂₀) alkoxy, (C₃-C₂₅) heteroaryl, (C₃-C₂₅) heterocyclyl, (C₂-C₂₀) alkenyl, (C₄-C₂₀) cycloalkenyl, (C₂-C₂₀) alkynyl, (C₆-C₂₀) cycloalkynyl, (C₅-C₂₅) aryl, perhalo (C₁-C₂₀) alkyl, and salt thereof;
or wherein R¹ and R² together with the nitrogen to which they are attached form a heterocyclic ring group or salt thereof;
wherein R³, R⁹, and R¹¹ are each independently selected from the group consisting of hydrogen, hydroxyl, halo, bromo, chloro, iodo, fluoro, halo, bromo, chloro, iodo, fluoro, —OR¹², —NR¹²R¹³, —NR¹²COR¹³, —CONR¹²R¹³, —CONHR¹², —COOR¹², —OCOR¹², —COR¹², —SR¹², —SO₂R¹², —SO₃R¹², —SO₂NHR¹², —SOR¹², —N₃, —CN, —NC, —SH, NO₂, —NH₂, —PR₂, —(O)PR¹²R¹³, —PO₃R¹²R¹³, OPO₃R¹²R¹³, —PO₂, (C₁-C₂₀) alkyl, phenyl, (C₃-C₂₀) cycloalkyl, (C₁-C₂₀) alkoxy, (C₃-C₂₅) heteroaryl, (C₃-C₂₅) heterocyclyl, (C₂-C₂₀) alkenyl, (C₄-C₂₀) cycloalkenyl, (C₂-C₂₀) alkynyl, (C₆-C₂₀) cycloalkynyl, (C₅-C₂₅) aryl, perhalo (C₁-C₂₀) alkyl, and salt thereof;
wherein R⁴ is hydrogen, hydroxyl, halo, bromo, chloro, iodo, fluoro, —OR¹², —NR¹²R¹³, —NR¹²COR¹³, —CONR¹²R¹³, —CONHR¹², —COOR¹², —OCOR¹², —COR¹², —SR¹², —SO₂R¹², —SO₃R¹², —SO₂NHR¹², —SOR¹², —CN, —NC, —SH, —NO₂, —NH₂, —PR₂, —(O)PR¹²R¹³, —PO₃R¹²R¹³, —OPO₃R¹²R¹³, —PO₂, (C₁-C₂₀) alkyl, phenyl, (C₃-C₂₀) cycloalkyl, (C₁-C₂₀) alkoxy, (C₃-C₂₅) heteroaryl, (C₃-C₂₅) heterocyclyl, (C₂-C₂₀) alkenyl, (C₄-C₂₀) cycloalkenyl, (C₂-C₂₀) alkynyl, (C₆-C₂₀) cycloalkynyl, (C₅-C₂₅) aryl, perhalo (C₁-C₂₀) alkyl, or salt thereof
wherein R⁵ and R⁶ are each independently selected from the group consisting of hydrogen, hydroxyl, halo, bromo, chloro, iodo, fluoro, —OR¹², —NR¹²R¹³, —NR¹²COR¹³, —CONR¹²R¹³, —CONHR¹², —COOR¹², —OCOR¹², —COR¹², —SR¹², —SO₂R¹², —SO₃R¹², —SO₂NHR¹², —SOR¹², —N₃, —CN, —NC, —SH, —NO₂, —NH₂, —PR₂, —(O)PR¹²R¹³, —PO₃R¹²R¹³, —OPO₃R¹²R¹³, —PO₂, (C₁-C₂₀) alkyl, phenyl, (C₃-C₂₀) cycloalkyl, (C₁-C₂₀) alkoxy, (C₃-C₂₅) heteroaryl, (C₃-C₂₅) heterocyclyl, (C₂-C₂₀) alkenyl, (C₄-C₂₀) cycloalkenyl, (C₂-C₂₀) alkynyl, (C₆-C₂₀) cycloalkynyl, (C₅-C₂₅), perhalo (C₁-C₂₀) alkyl, and salt thereof;
or wherein R⁵ and R⁶ together with the carbons to which they are attached form a (C₅-C₆) aryl structure, a (C₃-C₆) heteroaryl structure, a (C₃-C₆) heterocycloalkenyl structure, a (C₄-C₆) cycloalkenyl structure, or salt thereof;
wherein R⁷ and R⁸ are each independently selected from the group consisting of hydrogen, hydroxyl, halo, bromo, chloro, iodo, fluoro, —C(NH)NH₂, —C(NH)NHC(NH)NH₂, —OR¹², —NR¹²R¹³, —NR¹²COR¹³, —CONR¹²R¹³, —CONHR¹², —COOR¹², —OCOR¹², —COR¹², —SR¹², —SO₂R¹², —SO₃R¹², —SO₂NHR¹², —SOR¹², CN, —NC, —SH, —NO₂, —NH₂, —PR₂, —(O)PR¹²R¹³, —PO₃R¹²R¹³, —OPO₃R¹²R¹³, —PO₂, (C₁-C₂₀) alkyl, phenyl, (C₃-C₂₀) cycloalkyl, (C₁-C₂₀) alkoxy, (C₃-C₂₅) heteroaryl, (C₃-C₂₅) heterocyclyl, (C₂-C₂₀) alkenyl, (C₄-C₂₀) cycloalkenyl, (C₂-C₂₀) alkynyl, (C₆-C₂₀) cycloalkynyl, (C₅-C₂₅) aryl, perhalo (C₁-C₂₀) alkyl, and salt thereof;
or wherein R⁷ and R⁸ together with the nitrogen to which they are attached form a heterocyclic ring group or salt thereof;
wherein R¹⁰ is hydrogen, hydroxyl, halo, bromo, chloro, iodo, fluoro, —OR¹⁴, —NR¹⁴R¹⁵, —NR¹⁴COR¹⁵, —CONR¹⁴R¹⁵, —CONR¹⁴, —COOR¹⁴, —OCOR¹⁴, —COR¹⁴, —SR¹⁴, —SO₂R¹⁴, —SO₃R¹⁴, —SO₂NR¹⁴, —SOR¹⁴, —N₃, —CN, —NC, —SH, —NO₂, —NH₂, —PR₂, —(O)PR¹⁴R¹⁵, —PO₃R¹⁴R¹⁵, —OPO₃R¹⁴R¹⁵, —PO₂, (C₁-C₂₀) alkyl, phenyl, (C₃-C₂₀) cycloalkyl, (C₁-C₂₀) alkoxy, (C₃-C₂₅) heteroaryl, (C₃-C₂₅) heterocyclyl, (C₂-C₂₀) alkenyl, (C₄-C₂₀) cycloalkenyl, (C₂-C₂₀) alkynyl, (C₆-C₂₀) cycloalkynyl, (C₅-C₂₅) aryl, perhalo (C₁-C₂₀) alkyl, or salt thereof;
wherein R¹² and R¹³ are each independently selected from the group consisting of (C₁-C₂₀) alkyl, phenyl, (C₃-C₂₀) cycloalkyl, (C₁-C₂₀) alkoxy, (C₃-C₂₅) heteroaryl, (C₃-C₂₅) heterocyclyl, (C₂-C₂₀) alkenyl, (C₄-C₂₀) cycloalkenyl, (C₂-C₂₀) alkynyl, (C₆-C₂₀) cycloalkynyl, (C₅-C₂₅) aryl, perhalo (C₁-C₂₀) alkyl, and salt thereof;
wherein R¹⁴ and R¹⁵ are each independently selected from the group consisting of hydrogen, hydroxyl, halo, bromo, chloro, iodo, fluoro, —OR¹², —NR¹²R¹³, —NR¹²COR¹³, —CONR¹²R¹³, —CONHR¹², —COOR¹², —OCOR¹², —COR¹², —SR¹², —SO₂R¹², —SO₃R¹², —SO₂NHR¹², —SOR¹², —N₃, —CN, —NC, —SH, —NO₂, —NH₂, —PR₂, —(O)PR¹²R¹³, —PO₃R¹²R¹³, —OPO₃R¹²R¹³, —PO₂, (C₁-C₂₀) alkyl, phenyl, (C₃-C₂₀) cycloalkyl, (C₁-C₂₀) alkoxy, (C₃-C₂₅) heteroaryl, (C₃-C₂₅) heterocyclyl, (C₂-C₂₀) alkenyl, (C₄-C₂₀) cycloalkenyl, (C₂-C₂₀) alkynyl, (C₆-C₂₀) cycloalkynyl, (C₅-C₂₅) aryl, perhalo (C₁-C₂₀) alkyl, and salt thereof;
and wherein each of said (C₁-C₂₀) alkyl, phenyl, (C₃-C₂₀) cycloalkyl, (C₁-C₂₀) alkoxy, (C₃-C₂₅) heteroaryl, (C₃-C₂₅) heterocyclyl, (C₂-C₂₀) alkenyl, (C₄-C₂₀) cycloalkenyl, (C₂-C₂₀) alkynyl, (C₆-C₂₀) cycloalkynyl, (C₅-C₂₅) aryl, perhalo (C₁-C₂₀) alkyl, (C₃-C₆) cycloalkyl structure, (C₅-C₆) aryl structure, (C₃-C₆) heteroaryl structure, (C₃-C₆) heterocyclyl structure, (C₃-C₆) heterocycloalkenyl structure, (C₄-C₆) cycloalkenyl structure, heterocyclic ring group, or salt thereof may be independently unsubstituted or substituted with one or more substituents selected from the group consisting of hydroxyl, halo, bromo, chloro, iodo, fluoro, —OR¹², —NR¹²R¹³, —NR¹²COR¹³, —CONR¹²R¹³, —CONHR¹², —COOR¹², —OCOR¹², —COR¹², —SR¹², —SO₂R¹², —SO₃R¹², —SO₂NHR¹², —SOR¹², —N₃, —CN, —NC, —SH, —NO₂, —NH₂, —PR₂,—(O)PR¹²R¹³, —PO₃R¹²R¹³, —OPO₃R¹²R¹³, —PO₂, (C₁-C₂₀) alkyl, phenyl, (C₃-C₂₀) cycloalkyl, (C₁-C₂₀) alkoxy, (C₃-C₂₅) heteroaryl, (C₃-C₂₅) heterocyclyl, (C₂-C₂₀) alkenyl, (C₄-C₂₀) cycloalkenyl, (C₂-C₂₀) alkynyl, (C₆-C₂₀) cycloalkynyl, (C₅-C₂₅) aryl, perhalo (C₁-C₂₀) alkyl, salt thereof, and a combination thereof.

47. The method of claim 46, wherein when R³, R¹⁰, and R¹¹ are hydrogen, and R⁹ is —CH₃, then R⁵ is not —CN.

48. The method of claim 46, further comprising administering at least one inhibitor selected from the group consisting of Nucleoside analog Reverse Transcriptase inhibitor (NRTi), —Non-Nucleoside analog Reverse Transcriptase inhibitor (NNRTi), Protease inhibitor (Pi), Cell Entry inhibitor (Ci), and a combination thereof.

49. The method of claim 46, wherein the inhibitor is a Nucleoside analog Reverse Transcriptase inhibitor (NRTi) selected from the group consisting of AZT, 3TC, FTC, ABC, ddC, ZDV, TDF, ddI, DFC, DAPD, alovidine, elvucitabine, and a combination thereof.

50. The method of claim 46, wherein the inhibitor is a Non-Nucleoside analog Reverse Transcriptase inhibitor (NNRTi).

51. The method of claim 46, wherein the inhibitor is a Non-Nucleoside analog Reverse Transcriptase inhibitor (NNRTi) selected from the group consisting of DLV, EFV, NVP, calanolide A, etravirine, TMC-278, BMS-561390, capravirine, and a combination thereof.

52. The method of claim 46, wherein the inhibitor is a Protease inhibitor (Pi).

53. The method of claim 46, wherein the inhibitor is a Protease inhibitor (Pi) selected from the group consisting of APV, TPV, IDV, SQV, LPV, FPV, RTV, ATZ, NFV, brecanavir, darunavir, and a combination thereof.

54. The method of claim 46, wherein the inhibitor is a Cell Entry inhibitor (Ci).

55. The method of claim 46, wherein the inhibitor is a Cell Entry inhibitor (Ci) selected from the group consisting of Fuzeon, ENF, aplaviroc, maraviroc, vicriviroc, T-1249, PRO-542, TNX-355, SCH-C, and a combination thereof.

56. The method of claim 46, wherein the animal is a human.
